SEQUENCE LISTING (60 sequences)

SEQ ID NO: 1 - protein, 113 aa, synthetic construct
  REGION 1..113: note = Synthetic Construct
QSVKESGGRLVTPGTPLTLTCKVSGFSLSSYYMNWVRQAPGKGLEWIGIMFPNGKIYYATWAKGRFTISKTSTTVDLKIISPTTEDTATYFCTGDDSGDVNIWGPGTLVTVSS

SEQ ID NO: 2 - protein, 113 aa, synthetic construct
  REGION 1..113: note = Synthetic Construct
DIVLTQTASPVSAAVGGTVTINCQSSQSVYTNNRLAWYQQKPGQPAKEMIYGASTLPSGVSSRFKGSGSGTQFALTISDVQCDDAATYYCLGTYDCLSADCLAFGGGTKLEIK

SEQ ID NO: 3 - protein, 5 aa, synthetic construct
  REGION 1..5: note = Synthetic Construct
SYYMN

SEQ ID NO: 4 - protein, 14 aa, synthetic construct
  REGION 1..14: note = Synthetic Construct
IMFPNGKIYYATWA

SEQ ID NO: 5 - protein, 8 aa, synthetic construct
  REGION 1..8: note = Synthetic Construct
DDSGDVNI

SEQ ID NO: 6 - protein, 13 aa, synthetic construct
  REGION 1..13: note = Synthetic Construct
QSSQSVYTNNRLA

SEQ ID NO: 7 - protein, 7 aa, synthetic construct
  REGION 1..7: note = Synthetic Construct
GASTLPS

SEQ ID NO: 8 - protein, 13 aa, synthetic construct
  REGION 1..13: note = Synthetic Construct
LGTYDCLSADCLA

SEQ ID NO: 9 - protein, 112 aa, synthetic construct
  REGION 1..112: note = Synthetic Construct
QSVEESGGGLVTPGGALTLTCTASGFSLNRHWMNWVRQAPGKGLEWIGAINESGRTYYATWAKGRFFISKTTTTVDLKITSPTTADTATYFCVRDDDVSNFWGPGTLVTVSS

SEQ ID NO: 10 - protein, 113 aa, synthetic construct
  REGION 1..113: note = Synthetic Construct
DPMLTQTPSSVSAAVGGTVSINCQSTKSVYKYNHLSWYQQKPGQPPKQLIFPASTLQSGVPSRFSGSGSGTQFTLTISDVQCDDAATYYCLGLYDCRSGDCNVFGGGTKLEIK

SEQ ID NO: 11 - protein, 5 aa, synthetic construct
  REGION 1..5: note = Synthetic Construct
RHWMN

SEQ ID NO: 12 - protein, 14 aa, synthetic construct
  REGION 1..14: note = Synthetic Construct
AINESGRTYYATWA

SEQ ID NO: 13 - protein, 7 aa, synthetic construct
  REGION 1..7: note = Synthetic Construct
DDDVSNF

SEQ ID NO: 14 - protein, 13 aa, synthetic construct
  REGION 1..13: note = Synthetic Construct
QSTKSVYKYNHLS

SEQ ID NO: 15 - protein, 7 aa, synthetic construct
  REGION 1..7: note = Synthetic Construct
PASTLQS

SEQ ID NO: 16 - protein, 13 aa, synthetic construct
  REGION 1..13: note = Synthetic Construct
LGLYDCRSGDCNV

SEQ ID NO: 17 - protein, 114 aa, synthetic construct
  REGION 1..114: note = Synthetic Construct
QKQLMESGGRLVTPGTPLTLTCKVSGFSLSSYYMNWVRQAPGKGLEWIGIMFPNGKIYYATWAKGRFTISKTSTTVDLKIISPTTEDTATYFCTGDDSGDVNIWGPGTLVTVSS

SEQ ID NO: 18 - protein, 113 aa, synthetic construct
  REGION 1..113: note = Synthetic Construct
DIVLTQTASPVSAAVGGTVTINCQSSQSVYTNNRLAWYQQKPGQPAKEMIYGASTLPSGVSSRFKGSGSGTQFALTISDVQCDDAATYYCLGEFDCTSADCFVFGGGTEVVVK

SEQ ID NO: 19 - protein, 5 aa, synthetic construct
  REGION 1..5: note = Synthetic Construct
SYYMN

SEQ ID NO: 20 - protein, 14 aa, synthetic construct
  REGION 1..14: note = Synthetic Construct
IMFPNGKIYYATWA

SEQ ID NO: 21 - protein, 8 aa, synthetic construct
  REGION 1..8: note = Synthetic Construct
DDSGDVNI

SEQ ID NO: 22 - protein, 13 aa, synthetic construct
  REGION 1..13: note = Synthetic Construct
QSSQSVYTNNRLA

SEQ ID NO: 23 - protein, 7 aa, synthetic construct
  REGION 1..7: note = Synthetic Construct
GASTLPS

SEQ ID NO: 24 - protein, 13 aa, synthetic construct
  REGION 1..13: note = Synthetic Construct
LGEFDCTSADCFV

SEQ ID NO: 25 - protein, 113 aa, synthetic construct
  REGION 1..113: note = Synthetic Construct
QSVEESRGRLVTPGTPLTLTCKVSGFSLSSYYMNWVRQAPGKGLEWIGIMFPNGKIYYATWAKGRFTISKTSTTVDLKIISPTTEDTATYFCTGDDSGDVNIWGPGTLVTVSS

SEQ ID NO: 26 - protein, 113 aa, synthetic construct
  REGION 1..113: note = Synthetic Construct
DPMLTQTASPVSAAVGGTVTINCQSSQSVYSNNRLAWYQQKPGQPPKEMIYGASTLPSGVSSRFKGSGSGTQFTLTISDVQCDDAATYYCLGTYDCLSADCLAFGGGTEVVVK

SEQ ID NO: 27 - protein, 5 aa, synthetic construct
  REGION 1..5: note = Synthetic Construct
SYYMN

SEQ ID NO: 28 - protein, 14 aa, synthetic construct
  REGION 1..14: note = Synthetic Construct
IMFPNGKIYYATWA

SEQ ID NO: 29 - protein, 8 aa, synthetic construct
  REGION 1..8: note = Synthetic Construct
DDSGDVNI

SEQ ID NO: 30 - protein, 13 aa, synthetic construct
  REGION 1..13: note = Synthetic Construct
QSSQSVYSNNRLA

SEQ ID NO: 31 - protein, 7 aa, synthetic construct
  REGION 1..7: note = Synthetic Construct
GASTLPS

SEQ ID NO: 32 - protein, 13 aa, synthetic construct
  REGION 1..13: note = Synthetic Construct
LGTYDCLSADCLA

SEQ ID NO: 33 - protein, 114 aa, synthetic construct
  REGION 1..114: note = Synthetic Construct
  VARIANT 1..5: note = Xaa = Any Amino Acid
  VARIANT 8: note = Xaa = Any Amino Acid
  VARIANT 10: note = Xaa = Any Amino Acid
  VARIANT 16..17: note = Xaa = Any Amino Acid
  VARIANT 23..24: note = Xaa = Any Amino Acid
  VARIANT 30..33: note = Xaa = Any Amino Acid
  VARIANT 50..54: note = Xaa = Any Amino Acid
  VARIANT 56..57: note = Xaa = Any Amino Acid
  VARIANT 68: note = Xaa = Any Amino Acid
  VARIANT 73: note = Xaa = Any Amino Acid
  VARIANT 81: note = Xaa = Any Amino Acid
  VARIANT 86: note = Xaa = Any Amino Acid
  VARIANT 94..95: note = Xaa = Any Amino Acid
  VARIANT 98..101: note = Xaa = Any Amino Acid
  VARIANT 103: note = Xaa = Any Amino Acid
XXXXXESXGXLVTPGXXLTLTCXXSGFSLXXXXMNWVRQAPGKGLEWIGXXXXXGXXYYATWAKGRFXISKTXTTVDLKIXSPTTXDTATYFCXXDDXXXXNXWGPGTLVTVSS

SEQ ID NO: 34 - protein, 113 aa, synthetic construct
  REGION 1..113: note = Synthetic Construct
  VARIANT 2..3: note = Xaa = Any Amino Acid
  VARIANT 8: note = Xaa = Any Amino Acid
  VARIANT 10: note = Xaa = Any Amino Acid
  VARIANT 20: note = Xaa = Any Amino Acid
  VARIANT 26..27: note = Xaa = Any Amino Acid
  VARIANT 31..32: note = Xaa = Any Amino Acid
  VARIANT 34: note = Xaa = Any Amino Acid
  VARIANT 36: note = Xaa = Any Amino Acid
  VARIANT 46: note = Xaa = Any Amino Acid
  VARIANT 48..49: note = Xaa = Any Amino Acid
  VARIANT 51..52: note = Xaa = Any Amino Acid
  VARIANT 57: note = Xaa = Any Amino Acid
  VARIANT 61: note = Xaa = Any Amino Acid
  VARIANT 65: note = Xaa = Any Amino Acid
  VARIANT 74: note = Xaa = Any Amino Acid
  VARIANT 93..94: note = Xaa = Any Amino Acid
  VARIANT 97: note = Xaa = Any Amino Acid
  VARIANT 99: note = Xaa = Any Amino Acid
  VARIANT 102..103: note = Xaa = Any Amino Acid
  VARIANT 109..112: note = Xaa = Any Amino Acid
DXXLTQTXSXVSAAVGGTVXINCQSXXSVYXXNXLXWYQQKPGQPXKXXIXXASTLXSGVXSRFXGSGSGTQFXLTISDVQCDDAATYYCLGXXDCXSXDCXXFGGGTXXXXK

SEQ ID NO: 35 - skipped

SEQ ID NO: 36 - protein, 14 aa, synthetic construct
  REGION 1..14: note = Synthetic Construct
  VARIANT 1..5: note = Xaa = Any Amino Acid
  VARIANT 7..8: note = Xaa = Any Amino Acid
XXXXXGXXYYATWA

SEQ ID NO: 37 - skipped

SEQ ID NO: 38 - protein, 13 aa, synthetic construct
  REGION 1..13: note = Synthetic Construct
  VARIANT 3..4: note = Xaa = Any Amino Acid
  VARIANT 8..9: note = Xaa = Any Amino Acid
  VARIANT 11: note = Xaa = Any Amino Acid
  VARIANT 13: note = Xaa = Any Amino Acid
QSXXSVYXXNXLX

SEQ ID NO: 39 - protein, 7 aa, synthetic construct
  REGION 1..7: note = Synthetic Construct
  VARIANT 1: note = Xaa = Any Amino Acid
  VARIANT 6: note = Xaa = Any Amino Acid
XASTLXS

SEQ ID NO: 40 - protein, 13 aa, synthetic construct
  REGION 1..13: note = Synthetic Construct
  VARIANT 3: note = Xaa = Any Amino Acid
  VARIANT 4: note = Xaa = Any Amino Acid
  VARIANT 7: note = Xaa = Any Amino Acid
  VARIANT 9: note = Xaa = Any Amino Acid
  VARIANT 12..13: note = Xaa = Any Amino Acid
LGXXDCXSXDCXX

SEQ ID NO: 41 - protein, 14 aa, synthetic construct
  REGION 1..14: note = Synthetic Construct
LIFAGKGGQLEDGR

SEQ ID NO: 42 - protein, 20 aa, synthetic construct
  REGION 1..20: note = Synthetic Construct
TLSDYNIQKGGESTLHLVLR

SEQ ID NO: 43 - protein, 8 aa, synthetic construct
  REGION 1..8: note = Synthetic Construct
GGMLTNAR

SEQ ID NO: 44 - protein, 11 aa, synthetic construct
  REGION 1..11: note = Synthetic Construct
GGMALALAVTK

SEQ ID NO: 45 - protein, 21 aa, synthetic construct
  REGION 1..21: note = Synthetic Construct
GGLATFHGPGQLLCHPVLDLR

SEQ ID NO: 46 - protein, 9 aa, synthetic construct
  REGION 1..9: note = Synthetic Construct
GGMTSTYGR

SEQ ID NO: 47 - protein, 19 aa, synthetic construct
  REGION 1..19: note = Synthetic Construct
GGMFGSAPQRPVAMTTAQR

SEQ ID NO: 48 - protein, 17 aa, synthetic construct
  REGION 1..17: note = Synthetic Construct
GGMQLKPMEINPEMLNK

SEQ ID NO: 49 - protein, 25 aa, synthetic construct
  REGION 1..25: note = Synthetic Construct
GGMTGNAGEWCLMESDPGVFTELIK

SEQ ID NO: 50 - protein, 9 aa, synthetic construct
  REGION 1..9: note = Synthetic Construct
GGMQLKPME

SEQ ID NO: 51 - protein, 14 aa, synthetic construct
  REGION 1..14: note = Synthetic Construct
GGMTGNAGEWCLME

SEQ ID NO: 52 - protein, 436 aa, synthetic construct
  REGION 1..436: note = Synthetic Construct
QSVKESGGRLVTPGTPLTLTCKVSGFSLSSYYMNWVRQAPGKGLEWIGIMFPNGKIYYATWAKGRFTISKTSTTVDLKIISPTTEDTATYFCTGDDSGDVNIWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 53 - protein, 217 aa, synthetic construct
  REGION 1..217: note = Synthetic Construct
DIVLTQTASPVSAAVGGTVTINCQSSQSVYTNNRLAWYQQKPGQPAKEMIYGASTLPSGVSSRFKGSGSGTQFALTISDVQCDDAATYYCLGTYDCLSADCLAFGGGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 54 - protein, 435 aa, synthetic construct
  REGION 1..435: note = Synthetic Construct
QSVEESGGGLVTPGGALTLTCTASGFSLNRHWMNWVRQAPGKGLEWIGAINESGRTYYATWAKGRFFISKTTTTVDLKITSPTTADTATYFCVRDDDVSNFWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 55 - protein, 217 aa, synthetic construct
  REGION 1..217: note = Synthetic Construct
DPMLTQTPSSVSAAVGGTVSINCQSTKSVYKYNHLSWYQQKPGQPPKQLIFPASTLQSGVPSRFSGSGSGTQFTLTISDVQCDDAATYYCLGLYDCRSGDCNVFGGGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 56 - protein, 437 aa, synthetic construct
  REGION 1..437: note = Synthetic Construct
QKQLMESGGRLVTPGTPLTLTCKVSGFSLSSYYMNWVRQAPGKGLEWIGIMFPNGKIYYATWAKGRFTISKTSTTVDLKIISPTTEDTATYFCTGDDSGDVNIWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 57 - protein, 217 aa, synthetic construct
  REGION 1..217: note = Synthetic Construct
DIVLTQTASPVSAAVGGTVTINCQSSQSVYTNNRLAWYQQKPGQPAKEMIYGASTLPSGVSSRFKGSGSGTQFALTISDVQCDDAATYYCLGEFDCTSADCFVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 58 - protein, 436 aa, synthetic construct
  REGION 1..436: note = Synthetic Construct
QSVEESRGRLVTPGTPLTLTCKVSGFSLSSYYMNWVRQAPGKGLEWIGIMFPNGKIYYATWAKGRFTISKTSTTVDLKIISPTTEDTATYFCTGDDSGDVNIWGPGTLVTVSSASTKGPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 59 - protein, 217 aa, synthetic construct
  REGION 1..217: note = Synthetic Construct
DPMLTQTASPVSAAVGGTVTINCQSSQSVYSNNRLAWYQQKPGQPPKEMIYGASTLPSGVSSRFKGSGSGTQFTLTISDVQCDDAATYYCLGTYDCLSADCLAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 60 - skipped

(19) United States
(12) Patent Application Publication
(10) Pub. No.: US 2024/0109958 A1
(43) Pub. Date: Apr. 4, 2024

(54) ANTI-UBIQUITINATION ANTIBODIES AND METHODS OF USE

(71) Applicant: Genentech, Inc., South San Francisco, CA (US)

(72) Inventors: Donald Scott KIRKPATRICK, South San Francisco, CA (US); James Thomas KOERBER, San Mateo, CA (US); Christopher Williamson DAVIES, Oakland, CA (US)

(73) Assignee: Genentech, Inc., South San Francisco, CA (US)

(21) Appl. No.: 18/537,389

(22) Filed: Dec. 12, 2023

Related U.S. Application Data
(63) Continuation of application No. PCT/US2022/072995, filed on Jun. 16, 2022.
(60) Provisional application No. 63/212,075, filed on Jun. 17, 2021.

(51) Int. Cl. C07K 16/18; C07K 16/00; C40B 30/04; G01N 33/68

(52) U.S. Cl. CPC C07K 16/18; C07K 16/005; C40B 30/04; G01N 33/6845; C07K 2317/20; C07K 2317/34; C07K 2317/55; C07K 2317/56; C07K 2317/565; C07K 2317/622

(57) ABSTRACT

Provided herein are antibodies that bind to a peptide of an N-terminally ubiquitinated polypeptide and methods of screening for such antibodies. Also provided herein are detection and enrichment methods using such antibodies for detecting or enriching a peptide of an N-terminally ubiquitinated polypeptide.

### CROSS REFERENCE TO RELATED APPLICATIONS

This application is a continuation of National Phase application under 35 U.S.C. § 371 of International Application No. PCT/US2022/072995, filed internationally on Jun. 16, 2022, which claims priority benefits to U.S. Provisional Application No. 63/212,075 filed Jun. 17, 2021, the contents of each of which are incorporated herein by reference in their entirety.

### REFERENCE TO AN ELECTRONIC SEQUENCE LISTING

The content of the electronic sequence listing (146392052301seglist.xml; Size: 94,373 bytes; and Date of Creation: Dec. 5, 2023) is herein incorporated by reference in its entirety.

### FIELD OF THE INVENTION

The present invention relates to antibodies that bind to a peptide of an N-terminally ubiquitinated polypeptide, and methods of using the same.

### BACKGROUND OF THE INVENTION

Protein ubiquitination is a complex post-translational modification that regulates diverse cellular functions including protein homeostasis, DNA damage response, innate and adaptive immunity, the cell cycle, and inflammatory signaling (Komander, D. & Rape, M. Annul Rev Biochem 81, 203-229 (2012); Yau, R. & Rape, M. Nat Cell Biol 18, 579-586 (2016); Swatek, K. N. & Komander, D. Cell Res 26, 399-422 (2016); Dittmar, G. & Winklhofer, K. F. Front Chem 7, 915 (2020)). The covalent attachment of ubiquitin (Ub) to protein substrates occurs through the concerted activity of three enzymes: an E1 Ub-activating enzyme, an E2 Ub-conjugating enzyme, and an E3 Ub ligase (Deshaies, R. J. & Joazeiro, C. A. P. Annu Rev Biochem 78, 399-434 (2009); Schulman, B. A. & Harper, J. W. Nat Rev Mol Cell Bio 10, 319-331 (2009); Ye, Y. & Rape, M. Nat Rev Mol Cell Bio 10, 755-764 (2009)). Ub itself has seven lysine residues (K6, K11, K27, K29, K33, K48, and K63) and an N-terminus, all of which are amenable to conjugation (Komander, D. & Rape, M. Annu Rev Biochem 81, 203-229 (2012)). K48 and K63-linked polyubiquitin chains are the most well studied, with the traditional view being that K48-linked Ub chains mark proteins for proteasomal degradation, while K63-linked Ub chains have a protein scaffolding role (Swatek, K. N. & Komander, D. Cell Res 26, 399-422 (2016); Hershko, A. & Ciechanover, A. Annu Rev Biochem 67, 425-479 (1998); Chen, Z. J. & Sun, L. J. Mol Cell 33, 275-286 (2009)). Furthermore, studies have shown that mixed-linkage and branched Ub chains exist and can serve as more potent functional signals than homeotypic K48- or K-63-linked Ub chains (Kirkpatrick, D. S. et al. Nat Cell Biol 8, 700-710 (2006); Emmerich, C. H. et al. Proc National Acad Sci 110, 15247-15252 (2013); Meyer, H. J. & Rape, M. Cell 157, 910-921 (2014)). Conjugation of Ub to the ε-amino group of lysine residues is the most common form of ubiquitination. This type of conjugation forms a K-ε-GG motif in which the C-terminal glycine residues of the Ub peptide (“GG”) bind to the ε-amino group of lysine (“K-s”). Other acceptor residues such as Thr, Ser, Cys, and the α-amino group of substrate N-termini have been identified and are considered to be non-canonical ubiquitination targets (Cadwell, K. & Coscoy, L. Science 309, 127-130 (2005); Wang, X. et al. J Cell Biology 177, 613-624 (2007); Ciechanover, A. & Ben-Saadon, R. Trends Cell Biol 14, 103-106 (2004)). The biological significance of these non-canonical ubiquitinations is not well understood.

Upon its initial discovery, N-terminal Ub was posited to serve as a protein degradation signal (Breitschopf, K. et al., Embo J 17, 5964-5973 (1998); Bloom, J. et al., Cell 115, 71-82 (2003); Coulombe, P. et al., Mol Cell Biol 24, 6140-6150 (2004)). These studies showed that engineered proteins lacking lysine residues or naturally occurring lysine-less proteins were still subject to proteasomal degradation, indirectly implicating N-terminal Ub as the degradation signal. Subsequent work demonstrated that N-terminally ubiquitinated proteins do not accumulate significantly upon proteasome inhibition, suggesting that N-terminal ubiquitination might have additional roles beyond promoting proteasome-mediated degradation (Akimov, V. et al., Nat Struct Mol Biol 25, 631-640 (2018)), such as assisting in the folding of nascent polypeptides (Finley, D. et al., Nature 338, 394-401 (1989)). With the exception of linear polyubiquitin chains formed by LUBAC, UBE2W is the only E2 Ub-conjugating or E3 ligase enzyme reported to form a peptide bond between the C-terminal Gly-76 of Ub and the α-amino group of substrate protein N-termini (Scaglione, K. M. et al., J Biol Chem 288, 18784-18788 (2013); Kirisako, T. et al. Embo J 25, 4877-4887 (2006)). In coordination with a ubiquitin ligase, current data suggest that UBE2W strictly monoubiquitinates protein substrates at their N-termini. These priming modifications can be elaborated by other E2/E3 complexes into N-terminally linked polyubiquitin chains (Tatham, M. H. et al., Biochem J 453, 137-145 (2013)). Interestingly, UBE2W contains a partially disordered C-terminus that is critical for recognition of substrates that have intrinsically disordered N-termini (Vittal, V. et al., Nat Chem Biol 11, 83-89 (2015)). Despite a growing understanding of N-terminal ubiquitination and the structural and biochemical properties of UBE2W, only a small set of N-terminally ubiquitinated UBE2W substrates have been identified. Accordingly, new strategies to identify N-terminally ubiquitinated proteins are needed to further elucidate the physiological consequences of this modification.

In particular, strategies to globally profile N-terminally ubiquitinated proteins that are compatible with mass spectrometry would be especially beneficial. Mass spectrometry (MS) is a powerful analytical tool for identifying and elucidating substrate specific ubiquitination at the amino acid residue level (Peng, J. et al. Nat Biotechnol 21, 921-926 (2003); Kim, W. et al. Mol Cell 44, 325-340 (2011); Wagner, S. A. et al. Mol Cell Proteomics 10, M111.013284 (2011)). One approach has been the generation of tools that specifically enrich for peptides bearing Ub C-terminal remnants generated upon enzymatic cleavage. For example, the development of a monoclonal antibody recognizing the tryptic Ub remnant consisting of isopeptide-linked diglycine attached to the side chain of lysine (K-ε-GG) allowed for the global profiling of ubiquitination sites (Kim, W. et al. Mol Cell 44, 325-340 (2011); Xu, G., et al., Nat Biotechnol 28, 868-873 (2010); Bustos, D., et al., Mol Cell proteomics 11, 1529-1540 (2012)). More recently, a monoclonal antibody was generated to recognize the extended LysC-generated remnant of Ub and distinguish between substrates conjugated to Ub versus other Ub-like proteins such as NEDD8 and ISG15 (Akimov, V. et al., Nat Struct Mol Biol 25, 631-640 (2018)). Other affinity-based enrichment or genetic tagging systems have also been developed (Akimov, V. et al., Nat Struct Mol Biol 25, 631-640 (2018); Peng, J. et al. Nat Biotechnol 21, 921-926 (2003); Akimov, V. et al., J Proteome Res 17, 296-304 (2017); Kliza, K. et al., Nat Methods 14, 504-512 (2017); Danielsen, J. M. R. et al. Mol Cell Proteomics 10, M110.003590 (2011); Hjerpe, R. et al., Embo Rep 10, 1250-1258 (2009); Akimov, V. et al., Mol Biosyst 7, 3223-3233 (2011)). Notably, a few of these strategies detect not only canonical K-ε-GG peptides, but also peptides corresponding to N-terminal ubiquitination (Akimov, V. et al., Nat Struct Mol Biol 25, 631-640 (2018); Akimov, V. et al., J Proteome Res 17, 296-304 (2017)). Previous quantitative proteomics data suggest that the relative abundance of N-terminal Ub linkages is exceedingly low under basal conditions, given the frequency of lysines within a typical protein and the fact that ˜80-90% of proteins can be acetylated on their N-termini, precluding N-terminal ubiquitination (Akimov, V. et al., Nat Struct Mol Biol 25, 631-640 (2018); Arnesen, T. et al. Proc National Acad Sci 106, 8157-8162 (2009); Aksnes, H. et al., Cell Reports 10, 1362-1374 (2015)). Accordingly, there is a need in the art for antibodies capable of specifically detecting and enriching peptides unique to N-terminally ubiquitinated proteins.

### BRIEF SUMMARY

In one aspect, the present invention provides an antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG).

In some embodiments, the antibody binds to a peptide comprising an N-terminal sequence selected from the group consisting of GGA, GGE, GGF, GGG, GGH, GGI, GGL, GGM, GGN, GGQ, GGS, GGT, GGV, and GGW.

In some embodiments, the antibody binds to a peptide comprising the N-terminal sequence of GGA, a peptide comprising the N-terminal sequence of GGE, a peptide comprising the N-terminal sequence of GGF, a peptide comprising the N-terminal sequence of GGG, a peptide comprising the N-terminal sequence of GGH, a peptide comprising the N-terminal sequence of GGI, a peptide comprising the N-terminal sequence of GGL, a peptide comprising the N-terminal sequence of GGM, a peptide comprising the N-terminal sequence of GGN, a peptide comprising the N-terminal sequence of GGQ, a peptide comprising the N-terminal sequence of GGS, a peptide comprising the N-terminal sequence of GGT, a peptide comprising the N-terminal sequence of GGV, and a peptide comprising the N-terminal sequence of GGW.

In some embodiments, the antibody is a rabbit, rodent, or goat antibody.

In some embodiments, the antibody is a full-length antibody or a Fab fragment.

In some embodiments, the antibody is conjugated to a detectable label.

In some embodiments, the label is selected from the group consisting of biotin, digoxigenin, and fluorescein.

In some embodiments, the antibody is immobilized on a solid support.

In some embodiments, the antibody is immobilized on a bead.

In some embodiments, the antibody comprises a variable heavy chain (VH) comprising an Asn at position 35, Val at position 37, Thr at position 93, Asn at position 101, and Trp at position 103 on one side, and a variable light chain (VL) comprising an Ala at position 34, a Tyr at position 36, and a Tyr at position 49, numbering according to Kabat.

In some embodiments, the antibody comprises a variable heavy chain (VH) and a variable light chain (VL), wherein the antibody comprise a CDRH1 comprising the amino acid sequence XXXMN (SEQ ID NO: 35); a CDRH2 comprising the amino acid sequence XXXXXGXXYYATWA (SEQ ID NO:36); and a CDRH3 comprising the amino acid sequence DDXXXXNX (SEQ ID NO:37); wherein the antibody comprises a CDRL1 comprising the amino acid sequence QSXXSVYXXNXLX (SEQ ID NO:38); a CDRL2 comprising the amino acid sequence XASTLXS (SEQ ID NO: 39); and a CDRL3 comprising the amino acid sequence LGXXDCXSXDCXX (SEQ ID NO:40); wherein X is any amino acid.

In some embodiments, the VH comprises the amino acid set forth in SEQ ID NO: 33 and the VL comprises the amino acid sequence set forth in SEQ ID NO:34.

In some embodiments, the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 1 and a CDRL1, CDRL2, and CDRL3 of a VL comprising the amino acid sequence set forth in SEQ ID NO: 2.

In some embodiments, the antibody comprises the CDRH1 amino acid sequence set forth in SEQ ID NO: 3; the CDRH2 amino acid sequence set forth in SEQ ID NO: 4; the CDRH3 amino acid sequence set forth in SEQ ID NO:5; the CDRL1 amino acid sequence set forth in SEQ ID NO: 6; the CDRL2 amino acid sequence set forth in SEQ ID NO:7; and the CDRL3 amino acid sequence set forth in SEQ ID NO:8.

In some embodiments, the VH comprise the amino acid sequence set forth in SEQ ID NO: 1 and the VL comprises the amino acid sequence set forth in SEQ ID NO:2.

In some embodiments, the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 9 and a CDRL1, CDRL2, and CDRL3 of a VL comprising the amino acid sequences set forth in SEQ ID NO: 10.

In some embodiments, the antibody comprises the CDRH1 amino acid sequence set forth in SEQ ID NO: 11; the CDRH2 amino acid sequence set forth in SEQ ID NO: 12; the CDRH3 amino acid sequence set forth in SEQ ID NO:13; the CDRL1 amino acid sequence set forth in SEQ ID NO: 14; the CDRL2 amino acid sequence set forth in SEQ ID NO:15; and the CDRL3 amino acid sequence set forth in SEQ ID NO: 16.

In some embodiments, the VH comprises the amino acid sequence set forth in SEQ ID NO: 9 the VL comprises the amino acid sequence set forth in SEQ ID NO:10.

In some embodiments, the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 17 and a CDRL1, CDRL2, and CDRL3 of a VL comprising the amino acid sequence set forth in SEQ ID NO: 18.

In some embodiments, the antibody comprises the CDRH1 amino acid sequence set forth in SEQ ID NO: 19; the CDRH2 amino acid sequence set forth in SEQ ID NO: 20; the CDRH3 amino acid sequence set forth in SEQ ID NO:21; the CDRL1 amino acid sequence set forth in SEQ ID NO: 22; the CDRL2 amino acid sequence set forth in SEQ ID NO:23; and the CDRL3 amino acid sequence set forth in SEQ ID NO:24.

In some embodiments, the VH comprises the amino acid set forth in SEQ ID NO: 17 and the VL comprises the amino acid set forth in SEQ ID NO:18.

In some embodiments, the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 25 and a CDRL1, CDRL2, and CDRL3 of a VL comprising the amino acid sequences set forth in SEQ ID NO: 26.

In some embodiments, the antibody comprises the CDRH1 amino acid sequence set forth in SEQ ID NO: 27; the CDRH2 amino acid sequence set forth in SEQ ID NO: 28; the CDRH3 amino acid sequence set forth in SEQ ID NO:29; a CDRL1 amino acid sequence set forth in SEQ ID NO: 30; the CDRL2 amino acid sequence set forth in SEQ ID NO:31; and the CDRL3 amino acid sequence set forth in SEQ ID NO:32.

In some embodiments, the VH comprises the amino acid sequence set forth in SEQ ID NO: 25 and the VL comprises the amino acid sequence set forth in SEQ ID NO: 26.

In another aspect, nucleic acid encoding the antibody of any one of paragraphs [0006]-[0029] is provided.

In another aspect, a host cell comprising the nucleic acid of paragraph [0030] is provided.

In another aspect, the present invention provides a method of screening for an antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-s-GG), comprising
i) providing an antibody library;
ii) positively selecting antibodies that bind to a peptide comprising the amino acid sequence GGX at the N-terminus, wherein X is any amino acid; and
iii) negatively selecting antibodies that bind to a peptide comprising the amino acid sequence K-ε-GG,
   thereby producing an antibody that specifically binds to a peptide comprising the amino acid GGX at the N-terminus, and does not bind to the amino acid sequence K-ε-GG.

In some embodiments, in step ii) antibodies that bind to a peptide comprising the amino acid sequence GGM at the N-terminus are positively selected.

In some embodiments, negatively selecting antibodies that bind to a peptide comprising the amino acid sequence K-ε-GG is performed simultaneously with step ii).

In some embodiments, negatively selecting antibodies that bind to a peptide comprising the amino acid sequence K-ε-GG before or after step ii).

In some embodiments, the library is a phage library or a yeast library.

In some embodiments, the library is produced by immunizing a mammal with a peptide library comprising peptides comprising the amino acid sequence GGM at the N-terminus.

In some embodiments, the mammal is a rabbit or a mouse.

In some embodiments, steps ii)-iii) are repeated two or more times.

In another aspect, an antibody produced by the method of any one of paragraphs [0032]-[0039] is provided.

In another aspect, the present invention provides a method of enriching N-terminally ubiquitinated peptides in a sample comprising a mixture of peptides, comprising:
i) contacting the sample with an antibody that binds to a peptide of an N-terminally ubiquitinated protein; and
ii) selecting antibody-bound peptides from the sample, wherein the antibody binds to the amino acid sequence GGX at the N-terminus, wherein the antibody does not bind to an amino acid sequence comprising a branched diglycine (K-ε-GG).

In some embodiments, the sample is a cell lysate.

In some embodiments, the method further comprises deleting a deubiquitinase in a cell and lysing the cell to produce the cell lysate.

In some embodiments, the method further comprises overexpressing a ubiquitin ligase in a cell and lysing the cell to produce the cell lysate.

In some embodiments, the cell lysate is incubated with trypsin to generate the peptides.

In some embodiments, the cell lysate is incubated with a bacterial or viral protease to generate the peptides.

In some embodiments, the method further comprises treating the cell with a proteasome inhibitor or an inhibitor of de-ubiquitination prior to lysate generation and incubation with trypsin or prior to incubation with the bacterial or viral protease.

In some embodiments, the method further comprises detecting the selected antibody-bound peptides.

In some embodiments, the antibody-bound peptides are detected by mass spectrometry.

In some embodiments, the antibody-bound peptides are detected by protein sequencing.

In some embodiments, the antibody-bound peptides are detected using a secondary antibody that binds to the antibody that binds to a peptide of an N-terminally ubiquitinated protein.

In another aspect, a library of N-terminally ubiquitinated peptides produced by the method of any one of paragraphs [0041]-[0051] is provided.

In another aspect, the present invention provides a method of detecting an N-terminally ubiquitinated peptide in a sample comprising a mixture of peptides comprising
i) incubating the sample with an enzyme to generate peptides;
ii) contacting the peptides with an antibody that binds to a peptide of an N-terminally ubiquitinated protein, and
iii) detecting the N-terminally ubiquitinated peptide, wherein the antibody binds to the amino acid sequence GGX at the N-terminus, wherein the antibody does not bind to an amino acid sequence comprising a branched diglycine (K-ε-GG).

In some embodiments, the N-terminally ubiquitinated peptide is detected using a secondary antibody that binds to the antibody that binds to a peptide of an N-terminally ubiquitinated protein.

In some embodiments, the sample is a cell lysate.

In some embodiments, the method further comprises overexpressing a ubiquitin ligase in a cell and lysing the cell to produce the cell lysate.

In some embodiments, the method further comprises deleting a deubiquitinase in a cell and lysing the cell to produce the cell lysate.

In some embodiments, the cell lysate is incubated with a bacterial or viral protease to generate the peptides.

In some embodiments, the method further comprises treating the cell with a proteasome inhibitor or an inhibitor of de-ubiquitination prior to lysate generation and incubation with the bacterial or viral protease.

In another aspect, the present invention provides a kit for detecting N-terminally ubiquitinated peptides in a sample comprising an antibody that binds to a peptide of an N-terminally ubiquitinated polypeptide and instructions for use, wherein the antibody that binds to a peptide of an N-terminally ubiquitinated polypeptide, wherein the antibody binds to the amino acid sequence GGX at the N-terminus, wherein the antibody does not bind to an amino acid sequence comprising a branched diglycine (K-ε-GG).

In some embodiments, the antibody that binds to a peptide of an N-terminally ubiquitinated polypeptide is conjugated to a detectable label.

In some embodiments, the detectable label is selected from the group consisting of biotin, digoxigenin, and fluorescein.

In some embodiments, the antibody is immobilized on a solid support.

In some embodiments, the antibody is immobilized on a bead.

In some embodiments, the kit further comprises a protease.

### BRIEF DESCRIPTION OF THE FIGURES

FIG. 1A shows a schematic summary of the immunization and phage panning strategy used to generate anti-GGX monoclonal antibodies (mAbs). FIG. 1B shows the chemical structure of Gly-Gly-Met (GGM; top) and K-ε-GG (bottom) peptides. FIG. 1C provides data from an enzyme-linked immunosorbent assay (ELISA) measuring the ability of polyclonal antibodies (pAb) from each of the eight rabbits to bind the GGM and K-ε-GG peptides, with streptavidin as control. The identity of the rabbit is shown on the x-axis, with, from left to right for each rabbit, individual bars representing the level of binding to GGM peptide, K-ε-GG peptide, and streptavidin. The optical density at 650 nm is shown on the y-axis. FIG. 1D shows an amino acid sequence alignment of the light chain variable regions (top) and heavy chain variable regions (bottom) of monoclonal antibodies 1C7, 2H2, 2E9, and 2B12. The light chain variable region alignment includes, from top to bottom, 1C7 (SEQ ID NO: 2), 2H2 (SEQ ID NO: 26), 2E9 (SEQ ID NO: 18), and 2B12 (SEQ ID NO: 10). The heavy chain variable region alignment includes, from top to bottom, 1C7 (SEQ ID NO: 1), 2H2 (SEQ ID NO: 25), 2E9 (SEQ ID NO: 17), and 2B12 (SEQ ID NO: 9). Numbering of the amino acid positions according to Kabat, and the positions of the CDRs are shown above each alignment. FIG. 1E provides data from an ELISA measuring the ability of 1C7, 2B12, 2E9, 2H2 and an anti-K-ε-GG mAb to bind the GGM and K-ε-GG peptides, with neutravidin as control. The identity of the antibody is shown on the x-axis with, from left to right for each antibody, individual bars representing the level of binding to GGM peptide, K-ε-GG peptide, and neutravidin. The optical density at 650 nm is shown on the y-axis, n=3, and the error bars show the standard deviation. FIG. 1F provides data from an ELISA measuring the ability of 1C7, 2B12, 2E9, and 2H2 to bind GGX peptides. All twenty amino acids except for cysteine were substituted at position “X”, as indicated on the y-axis. The identity of the antibody is shown on the x-axis. The darker shading corresponds to better binding (i.e., a higher optical density at 650 nm, as shown in the scale at right), the blank row is a streptavidin control, and n=3.

FIG. 2A shows a surface representation of the 1C7 Fab bound to GGM peptide (shown as a stick diagram). The positions of the 1C7 CDRs are labeled. FIG. 2B shows a cartoon representation of the 1C7 Fab bound to GGM peptide (shown as a stick diagram) enveloped within the electron density mesh, contoured at 16. FIG. 2C shows a detailed view of the interaction between the diglycine and the 1C7 Fab, showing a hydrogen bond network and contacts with both the light chain and heavy chain. The GGM peptide is shown as a stick diagram surrounded by a space-filling diagram. The heavy chain residues are shown above the GGM peptide, and light chain residues are shown below the GGM peptide. Amino acid residues on the antibody and GGM peptide are labeled. FIG. 2D shows a detailed view of the methionine recognition pocket located at the light chain-heavy chain interface, containing a mixture of hydrophobic and hydrophilic residues. The heavy chain residues are shown at top, and light chain residues are shown at bottom. Amino acid residues are labeled. FIG. 2E shows a Gly-Gly-pro (GGP) peptide (shown as a stick diagram) modelled into the structure of the 1C7 Fab and highlights steric clashes that likely prevent binding to the antibody. FIG. 2F shows a model of a pocket in 2B12 which may bind a Trp sidechain, with the HC Thr93Val and LC Leu96Asn residues indicated.

FIG. 3A shows a schematic of a workflow for immunoaffinity enrichment and mass spectrometry (MS) analysis of GGX peptides (GGX-IAP-LC-MS/MS). FIG. 3B shows extracted ion chromatograms (+/−10 ppm) for K48 and K63 K-ε-GG polyubiquitin chain linkage peptides LIFAGK^{GG}QLEDGR (SEQ ID NO: 41; left) and TLSDYNIQK^{GG}ESTLHLVLR (SEQ ID NO: 42; right) in anti-K-ε-GG antibody, anti-GGX antibody 2B12, and anti-GGX antibody 1C7 immunoaffinity enrichment MS experiments. The x-axis shows the time in minutes, and the y-axis shows the abundance of peptide. FIG. 3C shows extracted ion chromatograms (+/−10 ppm) for K48 and K63 K-ε-GG polyubiquitin chain linkage peptides LIFAGK^{GG}QLEDGR (SEQ ID NO: 41; left) and TLSDYNIQK^{GG}ESTLHLVLR (SEQ ID NO: 42; right) in anti-K-ε-GG antibody, anti-GGX antibody 2E9, and anti-GGX antibody 2H2 immunoaffinity enrichment MS experiments. The x-axis shows the time in minutes, and the y-axis shows the abundance of peptide. FIG. 3D shows extracted ion chromatograms (+/−10 ppm) for internal GGX peptides GGMLTNAR (SEQ ID NO: 43; left) and GGMoxALALAVTK (SEQ ID NO: 44; right) in anti-K-ε-GG antibody, anti-GGX antibody 2B12, and anti-GGX antibody 1C7 immunoaffinity enrichment MS experiments. The x-axis shows the time in minutes, and the y-axis shows the abundance of peptide. FIG. 3E shows extracted ion chromatograms (+/−10 ppm) for internal GGX peptides GGLATFHGPGQLLCHPVLDLR (SEQ ID NO: 45; left) and GGMTSTYGR (SEQ ID NO: 46; right) in anti-K-ε-GG antibody, anti-GGX 2E9 antibody, and anti-GGX 2H2 antibody immunoaffinity enrichment MS experiments. The x-axis shows the time in minutes, and the y-axis shows the abundance of peptide. FIG. 3F shows the number of immunoaffinity enriched internal GGX peptides with various amino acid residues at position X. The x-axis shows the amino acid residue at position X, and the y-axis shows the number of peptides. FIG. 3G shows WebLogos representing the sequence diversity of internal GGX peptides enriched by the anti-GGX mAbs 1C7 (upper left), 2H2 (upper right), 2B12 (lower left), and 2E9 (lower right). FIG. 3H shows extracted ion chromatograms (+/−4 ppm) for N-terminal GGX peptide ^{GG}MFGSAPQRPVAMTTAQR (SEQ ID NO: 47) in anti-K-ε-GG antibody, anti-GGX antibody 2B12, and anti-GGX antibody 1C7 immunoaffinity enrichment MS experiments. The x-axis shows the time in minutes, and the y-axis shows the abundance of peptide. FIG. 3I shows MS/MS spectrum identification of triply charged 654.9938 m/z N-terminal GGX modified peptide ^{GG}MFGSAPQRPVAMTTAQR (SEQ ID NO: 47). Detected b- and y-ions are labeled.

FIG. 4A shows a western blot of stable doxycycline-inducible UBE2W HEK293 cells at 24 hours post doxycycline treatment. Below, a western blot of tubulin is shown as a control. FIG. 4B shows a volcano plot showing differential N-terminal protein ubiquitination data for UBE2W overexpression (UBE2Woe) versus control conditions in the label free GGX-MS experiment. The x-axis shows the log₂ fold change (FC), and the y-axis shows the −log₁₀ (P value). Each data point represents one protein, and a representative set of protein names shown. Protein-level cutoffs set at log₂ fold change (FC) >1.0 and −log₁₀ P value >1.3 (P<0.05) are marked by dashed lines. FIG. 4C shows a western blot of stable doxycycline-inducible UBE2W/RNF4 HEK293 cells at 24 hours post doxycycline treatment. FIG. 4D shows a scatter plot showing proteins with differential N-terminal protein ubiquitination in UBE2W overexpression versus control (UBE2Woe-ctrl) and combo versus RNF4 overexpression (combo-RNF4oe) contrasts in tandem mass tagging (TMT) 11-plex GGX-IAP-LC-MS/MS experiment. The x-axis shows the log₂ fold change (FC) of UBE2W overexpression versus control, and the y-axis shows the log₂ fold change (FC) of combo versus RNF4 overexpression. Sizes of the data points are scaled with P values. The dashed lines correspond to those where −log₁₀ P value >1.3 (P<0.05) in both contrasts. All experiments were performed with replicates (control n=3, UBE2W only n=3, RNF4 only n=2, and Combo n=3). FIG. 4E shows volcano plots showing differential N-terminal protein ubiquitination data for UBE2W overexpression versus control (UBE2Woe—Ctrl; left), combo versus control (Combo—Ctrl; center), and combo versus RNF4 overexpression (Combo—RNF4oe; right) conditions in label free GGX-MS experiment. In each plot, the x-axis shows the log₂ fold change (FC), and the y-axis shows the −log₁₀ (P value). Each data point represents one protein. Protein-level cutoffs set at log₂ fold change >1.0 and −log₁₀ P value >1.3 (P<0.05) marked by dashed lines. FIG. 4F shows a scatter plot showing proteins with differential N-terminal protein ubiquitination in UBE2W overexpression versus control and combo versus RNF4 overexpression contrasts in label free GGX-MS experiment. The x-axis shows the log₂ fold change (FC) of UBE2W overexpression versus control (UBE2Woe-ctrl), and the y-axis shows the log₂ fold change (FC) of combo versus RNF4 overexpression (Combo-RNF4oe). Sizes of the data points are scaled with P values. The dashed lines correspond to those where −log₁₀ P value >1.3 (P<0.05) in both contrasts. FIG. 4G shows an area proportional Venn diagram comparing the number of identified putative UBE2W substrates from each of the three MS experiments. The first label free quantitative (LFQ) experiment (LFQ_1) is the upper left circle, the second LFQ experiment (LFQ_2) is the lower left circle, and the TMT experiment is the circle on the right. The numbers represent the number of substrates that were identified within each experiment or shared between multiple experiments. The Venn diagram was generated using the BioVenn web application (Hulsen, T. et al., BMC Genomics 9, 488 (2008)). FIG. 4H shows histograms displaying relative N-terminal ubiquitination abundances, with individual bars showing values for individual TMT-11plex channels corresponding to biological replicates. Each bar represents an average of technical replicates (n=2). Results are shown for, from left to right, RS7, MIP18, and QKI. In each plot, the x-axis represents the sample from the experiment, and the y-axis shows the relative abundance. FIG. 4I shows western blots of wild type, doxycycline-inducible UBE2W/RNF4, and doxycycline-inducible UBE2W^{W144E}/RNF4 stable HEK293 cells transfected with constructs encoding for five lysine-less mutants of putative UBE2W substrates. An HA tag was fused at the C-terminal of each construct for protein detection using an anti-HA tag antibody. The arrows indicate the modified forms of each substrates. Results are representative of three independent experiments. Below each blot, a western blot of tubulin is shown as a control. FIG. 4J shows an analysis of the second position of immunoaffinity enriched UBE2W substrates after the initiator methionine. The x-axis shows the amino acid residue at position X after the initiator methionine, and the y-axis shows the number of peptides.

FIG. 5A shows extracted ion chromatograms (+/−10 ppm) for N-terminal tryptic GGX peptides ^{GG}MQLKPMEINPEMLNK (SEQ ID NO: 48) and ^{GG}MTGNAGEWCLMESDPGVFTELIK (SEQ ID NO: 49) of UCHL1 (left) and UCHL5 (right), respectively, in control (CTLR; upper plot) and UBE2W overexpression (UBE2W oe; lower plot) conditions from GGX-IAP-LC-MS/MS experiment. The x-axis shows the time in minutes, and the y-axis shows the abundance of peptide. FIG. 5B shows MS/MS spectra identifications of N-terminal tryptic GGX modified peptides ^{GG}MQLKPMEINPEMLNK (SEQ ID NO: 48) (triply charged, 643.9907 m/z; left) and ^{GG}MTGNAGEWCLMESDPGVFTELIK (SEQ ID NO: 49) (triply charged, 900.4094 m/z; right). Detected b- and y-ions are labeled. FIG. 5C shows extracted ion chromatograms (+/−10 ppm) for N-terminal semi-tryptic GGX peptides ^{GG}MQLKPME (SEQ ID NO: 50) and ^{GG}MTGNAGEWCLME (SEQ ID NO: 51) of UCHL1 (left) and UCHL5 (right), respectively, in control (CTLR; upper plot) and UBE2W overexpression (UBE2Woe; lower plot) conditions from GGX-IAP-LC-MS/MS experiment. The x-axis shows the time in minutes, and the y-axis shows the abundance of peptide. FIG. 5D shows MS/MS spectra identifications of N-terminal semi-tryptic GGX modified peptides ^{GG}MQLKPME (SEQ ID NO: 50) (doubly charged, 495.7406 m/z; left) and ^{GG}MTGNAGEWCLME (SEQ ID NO: 51) (doubly charged, 756.7994 m/z; right). Detected b- and y-ions are labeled. FIG. 5E shows results of in vitro ubiquitination assays performed on lysine-less catalytically inactive UCHL1 (top) and UCHL5 (bottom). Two-hour reactions were carried out in the absence of UBE2W (lane 1 and 2) and presence of UBE2W (lane 3-5). All reactions were incubated with E1, E3 (RNF4), ATP/MgCl₂, and with (lane 2) or without (lane 5) ubiquitin. Results are representative of 3 independent experiments. FIG. 5F shows western blots of doxycycline-inducible UBE2W/RNF4 and UBE2W^{W144E}/RNF4 HEK293 cells at 24 hours post doxycycline treatment. Endogenous UCHL1 expression was analyzed with an anti-UCHL1 antibody. Results are representative of 3 independent experiments. FIG. 5G shows western blots of doxycycline-inducible UBE2W/RNF4 HEK293 cells at 24 hours post doxycycline treatment. Cells were additionally treated with bortezomib (10 μM, 5 hours) before cell harvest. FIG. 5H shows western blots of doxycycline-inducible UBE2W/RNF4 HEK293 cells at 24 hours post doxycycline treatment. Cells were additionally treated with cycloheximide (10 μg/ml) for the indicated times before cell harvest.

FIG. 6A shows a schematic diagram of the Bio-Layer interferometry (BLI) experiments. UCHL1 and UCHL5 interactions were measured using immobilized biotin-ubiquitin (Ub) on a streptavidin (SA) biosensor, measuring the association of free UCHL1 or UCHL5 and Ub-UCHL1 or UCHL5 with the Ub surface. FIG. 6B shows combined steady state binding curves for (from highest to lowest response) UCHL1, Ub^{G76V}_UCHL1, Ub^{I44A,G76V}_UCHL1, UCHL5, Ub^{G76V}_UCHL5, and Ub^{I44A,G76V}_UCHL5. The x-axis shows the concentration of analyte in nM, and the y-axis shows Rmax values in nm for each concentration of analyte. Each assay was performed in triplicate. FIG. 6C shows representative sensorgrams showing the binding of Ub to wild-type UCHL1 (top), the N-terminally ubiquitinated mimetic (Ub^{G76V}_UCHL1; center), or Ub^{I44A,G76V}_UCHL1 (bottom).

FIG. 6D shows representative sensorgrams showing the binding of Ub to wild-type UCHL5 (top), the N-terminally ubiquitinated mimetic (Ub^{G76V}_UCHL5; center), or Ub^{I44A,G76V}-UCHL5 (bottom). FIG. 6E shows the results of activity assays performed with ubiquitin-Rho110 and UCHL1 constructs (left) and UCHL5 constructs (right). For UCHL1 and UCHL5, samples included the wild-type protein (shown as circles), catalytically dead mutant (C90S or C88S; shown as open squares), the N-terminally ubiquitinated mimetics (Ub^{G76V}; shown as closed squares), or Ub^{I144,AG76V} (triangles pointing up). In each plot, the x-axis shows the concentration of ubiquitin-Rho110 in μM, and the y-axis shows the reaction rate in μM s⁻¹. Data are reported as best-fit values with standard errors from nonlinear regression fit. Results are representative of two independent experiments. FIG. 6F shows the results of ubiquitin vinyl sulfone assays. UCHL1, its N-terminally ubiquitinated mimetic, Ub^{G67V}-UCHL1, and Ub^{I44A,G67V}_UCHL1 (left) and UCHL5, its N-terminally ubiquitinated mimetic, Ub^{G67V}_UCHL5, and Ub^{I44A,G67V}_UCHL5 (right) were allowed to react with the suicide probe Ubiquitin-Vinyl Sulfone (Ub-VS) for the indicated time points (0, 5, 15, or 30 minutes). The arrow indicates the band associated with the N-terminally ubiquitinated mimetic reacting with HA-Ub-VS. Results are representative of three independent experiments. FIG. 6G shows western blots of HEK293 cells transfected with, from left to right, empty vector, wild-type UCHL1, Ub^{G76V}_UCHL1, UCHL1^{C90S} (catalytically inactive mutant), Ub^{G76V}-UCHL1^{C90S}, or UCHL1^{D30K}(non-Ub binding mutant) at 24 hours post doxycycline treatment. Monoubiquitin is indicated by the arrow. Results are representative of two independent experiments. A western blot of tubulin is shown as a control.

FIG. 7A shows western blots of ubiquitin and UBE2W levels in samples with or without treatment with the proteasome inhibitor Bortezomib, with or without UBE2W overexpression. A western blot of tubulin is shown as a control. FIG. 7B shows volcano plots showing differential N-terminal protein ubiquitination data for UBE2W overexpression versus Ctrl (left), and combo versus Bortezomib treatment (Combo-Btz; left). In each plot, the x-axis shows the log 2 fold change (FC), and the y-axis shows the −log₁₀ (P value). Each data point represents one protein. FIG. 7C shows a heatmap showing the label free peak area for, from left to right along the x-axis, two replicates of control, two replicates of Bortezomib (Btz) treatment, two replicates of UBE2W overexpression, and two replicates of the RNF4/UBE2W combination. The y-axis shows the levels of the indicated proteins. FIG. 7D shows a sample correlation table showing the correlation between two replicates of control, two replicates of Bortezomib (Btz) treatment, two replicates of UBE2W overexpression, and two replicates of the RNF4/UBE2W combination.

### DETAILED DESCRIPTION

### I. Definitions

“GGX” as used herein refers to a peptide comprising the amino acid sequence (from N- to C-terminus) Gly-Gly-X at the N terminus, wherein X is any amino acid.

An “anti-GGX antibody” as used herein refers to an antibody that binds to a polypeptide comprising a GGX peptide at the N-terminus.

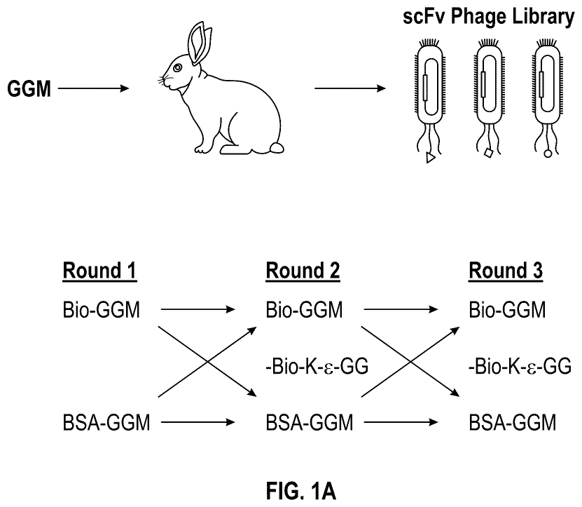

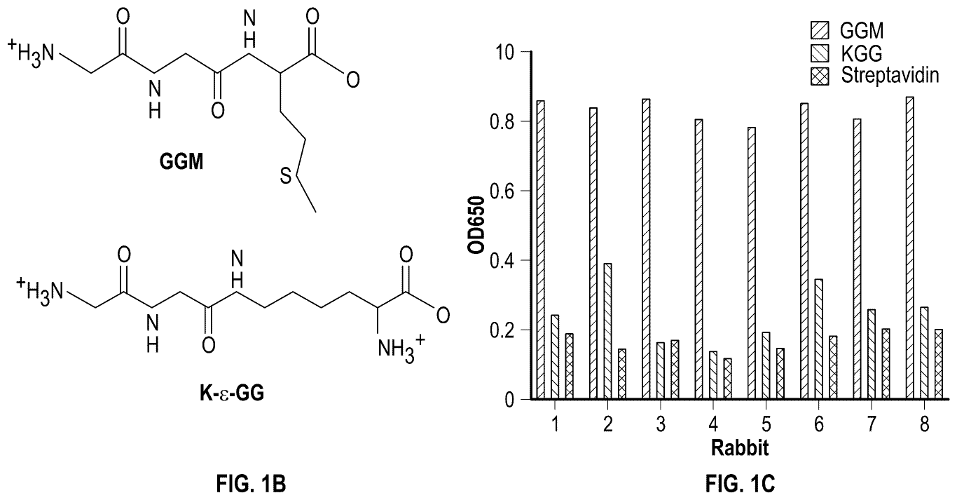

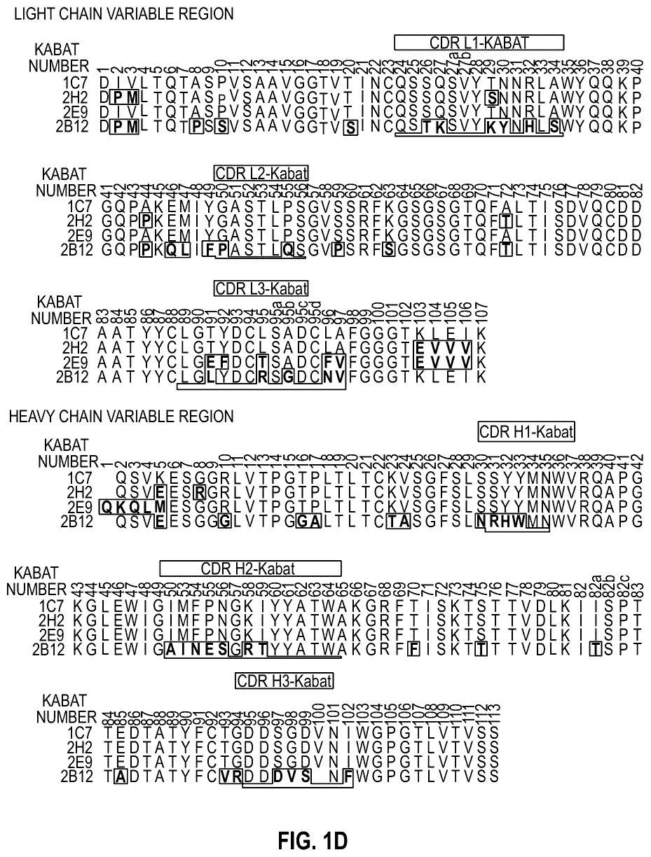

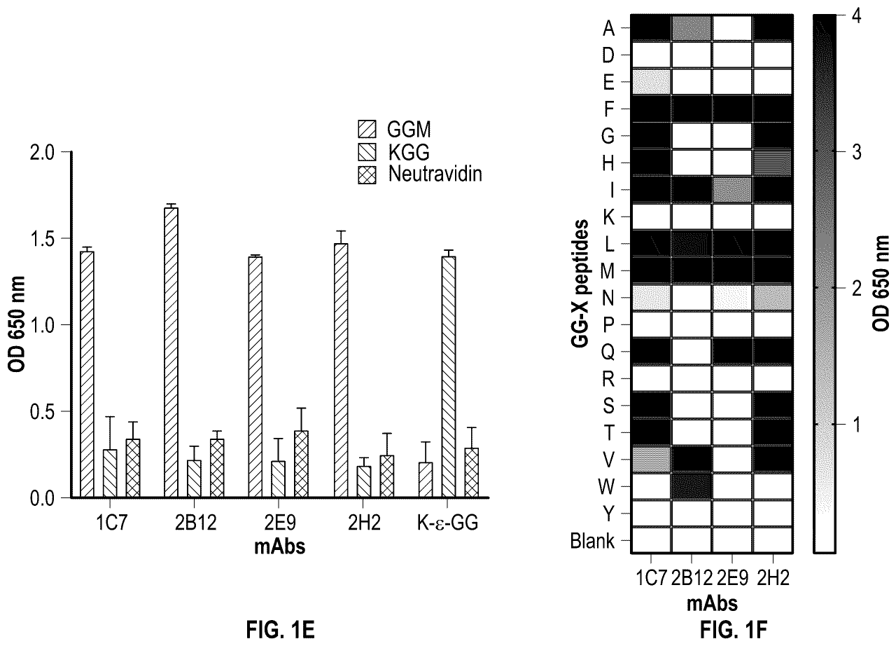

“K-ε-GG” and as used herein refers to two glycine residues (“GG”) bound to the ε-amino group of a lysine residue (“K-s”). K-ε-GG is a signature of the conjugation of ubiquitin to the ε-amino group of lysine residues, which is the most common form of ubiquitination. The three C-terminal residues of ubiquitin are Arg-Gly-Gly, and, in canonical ubiquitination, the C-terminal glycine residue is conjugated to a lysine residue in the target polypeptide. Upon digestion with trypsin, ubiquitin is cleaved after the arginine residue, resulting in a Gly-Gly dipeptide remnant on the conjugated lysine. Therefore, the presence of a K-ε-GG peptide (also termed a “K-ε-GG di-glycine remnant” or a “branched diglycine”) in a trypsin-digested polypeptide indicates the prior conjugation of ubiquitin to the ε-amino group of a lysine residue in the polypeptide. The chemical structure of K-ε-GG is provided in FIG. 1B.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “chimeric” antibody refers to an antibody in which a portion of the heavy and/or light chain is derived from a particular source or species, while the remainder of the heavy and/or light chain is derived from a different source or species.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementarity-determining regions (CDRs). (See, e.g., Kindt et al. Kuby Immunology, 6^{th} ed., W. H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Exemplary CDRs (CDRL1, CDRL2, CDRL3, CDRH1, CDRH2, and CDRH3) occur at amino acid residues 24-34 of L1, 50-56 of L2, 89-97 of L3, 31-35B of H1, 50-65 of H2, and 95-102 of H3. (Kabat et al., Sequences of proteins of Immunological Interest, 5th Ed. Public Health Service, National Institutes of Health, Bethesda, MD (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDRL1, a-CDRL2, a-CDRL3, a-CDRH1, a-CDRH2, and a-CDRH3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, CDR residues and other residues in the variable domain (e.g., FR residues) are numbered herein according to Kabat et al., supra.

The “Fab” fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of proteins of Immunological Interest, 5th Ed. Public Health Service, National Institutes of Health, Bethesda, MD, 1991.

“Framework” or “FR” refers to variable domain residues other than CDR residues. The FR of a variable domain generally consists of four FR domains: FR1, FR2, FR3, and FR4. Accordingly, the CDR and FR sequences generally appear in the following sequence in VH (or VL): FR1-CDRH1(L1)-FR2-CDRH2(L2)-FR3-CDRH3(L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “immunoconjugate” is an antibody conjugated to one or more heterologous molecule(s), including but not limited to a cytotoxic agent.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the singular form “a” “an”, and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

### II. Compositions and Methods

In one aspect, the present disclosure provides antibodies that interact with or otherwise bind to a region, such as an epitope, of an N-terminally ubiquitinated polypeptide.

A. Antibodies that Binds to a Peptide of an N-Terminally Ubiquitinated Polypeptide

1. N-Terminally Ubiquitinated Polypeptides

The present disclosure is based in part on the development of antibodies capable of specifically detecting and enriching N-terminally ubiquitinated polypeptides. As described in Example 1, the inventors anticipated that a substantial portion of potential N-terminally ubiquitinated polypeptides would be nascent polypeptides with non-acetylated, intact initiator methionines that, upon trypsin digestion, would yield peptides with a diglycine modification prior to the start methionine residue. Accordingly, a selection was designed to identify antibodies capable of selectively enriching for tryptic peptides containing a diglycine sequence at their N-termini (see FIG. 1A and Example 1). As described in detail herein, a rabbit immune phage strategy was used to generate novel antibodies that selectively recognize peptides bearing an N-terminal diglycine-motif, but not the branched diglycine remnant generated by trypsin digestion of ubiquitin-conjugated lysines (K-ε-GG; see FIG. 1A and Example 1). Using a combination of biochemical and structural methods, it is shown herein that these antibodies predominately recognize the N-terminal diglycine with a relaxed selectivity for the third amino acid, enabling these mAbs to bind to a broad range of peptide sequences (see Example 2).

Two enzymes capable of generating N-terminally ubiquitinated polypeptides are known in the art. First, the ubiquitin-conjugating enzyme UBE2W has been reported to have an N-terminal ubiquitin (Scaglione, K. M. et al., J Biol Chem 288, 18784-18788 (2013)). Second, a ubiquitin ligase, the linear ubiquitin chain assembly complex (“LUBAC”) has been reported to comprise an N-terminal ubiquitin chain (Kirisako, T. et al., Embo J 25, 4877-4887 (2006)). Accordingly, in some embodiments the N-terminally ubiquitinated polypeptide is UBE2W. In some embodiments the N-terminally ubiquitinated polypeptide is LUBAC.

In one aspect, the antibodies of the present disclosure were used to identify N-terminally ubiquitinated polypeptides (see Examples 3-4). In some embodiments, an antibody of the present disclosure selectively enriches an N-terminally ubiquitinated polypeptide from a cell lysate (e.g., a HEK293 cell lysate, or lysate of a HEK293 cell with inducible UBE2W expression). In some embodiments, the N-terminally ubiquitinated polypeptide comprises a diglycine at the initiator methionine or at the neo-N-terminus. In some embodiments, the polypeptide comprises the amino acid sequence GGX at the N-terminus of the polypeptide. In some embodiments, enrichment is calculated as described in Example 3 or Example 4. In some embodiments, the antibody enriches the polypeptide from the cell lysate to a level that is greater than 1 log₂(fold change) (e.g., greater than 1, 1.5, 2, 3, 4, 5, 6, 7, 8, 9, or 10 log₂(fold change)). In some embodiments, the antibody enriches the polypeptide from the cell lysate to a statistical significance that is p<0.05 (e.g., p<0.05, p<0.04, p<0.03, p<0.02, p<0.01, p<0.005, p<0.001, p<0.0001, or p<0.00001). In some embodiments, enrichment is calculated relative to the abundance of the polypeptide in cell lysate that has not been contacted with an antibody that binds to a peptide of an N-terminally ubiquitinated polypeptide. In some embodiments in which the polypeptide is selectively enriched from the lysate of a HEK293 cell with inducible UBE2W expression, the polypeptide is enriched upon induction of UBE2W expression. In some embodiments, the N-terminally ubiquitinated polypeptide is any one of the polypeptides listed in Table 7 or Table 8. In some embodiments, the N-terminally ubiquitinated polypeptide is selected from the group consisting of human DCTP1, human F13A, human HNRPK, human PUR9, human RFA1, human RPB7, human SI1IP, and human UCHL5. In some embodiments, the N-terminally ubiquitinated polypeptide is selected from the group consisting of human AAAT, human AES, human AIG1, human ARF1, human ARL5B, human BABA2, human BUB3, human C1TC, human C2AIL, human C9J470, human CD81, human CDC45, human DCTP1, human DHRSX, human DMKN, human E2AK1, human EF1B, human F13A, human FA60A, human FBRL, human FLOT1, human GCYB1, human GOT1B, human GPAA1, human HIKES, human HNRPK, human IMPA3, human LAT3, human LAT4, human LRWD1, human MED25, human MFS12, human MIP18, human MMGT1, human MOONR, human NARR, human NDUB6, human NENF, human NOL6, human NOP10, human NUDC, human P121A, human PIGC, human PLBL2, human PRDX1, human PRDX2, human PUR9, human QKI, human RAD21, human RCAS1, human REEP1, human RFA1, human RPB1, human RPB7, human RS29, human RS7, human S11IP, human SGMR1, human T179B, human TAF1, human TCPG, human TF3C4, human TM127, human TMM97, human TMX2, human TSN13, human TSN3, human TTC27, human UBAC1, human UBAC2, human UCHL1, human UCHL5, human VKOR1, human VRK3, human ZDH12, human ZN253, and human ZN672. In some embodiments, the N-terminally ubiquitinated polypeptide is human UCHL1. In some embodiments, the N-terminally ubiquitinated polypeptide is human UCHL5. In some embodiments, the N-terminally ubiquitinated polypeptide is selected from the group consisting of Uniprot Accession Nos. Q15758, Q08117, Q9NVV5, P84077, Q96KC2, Q9NXR7, 043684, P11586, Q96HQ2, C9J470, P60033, 075419, Q9H773, Q8N5I4, Q6EOU4-8, Q9BQI3, P24534, P00488, Q9NP50, P22087, 075955, Q02153, Q9Y3E0, 043292, Q53FT3, P61978, Q9NX62, 075387, Q8N370, Q9UFC0, Q71SY5, Q6NUT3, Q9Y3D0, Q8N4V1, Q2KHM9, P0DI83, 095139, Q9UMX5, Q9H6R4, Q9NPE3, Q9Y266, Q96HA1, Q92535, Q8NHP8, Q06830, P32119, P31939, Q96PU8, 060216, 000559, Q9H902, P27694, P24928, P62487, P62273, P62081, Q8N1F8, Q99720, Q7Z7N9, P21675, P49368, Q9UKN8, 075204, Q5BJF2, Q9Y320, 095857, 060637, Q6P3X3, Q9BSL1, Q8NBM4, P09936, Q9Y5K5, Q9BQB6, Q8IV63, Q96GR4, 075346, and Q499Z4. In some embodiments, the N-terminally ubiquitinated polypeptide comprises a disordered N-terminus.

2. Antibodies that Binds to a Peptide of an N-Terminally Ubiquitinated Polypeptide

Antibodies that bind to a peptide of an N-terminally ubiquitinated polypeptide are provided herein. In some embodiments, the antibody binds to the amino acid sequence GGX at the N-terminus of the peptide, wherein the antibody does not bind to an amino acid sequence comprising a branched diglycine (K-ε-GG). In some embodiments, X is any amino acid.

Provided herein are antibodies that bind to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG). In some embodiments, the antibody binds to the amino acid sequence GGX at the N-terminus of the peptide as determined by an enzyme-linked immunosorbent assay (ELISA). In some embodiments, the antibody binds to the amino acid sequence GGX at the N-terminus of the peptide to a greater extent than it binds an amino acid sequence comprising a branched diglycine (K-ε-GG), as determined by an ELISA. In some embodiments, the antibody binds to the amino acid sequence GGX at the N-terminus of the peptide at a level of binding that is greater than 2, 3, 4, 5, 6, 7, 8, 9, or 10-fold the level of binding of the antibody to an amino acid sequence comprising a branched diglycine (K-ε-GG), including any value or range between these values. In some embodiments, the antibody binds to the amino acid sequence GGX at the N-terminus of the peptide at a level of binding that is greater than 2, 3, 4, 5, 6, 7, 8, 9, or 10-fold the level of binding of the antibody to a control sample (e.g., neutravidin or streptavidin), including any value or range between these values. In some embodiments, the amino acid sequence GGX at the N-terminus of the peptide is GGM. An exemplary method of measuring binding is provided in Example 1 (see “Monoclonal antibody ELISAs”) and FIG. 1E.

In some embodiments, the antibody that binds to a peptide of an N-terminally ubiquitinated polypeptide binds to the amino acid sequence GGX at the N-terminus of the peptide with a greater affinity than a control antibody binds to the peptide. In some embodiments, the control antibody is an isotype control. In some embodiments, the control antibody is an anti-K-ε-GG antibody (e.g., the Cell Signaling Technology® PTMScan® Ubiquitin Remnant Motif antibody). In some embodiments, the antibody specifically binds to a peptide of an N-terminally ubiquitinated polypeptide in a western blot. In some embodiments, the antibody is capable of immunoprecipitating a peptide comprising an amino acid sequence GGX at the N-terminus of the peptide. In some embodiments, the antibody is capable of being co-crystallized with a peptide comprising an amino acid sequence GGX at the N-terminus of the peptide. In some embodiments, the antibody specifically binds to a peptide of an N-terminally ubiquitinated polypeptide in a surface plasmon resonance (SPR) assay.

In some embodiments, the antibody binds to a peptide of an N-terminally ubiquitinated polypeptide with a dissociation constant (K_{d}) that is less than 100, 10, 1, or 0.1 μM. In some embodiments, the antibody binds to a peptide of an N-terminally ubiquitinated polypeptide with a K_{d} that is about 0.1, 0.2, 0.3, 0.4, 0.5, 0.6, 0.7, 0.8, 0.9, 1, 2, 3, 4, 5, 6, 7, 8, 9, 10, 15, 20, 25, 50, 75, or 100 μM, including any value or range between these values. In some embodiments, the antibody binds to a peptide of an N-terminally ubiquitinated polypeptide with a K_{d} that is less than 100, 10, or 1 nM. In some embodiments, the antibody binds to a peptide of an N-terminally ubiquitinated polypeptide with a K_{d} that is about 5, 10, 20, 30, 40, 50, 60, 70, 80, 90, 100, 250, 500, 750, or 1000 nM, including any value or range between these values. In some embodiments, the K_{d} is measured using surface plasmon resonance (SPR). In some embodiments, the K_{d} is measured by measuring binding to a GGM peptide.

Provided herein are antibodies that do not bind to an amino acid sequence comprising a branched diglycine (K-ε-GG). In some embodiments, the antibody does not bind to an amino acid sequence comprising a branched diglycine (K-ε-GG), wherein binding of the antibody is not detectable over background or is at the same level as a negative control (e.g., the level of non-specific binding, or the level of binding neutravidin). In some embodiments, binding of the antibody to an amino acid sequence comprising a branched diglycine (K-ε-GG) is not detectable (e.g., not detectable by an ELISA, SPR assay, western blot, and/or immunoprecipitation).

In some embodiments, the antibody binds to an amino acid sequence comprising a branched diglycine (K-ε-GG) at a level that is less than 50%, 40%, 30%, 20%, 10% the level of binding of the antibody to an N-terminally ubiquitinated polypeptide, wherein the antibody binds to the amino acid sequence GGX at the N-terminus of the peptide, including any value or range between these values. In some embodiments, the antibody binds to an amino acid sequence comprising a branched diglycine (K-ε-GG) to the same extent that the antibody binds neutravidin. In some embodiments, the antibody binds to an amino acid sequence comprising a branched diglycine (K-ε-GG) to the same extent that the antibody binds streptavidin. In some embodiments, the level of binding of the antibody to an amino acid sequence comprising a branched diglycine (K-ε-GG) is no more than 1.1, 1.2, 1.3, 1.4, or 1.5-fold greater than the level of binding to a negative control sample (e.g., the level of binding to neutravidin or streptavidin). In some embodiments, the level of binding of the antibody to an amino acid sequence comprising a branched diglycine (K-ε-GG) is not statistically significantly different from the level of binding to a negative control sample (e.g., the level of binding to neutravidin or streptavidin).

In some embodiments, the antibody that binds to a peptide of an N-terminally ubiquitinated polypeptide binds to a peptide comprising an N-terminal sequence selected from the group consisting of GGA, GGE, GGF, GGG, GGH, GGI, GGL, GGM, GGN, GGQ, GGS, GGT, GGV, and GGW. In some embodiments, the antibody binds to a peptide comprising an N-terminal GGA sequence. In some embodiments, the antibody binds to a peptide comprising an N-terminal GGE sequence. In some embodiments, the antibody binds to a peptide comprising an N-terminal GGF sequence. In some embodiments, the antibody binds to a peptide comprising an N-terminal GGG sequence. In some embodiments, the antibody binds to a peptide comprising an N-terminal GGH sequence. In some embodiments, the antibody binds to a peptide comprising an N-terminal GGI sequence. In some embodiments, the antibody binds to a peptide comprising an N-terminal GGL sequence. In some embodiments, the antibody binds to a peptide comprising an N-terminal GGM sequence. In some embodiments, the antibody binds to a peptide comprising an N-terminal GGN sequence. In some embodiments, the antibody binds to a peptide comprising an N-terminal GGQ sequence. In some embodiments, the antibody binds to a peptide comprising an N-terminal GGS sequence. In some embodiments, the antibody binds to a peptide comprising an N-terminal GGT sequence. In some embodiments, the antibody binds to a peptide comprising an N-terminal GGV sequence. In some embodiments, the antibody binds to a peptide comprising an N-terminal GGW sequence. In some embodiments, the antibody binds to peptides comprising N-terminal sequences of GGA, GGE, GGF, GGG, GGH, GGI, GGL, GGM, GGN, GGQ, GGS, GGT, GGV, and GGW. In some embodiments, the antibody binds to a peptide comprising the N-terminal sequence of GGA, a peptide comprising the N-terminal sequence of GGE, a peptide comprising the N-terminal sequence of GGF, a peptide comprising the N-terminal sequence of GGG, a peptide comprising the N-terminal sequence of GGH, a peptide comprising the N-terminal sequence of GGI, a peptide comprising the N-terminal sequence of GGL, a peptide comprising the N-terminal sequence of GGM, a peptide comprising the N-terminal sequence of GGN, a peptide comprising the N-terminal sequence of GGQ, a peptide comprising the N-terminal sequence of GGS, a peptide comprising the N-terminal sequence of GGT, a peptide comprising the N-terminal sequence of GGV, and a peptide comprising the N-terminal sequence of GGW.

In some embodiments, the antibody binds to one or more peptides comprising N-terminal sequences selected from the group consisting of GGA, GGE, GGF, GGG, GGH, GGI, GGL, GGM, GGN, GGQ, GGS, GGT, GGV, and GGW, including any combination of the peptides. In some embodiments, the antibody binds to a peptide comprising an N-terminal GGA sequence, a peptide comprising an N-terminal GGE sequence, a peptide comprising an N-terminal GGF sequence, a peptide comprising an N-terminal GGG sequence, a peptide comprising an N-terminal GGH sequence, a peptide comprising an N-terminal GGI sequence, a peptide comprising an N-terminal GGL sequence, a peptide comprising an N-terminal GGM sequence, a peptide comprising an N-terminal GGN sequence, a peptide comprising an N-terminal GGQ sequence, a peptide comprising an N-terminal GGS sequence, a peptide comprising an N-terminal GGT sequence, and a peptide comprising an N-terminal GGV sequence. In some embodiments, the antibody binds to a peptide comprising an N-terminal GGA sequence, a peptide comprising an N-terminal GGF sequence, a peptide comprising an N-terminal GGI sequence, a peptide comprising an N-terminal GGL sequence, a peptide comprising an N-terminal GGM sequence, a peptide comprising an N-terminal GGV sequence, and a peptide comprising an N-terminal GGW sequence. In some embodiments, the antibody binds to a peptide comprising an N-terminal GGA sequence, a peptide comprising an N-terminal GGF sequence, a peptide comprising an N-terminal GGI sequence, a peptide comprising an N-terminal GGL sequence, a peptide comprising an N-terminal GGM sequence, a peptide comprising an N-terminal GGN sequence, and a peptide comprising an N-terminal GGQ sequence, a peptide comprising an N-terminal GGS sequence, and a peptide comprising an N-terminal GGT sequence. In some embodiments, the antibody binds to a peptide comprising an N-terminal GGA sequence, a peptide comprising an N-terminal GGE sequence, a peptide comprising an N-terminal GGF sequence, a peptide comprising an N-terminal GGG sequence, a peptide comprising an N-terminal GGH sequence, a peptide comprising an N-terminal GGI sequence, a peptide comprising an N-terminal GGL sequence, a peptide comprising an N-terminal GGM sequence, a peptide comprising an N-terminal GGN sequence, a peptide comprising an N-terminal GGQ sequence, a peptide comprising an N-terminal GGS sequence, a peptide comprising an N-terminal GGT sequence, and a peptide comprising an N-terminal GGV sequence. In some embodiments, the antibody binds to a peptide comprising an N-terminal GGA sequence, a peptide comprising an N-terminal GGE sequence, a peptide comprising an N-terminal GGF sequence, a peptide comprising an N-terminal GGG sequence, a peptide comprising an N-terminal GGH sequence, a peptide comprising an N-terminal GGI sequence, a peptide comprising an N-terminal GGL sequence, a peptide comprising an N-terminal GGM sequence, a peptide comprising an N-terminal GGN sequence, a peptide comprising an N-terminal GGQ sequence, a peptide comprising an N-terminal GGS sequence, a peptide comprising an N-terminal GGT sequence, a peptide comprising an N-terminal GGV sequence, and a peptide comprising an N-terminal GGW sequence. The specificities of exemplary antibodies are provided in FIG. 1F.

In some embodiments, the antibody is a rabbit antibody, a rodent antibody, or a goat antibody. In some embodiments, the antibody is a rabbit antibody that possesses an amino acid sequence which corresponds to that of an antibody produced by a rabbit or a rabbit cell or derived from a non-rabbit source that utilizes rabbit antibody repertoires or other rabbit antibody-encoding sequences. In some embodiments, the antibody is derived from a rabbit. In some embodiments, the antibody is derived from a New Zealand White Rabbit. In some embodiments, the antibody is derived from a rodent. In some embodiments, the antibody is derived from a goat. In some embodiments, the antibody comprises an Fc region derived from a rabbit, goat, or rodent antibody. In some embodiments, the antibody comprises an antibody fragment from a rabbit, goat, or rodent antibody.

In a further aspect of the invention, the antibody that binds to a peptide of an N-terminally ubiquitinated polypeptide according to any of the above embodiments is a monoclonal antibody, including a chimeric, humanized or human antibody. In some embodiments, the antibody that binds to a peptide of an N-terminally ubiquitinated polypeptide is an antibody fragment, e.g., a Fv, Fab, Fab′, scFv, diabody, or F(ab′)₂ fragment. In another embodiment, the antibody that binds to a peptide of an N-terminally ubiquitinated polypeptide is a full-length antibody, e.g., an intact IgG1 antibody or other antibody class or isotype as defined herein. In some embodiments, the antibody is a full-length antibody, a Fab fragment, or an scFv. In some embodiments, the antibody is of the IgA, IgD, IgE, IgG, or IgM class. In some embodiments, the antibody is of the IgG class. In some embodiments, the antibody is of the IgG class and has an IgG₁, IgG₂, IgG₃, or IgG₄ isotype. In some embodiments, the antibody is of the IgA class and has an IgA₁ or IgA₂ isotype.

In a further aspect of the invention, an antibody that binds to a peptide of an N-terminally ubiquitinated polypeptide according to any of the above embodiments or described herein is conjugated to a heterologous moiety, agent, or label. Examples of suitable labels are those numerous labels known for use in immunoassay, including moieties that may be detected directly, such as fluorochrome, chemiluminscent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare-earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, HRP, alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin (detectable by, e.g., avidin, streptavidin, streptavidin-HRP, and streptavidin-β-galactosidase with MUG), spin labels, bacteriophage labels, stable free radicals, and the like. In some embodiments, the label is selected from the group consisting of biotin, digoxigenin, and fluorescein. In some embodiments, the antibody that binds to a peptide of an N-terminally ubiquitinated polypeptide according to any of the above embodiments is conjugated to biotin.

In some embodiments, the antibody is immobilized on a solid support. In some embodiments, the antibody is immobilized on a bead. In some embodiments, immobilization is accomplished by insolubilizing the antibody by adsorption to a water-insoluble matrix or surface (U.S. Pat. No. 3,720,760) or non-covalent or covalent coupling (for example, using glutaraldehyde or carbodiimide cross-linking, with or without prior activation of the support with, e.g., nitric acid and a reducing agent as described in U.S. Pat. No. 3,645,852 or in Rotmans et al.; J. Immunol. Methods, 57:87-98 (1983)), or afterward, e.g., by immunoprecipitation. The solid support used for immobilization may be any inert support or carrier that is essentially water insoluble, including supports in the form of, e.g., surfaces, particles, porous matrices, etc. Examples of commonly used solid supports include small sheets, SEPHADEX® gels, polyvinyl chloride, plastic beads, and assay plates or test tubes manufactured from polyethylene, polypropylene, polystyrene, and the like, including 96-well microtiter plates, as well as particulate materials such as filter paper, agarose, cross-linked dextran, and other polysaccharides.

In some embodiments, the antibody that binds to a peptide of an N-terminally ubiquitinated polypeptide comprises a variable heavy chain (VH) comprising an Asn at position 35, Val at position 37, Thr at position 93, Asn at position 101, and Trp at position 103 on one side. In some embodiments, the antibody that binds to a peptide of an N-terminally ubiquitinated polypeptide comprises a variable light chain (VL) comprising an Ala at position 34, a Tyr at position 36, and a Tyr at position 49, numbering according to Kabat.

In some embodiments, the antibody that binds to a peptide of an N-terminally ubiquitinated polypeptide comprises a variable heavy chain (VH) and a variable light chain (VL), wherein the antibody comprise a CDRH1 comprising the amino acid sequence XXXMN (SEQ ID NO: 35); a CDRH2 comprising the amino acid sequence XXXXXGXXYYATWA (SEQ ID NO:36); and a CDRH3 comprising the amino acid sequence DDXXXXNX (SEQ ID NO:37); wherein the antibody comprises a CDRL1 comprising the amino acid sequence QSXXSVYXXNXLX (SEQ ID NO:38); a CDRL2 comprising the amino acid sequence XASTLXS (SEQ ID NO: 39); and a CDRL3 comprising the amino acid sequence LGXXDCXSXDCXX (SEQ ID NO:40); wherein X is any amino acid. In some embodiments, the VH comprises the amino acid set forth in SEQ ID NO: 33. In some embodiments, the VL comprises the amino acid sequence set forth in SEQ ID NO:34. In some embodiments, the VH comprises the amino acid set forth in SEQ ID NO: 33 and the VL comprises the amino acid sequence set forth in SEQ ID NO:34.

In some embodiments, the antibody that binds to a peptide of an N-terminally ubiquitinated polypeptide comprises one, two, three, four, five, or six CDRs of antibody 1C7 as shown in Table 2A and Table 2B. In some embodiments, the antibody comprises the VH and/or the VL of antibody 1C7 as shown in Table 3. In some embodiments, the antibody comprises the heavy chain and/or the light chain of antibody 1C7 as shown in Table 4.

In some embodiments, the antibody that binds a peptide of an N-terminally ubiquitinated polypeptide comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:1. In certain embodiments, a VH sequence contains substitutions (e.g., conservative substitutions), insertions, or deletions relative to the amino acid sequence of SEQ ID NO:1, but retains the ability to bind an N-terminally ubiquitinated polypeptide as the antibody comprising SEQ ID NO:1. In certain embodiments, a total of 1 to 13 amino acids have been substituted, inserted and/or deleted in SEQ ID NO: 1. In certain embodiments, substitutions, insertions, or deletions occur in regions outside the CDRs (i.e., in the FRs). In a particular embodiment, the VH comprises one, two or three CDRs selected from the group consisting of: (a) a CDRH1 comprising the amino acid sequence of SEQ ID NO:3, (b) a CDRH2 comprising the amino acid sequence of SEQ ID NO:4, and (c) a CDRH3 comprising the amino acid sequence of SEQ ID NO:5.

In another aspect, an antibody that binds a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:2. In certain embodiments, a VL sequence contains substitutions (e.g., conservative substitutions), insertions, or deletions relative to the amino acid sequence of SEQ ID NO:2, but retains the ability to bind an N-terminally ubiquitinated polypeptide as the antibody comprising SEQ ID NO:2. In certain embodiments, a total of 1 to 11 amino acids have been substituted, inserted and/or deleted in SEQ ID NO:2. In certain embodiments, the substitutions, insertions, or deletions occur in regions outside the CDRs (i.e., in the FRs). In a particular embodiment, the VL comprises one, two or three CDRs selected from the group consisting of (a) a CDRL1 comprising the amino acid sequence of SEQ ID NO:6; (b) a CDRL2 comprising the amino acid sequence of SEQ ID NO:7; and (c) a CDRL3 comprising the amino acid sequence of SEQ ID NO:8.

In one embodiment, the antibody that binds to a peptide of an N-terminally ubiquitinated polypeptide comprises a VL comprising the amino acid sequence of SEQ ID NO:2 and a VH comprising the amino acid sequence of SEQ ID NO:1.

In another aspect, an antibody that binds to a peptide of an N-terminally ubiquitinated polypeptide is provided, wherein the antibody comprises a VH comprising a CDRH1 comprising the amino acid sequence of SEQ ID NO:3, a CDRH2 comprising the amino acid sequence of SEQ ID NO:4, and a CDRH3 comprising the amino acid sequence of SEQ ID NO:5; and a VL comprising a CDRL1 comprising the amino acid sequence of SEQ ID NO:6, a CDRL2 comprising the amino acid sequence of SEQ ID NO:7, and a CDRL3 comprising the amino acid sequence of SEQ ID NO:8.

In another aspect, an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:1; and a VL CDR1, a VL CDR2, and a VL CDR3, respectively comprising the amino acid sequences of a CDR1, a CDR2, and a CDR3 of a VL having the sequence set forth in SEQ ID NO:2.

In some embodiments, the antibody comprises a heavy chain having at least 85%, 90%, 91%, 92%, 93%, 94%, 95%, 96%, 97%, 98%, 99%, or 100% sequence identity to the amino acid sequence of SEQ ID NO:52. In certain embodiments, the heavy chain sequence contains substitutions (e.g., conservative substitutions), insertions, or deletions relative to the amino acid sequence of SEQ ID NO:52, but retains the ability to bind an N-terminally ubiquitinated polypeptide as the antibody comprising SEQ ID NO:52. In certain embodiments, a total of 1 to 20 amino acids have been substituted, inserted and/or deleted in SEQ ID NO:52. In some embodiments, the antibody comprises a heavy chain comprising the amino acid sequence set forth in SEQ ID NO: 52.

In some embodiments, the antibody comprises a light chain having at least 85%, 90%, 91%, 92%, 93%, 94%, 95%, 96%, 97%, 98%, 99%, or 100% sequence identity to the amino acid sequence of SEQ ID NO:53. In certain embodiments, the light chain sequence contains substitutions (e.g., conservative substitutions), insertions, or deletions relative to the amino acid sequence of SEQ ID NO:53, but retains the ability to bind an N-terminally ubiquitinated polypeptide as the antibody comprising SEQ ID NO:53. In certain embodiments, a total of 1 to 20 amino acids have been substituted, inserted and/or deleted in SEQ ID NO:53. In some embodiments, the antibody comprises a light chain comprising the amino acid sequence set forth in SEQ ID NO: 53.

In some embodiments, the antibody that binds to a peptide of an N-terminally ubiquitinated polypeptide comprises one, two, three, four, five, or six CDRs of antibody 2B12 as shown in Table 2A and Table 2B. In some embodiments, the antibody comprises the VH and/or the VL of antibody 2B12 as shown in Table 3. In some embodiments, the antibody comprises the heavy chain and/or the light chain of antibody 2B12 as shown in Table 4.

In some embodiments, the antibody that binds a peptide of an N-terminally ubiquitinated polypeptide comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:9. In certain embodiments, a VH sequence contains substitutions (e.g., conservative substitutions), insertions, or deletions relative to the amino acid sequence of SEQ ID NO:9, but retains the ability to bind a peptide of an N-terminally ubiquitinated polypeptide as the antibody comprising SEQ ID NO:9. In certain embodiments, a total of 1 to 13 amino acids have been substituted, inserted and/or deleted in SEQ ID NO:9. In certain embodiments, substitutions, insertions, or deletions occur in regions outside the CDRs (i.e., in the FRs). In a particular embodiment, the VH comprises one, two or three CDRs selected from the group consisting of: (a) a CDRH1 comprising the amino acid sequence of SEQ ID NO:11, (b) a CDRH2 comprising the amino acid sequence of SEQ ID NO:12, and (c) a CDRH3 comprising the amino acid sequence of SEQ ID NO:13.

In another aspect, an antibody that binds a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 10. In certain embodiments, a VL sequence contains substitutions (e.g., conservative substitutions), insertions, or deletions relative to the amino acid sequence of SEQ ID NO:10, but retains the ability to bind a peptide of an N-terminally ubiquitinated polypeptide as the antibody comprising SEQ ID NO:10. In certain embodiments, a total of 1 to 11 amino acids have been substituted, inserted and/or deleted in SEQ ID NO:10. In certain embodiments, the substitutions, insertions, or deletions occur in regions outside the CDRs (i.e., in the FRs). In a particular embodiment, the VL comprises one, two or three CDRs selected from the group consisting of (a) a CDRL1 comprising the amino acid sequence of SEQ ID NO:14; (b) a CDRL2 comprising the amino acid sequence of SEQ ID NO:15; and (c) a CDRL3 comprising the amino acid sequence of SEQ ID NO:16.

In one embodiment, the antibody that binds to a peptide of an N-terminally ubiquitinated polypeptide comprises a VL comprising the amino acid sequence of SEQ ID NO:10 and a VH comprising the amino acid sequence of SEQ ID NO:9.

In another aspect, an antibody that binds to a peptide of an N-terminally ubiquitinated polypeptide is provided, wherein the antibody comprises a VH comprising a CDRH1 comprising the amino acid sequence of SEQ ID NO:11, a CDRH2 comprising the amino acid sequence of SEQ ID NO:12, and a CDRH3 comprising the amino acid sequence of SEQ ID NO:13; and a VL comprising a CDRL1 comprising the amino acid sequence of SEQ ID NO:14, a CDRL2 comprising the amino acid sequence of SEQ ID NO:15, and a CDRL3 comprising the amino acid sequence of SEQ ID NO:16.

In another aspect, an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:9; and a VL CDR1, a VL CDR2, and a VL CDR3, respectively comprising the amino acid sequences of a CDR1, a CDR2, and a CDR3 of a VL having the sequence set forth in SEQ ID NO:10.

In some embodiments, the antibody comprises a heavy chain having at least 85%, 90%, 91%, 92%, 93%, 94%, 95%, 96%, 97%, 98%, 99%, or 100% sequence identity to the amino acid sequence of SEQ ID NO:54. In certain embodiments, the heavy chain sequence contains substitutions (e.g., conservative substitutions), insertions, or deletions relative to the amino acid sequence of SEQ ID NO:54, but retains the ability to bind an N-terminally ubiquitinated polypeptide as the antibody comprising SEQ ID NO:54. In certain embodiments, a total of 1 to 20 amino acids have been substituted, inserted and/or deleted in SEQ ID NO:52. In some embodiments, the antibody comprises a heavy chain comprising the amino acid sequence set forth in SEQ ID NO: 54.

In some embodiments, the antibody comprises a light chain having at least 85%, 90%, 91%, 92%, 93%, 94%, 95%, 96%, 97%, 98%, 99%, or 100% sequence identity to the amino acid sequence of SEQ ID NO:55. In certain embodiments, the light chain sequence contains substitutions (e.g., conservative substitutions), insertions, or deletions relative to the amino acid sequence of SEQ ID NO:55, but retains the ability to bind an N-terminally ubiquitinated polypeptide as the antibody comprising SEQ ID NO:55. In certain embodiments, a total of 1 to 20 amino acids have been substituted, inserted and/or deleted in SEQ ID NO:55. In some embodiments, the antibody comprises a light chain comprising the amino acid sequence set forth in SEQ ID NO: 55.

In some embodiments, the antibody that binds to a peptide of an N-terminally ubiquitinated polypeptide comprises one, two, three, four, five, or six CDRs of antibody 2E9 as shown in Table 2A and Table 2B. In some embodiments, the antibody comprises the VH and/or the VL of antibody 2E9 as shown in Table 3. In some embodiments, the antibody comprises the heavy chain and/or the light chain of antibody 2E9 as shown in Table 4.

In some embodiments, the antibody that binds a peptide of an N-terminally ubiquitinated polypeptide comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:17. In certain embodiments, a VH sequence contains substitutions (e.g., conservative substitutions), insertions, or deletions relative to the amino acid sequence of SEQ ID NO:17, but retains the ability to bind a peptide of an N-terminally ubiquitinated polypeptide as the antibody comprising SEQ ID NO:17. In certain embodiments, a total of 1 to 13 amino acids have been substituted, inserted and/or deleted in SEQ ID NO:17. In certain embodiments, substitutions, insertions, or deletions occur in regions outside the CDRs (i.e., in the FRs). In a particular embodiment, the VH comprises one, two or three CDRs selected from the group consisting of: (a) a CDRH1 comprising the amino acid sequence of SEQ ID NO:19, (b) a CDRH2 comprising the amino acid sequence of SEQ ID NO:20, and (c) a CDRH3 comprising the amino acid sequence of SEQ ID NO:21.

In another aspect, an antibody that binds a peptide of a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 18. In certain embodiments, a VL sequence contains substitutions (e.g., conservative substitutions), insertions, or deletions relative to the amino acid sequence of SEQ ID NO:18, but retains the ability to bind an N-terminally ubiquitinated polypeptide as the antibody comprising SEQ ID NO:18. In certain embodiments, a total of 1 to 11 amino acids have been substituted, inserted and/or deleted in SEQ ID NO:18. In certain embodiments, the substitutions, insertions, or deletions occur in regions outside the CDRs (i.e., in the FRs). In a particular embodiment, the VL comprises one, two or three CDRs selected from the group consisting of (a) a CDRL1 comprising the amino acid sequence of SEQ ID NO:22; (b) a CDRL2 comprising the amino acid sequence of SEQ ID NO:23; and (c) a CDRL3 comprising the amino acid sequence of SEQ ID NO:24.

In one embodiment, the antibody that binds to a peptide of an N-terminally ubiquitinated polypeptide comprises a VL comprising the amino acid sequence of SEQ ID NO:18 and a VH comprising the amino acid sequence of SEQ ID NO:17.

In another aspect, an antibody that binds to a peptide of an N-terminally ubiquitinated polypeptide is provided, wherein the antibody comprises a VH comprising a CDRH1 comprising the amino acid sequence of SEQ ID NO:19, a CDRH2 comprising the amino acid sequence of SEQ ID NO:20, and a CDRH3 comprising the amino acid sequence of SEQ ID NO:21; and a VL comprising a CDRL1 comprising the amino acid sequence of SEQ ID NO:22, a CDRL2 comprising the amino acid sequence of SEQ ID NO:23, and a CDRL3 comprising the amino acid sequence of SEQ ID NO:24.

In another aspect, an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:17; and a VL CDR1, a VL CDR2, and a VL CDR3, respectively comprising the amino acid sequences of a CDR1, a CDR2, and a CDR3 of a VL having the sequence set forth in SEQ ID NO:18.

In some embodiments, the antibody comprises a heavy chain having at least 85%, 90%, 91%, 92%, 93%, 94%, 95%, 96%, 97%, 98%, 99%, or 100% sequence identity to the amino acid sequence of SEQ ID NO:56. In certain embodiments, the heavy chain sequence contains substitutions (e.g., conservative substitutions), insertions, or deletions relative to the amino acid sequence of SEQ ID NO:56, but retains the ability to bind an N-terminally ubiquitinated polypeptide as the antibody comprising SEQ ID NO:56. In certain embodiments, a total of 1 to 20 amino acids have been substituted, inserted and/or deleted in SEQ ID NO:56. In some embodiments, the antibody comprises a heavy chain comprising the amino acid sequence set forth in SEQ ID NO: 56.

In some embodiments, the antibody comprises a light chain having at least 85%, 90%, 91%, 92%, 93%, 94%, 95%, 96%, 97%, 98%, 99%, or 100% sequence identity to the amino acid sequence of SEQ ID NO:57. In certain embodiments, the light chain sequence contains substitutions (e.g., conservative substitutions), insertions, or deletions relative to the amino acid sequence of SEQ ID NO:57, but retains the ability to bind an N-terminally ubiquitinated polypeptide as the antibody comprising SEQ ID NO:57. In certain embodiments, a total of 1 to 20 amino acids have been substituted, inserted and/or deleted in SEQ ID NO:53. In some embodiments, the antibody comprises a light chain comprising the amino acid sequence set forth in SEQ ID NO: 57.

In some embodiments, the antibody that binds to a peptide of an N-terminally ubiquitinated polypeptide comprises one, two, three, four, five, or six CDRs of antibody 2H2 as shown in Table 2A and Table 2B. In some embodiments, the antibody comprises the VH and/or the VL of antibody 2H2 as shown in Table 3. In some embodiments, the antibody comprises the heavy chain and/or the light chain of antibody 2H2 as shown in Table 4.

In some embodiments, the antibody that binds a peptide of a peptide of an N-terminally ubiquitinated polypeptide comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:25. In certain embodiments, a VH sequence contains substitutions (e.g., conservative substitutions), insertions, or deletions relative to the amino acid sequence of SEQ ID NO:25, but retains the ability to bind a peptide of an N-terminally ubiquitinated polypeptide as the antibody comprising SEQ ID NO:25. In certain embodiments, a total of 1 to 13 amino acids have been substituted, inserted and/or deleted in SEQ ID NO:25. In certain embodiments, substitutions, insertions, or deletions occur in regions outside the CDRs (i.e., in the FRs). In a particular embodiment, the VH comprises one, two or three CDRs selected from the group consisting of: (a) a CDRH1 comprising the amino acid sequence of SEQ ID NO:27, (b) a CDRH2 comprising the amino acid sequence of SEQ ID NO:28, and (c) a CDRH3 comprising the amino acid sequence of SEQ ID NO:29.

In another aspect, an antibody that binds a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:26. In certain embodiments, a VL sequence contains substitutions (e.g., conservative substitutions), insertions, or deletions relative to the amino acid sequence of SEQ ID NO:26, but retains the ability to bind a peptide of an N-terminally ubiquitinated polypeptide as the antibody comprising SEQ ID NO:26. In certain embodiments, a total of 1 to 11 amino acids have been substituted, inserted and/or deleted in SEQ ID NO:26. In certain embodiments, the substitutions, insertions, or deletions occur in regions outside the CDRs (i.e., in the FRs). In a particular embodiment, the VL comprises one, two or three CDRs selected from the group consisting of (a) a CDRL1 comprising the amino acid sequence of SEQ ID NO:30; (b) a CDRL2 comprising the amino acid sequence of SEQ ID NO:31; and (c) a CDRL3 comprising the amino acid sequence of SEQ ID NO:32.

In one embodiment, the antibody that binds to a peptide of an N-terminally ubiquitinated polypeptide comprises a VL comprising the amino acid sequence of SEQ ID NO:26 and a VH comprising the amino acid sequence of SEQ ID NO:25.

In another aspect, an antibody that binds to a peptide of an N-terminally ubiquitinated polypeptide is provided, wherein the antibody comprises a VH comprising a CDRH1 comprising the amino acid sequence of SEQ ID NO:27, a CDRH2 comprising the amino acid sequence of SEQ ID NO:28, and a CDRH3 comprising the amino acid sequence of SEQ ID NO:29; and a VL comprising a CDRL1 comprising the amino acid sequence of SEQ ID NO:30, a CDRL2 comprising the amino acid sequence of SEQ ID NO:31, and a CDRL3 comprising the amino acid sequence of SEQ ID NO:32.

In another aspect, an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:25; and a VL CDR1, a VL CDR2, and a VL CDR3, respectively comprising the amino acid sequences of a CDR1, a CDR2, and a CDR3 of a VL having the sequence set forth in SEQ ID NO:26.

In some embodiments, the antibody comprises a heavy chain having at least 85%, 90%, 91%, 92%, 93%, 94%, 95%, 96%, 97%, 98%, 99%, or 100% sequence identity to the amino acid sequence of SEQ ID NO:58. In certain embodiments, the heavy chain sequence contains substitutions (e.g., conservative substitutions), insertions, or deletions relative to the amino acid sequence of SEQ ID NO:58, but retains the ability to bind an N-terminally ubiquitinated polypeptide as the antibody comprising SEQ ID NO:58. In certain embodiments, a total of 1 to 20 amino acids have been substituted, inserted and/or deleted in SEQ ID NO:58. In some embodiments, the antibody comprises a heavy chain comprising the amino acid sequence set forth in SEQ ID NO: 58.

In some embodiments, the antibody comprises a light chain having at least 85%, 90%, 91%, 92%, 93%, 94%, 95%, 96%, 97%, 98%, 99%, or 100% sequence identity to the amino acid sequence of SEQ ID NO:59. In certain embodiments, the light chain sequence contains substitutions (e.g., conservative substitutions), insertions, or deletions relative to the amino acid sequence of SEQ ID NO:59, but retains the ability to bind an N-terminally ubiquitinated polypeptide as the antibody comprising SEQ ID NO:59. In certain embodiments, a total of 1 to 20 amino acids have been substituted, inserted and/or deleted in SEQ ID NO:59. In some embodiments, the antibody comprises a light chain comprising the amino acid sequence set forth in SEQ ID NO: 59.

In another aspect, an antibody that binds to a peptide of an N-terminally ubiquitinated polypeptide is provided, wherein the antibody comprises a VH as in any of the embodiments provided above, and a VL as in any of the embodiments provided above.

In another aspect, provided herein is a composition comprising one or more of the antibodies that bind to a peptide of an N-terminally ubiquitinated polypeptide according to any of the above embodiments or described herein. In some embodiments, the composition comprising one more of the antibodies comprises a pharmaceutically acceptable carrier.

Also provided herein are methods of producing the antibodies that bind to a peptide of an N-terminally ubiquitinated polypeptide as described herein.

3. Antibody variants

In certain embodiments, amino acid sequence variants of the antibodies that bind to a peptide of an N-terminally ubiquitinated polypeptide provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., binding to a peptide of an N-terminally ubiquitinated polypeptide.

In certain embodiments, antibody variants having one or more amino acid substitutions are provided. Sites of interest for substitutional mutagenesis include the CDRs and FRs. Preferred, conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, pro;
aromatic: Trp, Tyr, Phe.

Non-conservative substitutions will entail exchanging a member of one of these classes for another class.

One type of substitutional variant involves substituting one or more hypervariable region residues or CDR residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Afol. Biol. 207: 179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDRH3 and CDRL3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions.

Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

B. Nucleic Acids, Vectors, Host Cells

Also provided herein is a nucleic acid encoding an antibody that binds to a peptide of an N-terminally ubiquitinated polypeptide. In some embodiments, the nucleic acid encodes any of the antibodies described herein.

In some embodiments, the nucleic acid encodes an antibody that binds to a peptide of an N-terminally ubiquitinated polypeptide comprises a variable heavy chain (VH) and a variable light chain (VL), wherein the antibody comprises a CDRH1 comprising the amino acid sequence XXXMN (SEQ ID NO: 35); a CDRH2 comprising the amino acid sequence XXXXXGXXYYATWA (SEQ ID NO:36); and a CDRH3 comprising the amino acid sequence DDXXXXNX (SEQ ID NO:37); wherein the antibody comprises a CDRL1 sequence set forth in SEQ ID NO: QSXXSVYXXNXLX (SEQ ID NO:38); a CDRL2 comprising the amino acid sequence XASTLXS (SEQ ID NO: 39); and a CDRL3 comprising the amino acid sequence LGXXDCXSXDCXX (SEQ ID NO:40); wherein X is any amino acid. In some embodiments, the nucleic acid encodes an antibody comprising a VH comprising the amino acid set forth in SEQ ID NO: 33. In some embodiments, the nucleic acid encodes an antibody comprising a VL comprising the amino acid sequence set forth in SEQ ID NO:34. In some embodiments, the nucleic acid encodes an antibody comprising a VH comprising the amino acid set forth in SEQ ID NO: 33 and a VL comprising the amino acid sequence set forth in SEQ ID NO:34.

In some embodiments, the nucleic acid encodes an antibody that binds to a peptide of an N-terminally ubiquitinated polypeptide, wherein the antibody comprises one, two, three, four, five, or six CDRs of antibody 1C7 as shown in Table 2A and Table 2B. In some embodiments, the nucleic acid encodes an antibody comprising the VH and/or the VL of antibody 1C7 as shown in Table 3. In some embodiments, the nucleic acid encodes an antibody comprising the heavy chain and/or the light chain of antibody 1C7 as shown in Table 4.

In some embodiments, the nucleic acid encodes an antibody that binds a peptide of an N-terminally ubiquitinated polypeptide, wherein the antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:1. In certain embodiments, the nucleic acid encodes a VH sequence containing substitutions (e.g., conservative substitutions), insertions, or deletions relative to the amino acid sequence of SEQ ID NO:1, but retaining the ability to bind an N-terminally ubiquitinated polypeptide as the antibody comprising SEQ ID NO:1. In certain embodiments, a total of 1 to 13 amino acids have been substituted, inserted and/or deleted in SEQ ID NO: 1. In certain embodiments, substitutions, insertions, or deletions occur in regions outside the CDRs (i.e., in the FRs). In a particular embodiment, the nucleic acid encodes an antibody comprising a VH comprising one, two or three CDRs selected from the group consisting of: (a) a CDRH1 comprising the amino acid sequence of SEQ ID NO:3, (b) a CDRH2 comprising the amino acid sequence of SEQ ID NO:4, and (c) a CDRH3 comprising the amino acid sequence of SEQ ID NO:5.

In another aspect, the nucleic acid encodes an antibody that binds a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:2. In certain embodiments, the nucleic acid encodes an antibody comprising a VL sequence containing substitutions (e.g., conservative substitutions), insertions, or deletions relative to the amino acid sequence of SEQ ID NO:2, but retaining the ability to bind an N-terminally ubiquitinated polypeptide as the antibody comprising SEQ ID NO:2. In certain embodiments, a total of 1 to 11 amino acids have been substituted, inserted and/or deleted in SEQ ID NO:2. In certain embodiments, the substitutions, insertions, or deletions occur in regions outside the CDRs (i.e., in the FRs). In a particular embodiment, the nucleic acid encodes an antibody comprising a VL comprising one, two or three CDRs selected from the group consisting of (a) a CDRL1 comprising the amino acid sequence of SEQ ID NO:6; (b) a CDRL2 comprising the amino acid sequence of SEQ ID NO:7; and (c) a CDRL3 comprising the amino acid sequence of SEQ ID NO:8.

In one embodiment, the nucleic acid encodes an antibody that binds to a peptide of an N-terminally ubiquitinated polypeptide, wherein the antibody comprises a VL comprising the amino acid sequence of SEQ ID NO:2 and a VH comprising the amino acid sequence of SEQ ID NO:1.

In another aspect, a nucleic acid encoding an antibody that binds to a peptide of an N-terminally ubiquitinated polypeptide is provided, wherein the antibody comprises a VH comprising a CDRH1 comprising the amino acid sequence of SEQ ID NO:3, a CDRH2 comprising the amino acid sequence of SEQ ID NO:4, and a CDRH3 comprising the amino acid sequence of SEQ ID NO:5; and a VL comprising a CDRL1 comprising the amino acid sequence of SEQ ID NO:6, a CDRL2 comprising the amino acid sequence of SEQ ID NO:7, and a CDRL3 comprising the amino acid sequence of SEQ ID NO:8.

In another aspect, a nucleic acid encoding an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:1; and a VL CDR1, a VL CDR2, and a VL CDR3, respectively comprising the amino acid sequences of a CDR1, a CDR2, and a CDR3 of a VL having the sequence set forth in SEQ ID NO:2.

In some embodiments, the nucleic acid encodes an antibody comprising a heavy chain having at least 85%, 90%, 91%, 92%, 93%, 94%, 95%, 96%, 97%, 98%, 99%, or 100% sequence identity to the amino acid sequence of SEQ ID NO:52. In certain embodiments, the heavy chain sequence contains substitutions (e.g., conservative substitutions), insertions, or deletions relative to the amino acid sequence of SEQ ID NO:52, but retains the ability to bind an N-terminally ubiquitinated polypeptide as the antibody comprising SEQ ID NO:52. In certain embodiments, a total of 1 to 20 amino acids have been substituted, inserted and/or deleted in SEQ ID NO:52. In some embodiments, the antibody comprises a heavy chain comprising the amino acid sequence set forth in SEQ ID NO: 52.

In some embodiments, the nucleic acid encodes an antibody comprising a light chain having at least 85%, 90%, 91%, 92%, 93%, 94%, 95%, 96%, 97%, 98%, 99%, or 100% sequence identity to the amino acid sequence of SEQ ID NO:53. In certain embodiments, the light chain sequence contains substitutions (e.g., conservative substitutions), insertions, or deletions relative to the amino acid sequence of SEQ ID NO:53, but retains the ability to bind an N-terminally ubiquitinated polypeptide as the antibody comprising SEQ ID NO:53. In certain embodiments, a total of 1 to 20 amino acids have been substituted, inserted and/or deleted in SEQ ID NO:53. In some embodiments, the antibody comprises a light chain comprising the amino acid sequence set forth in SEQ ID NO: 53.

In some embodiments, the nucleic acid encodes an antibody that binds to a peptide of an N-terminally ubiquitinated polypeptide, wherein the antibody comprises one, two, three, four, five, or six CDRs of antibody 2B12 as shown in Table 2A and Table 2B. In some embodiments, the nucleic acid encodes an antibody comprising the VH and/or the VL of antibody 2B12 as shown in Table 3. In some embodiments, the nucleic acid encodes an antibody comprising the heavy chain and/or the light chain of antibody 2B12 as shown in Table 4.

In some embodiments, the nucleic acid encodes an antibody that binds a peptide of an N-terminally ubiquitinated polypeptide, wherein the antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:9. In certain embodiments, the nucleic acid encodes an antibody comprising a VH sequence containing substitutions (e.g., conservative substitutions), insertions, or deletions relative to the amino acid sequence of SEQ ID NO:9, but retaining the ability to bind a peptide of an N-terminally ubiquitinated polypeptide as the antibody comprising SEQ ID NO:9. In certain embodiments, a total of 1 to 13 amino acids have been substituted, inserted and/or deleted in SEQ ID NO:9. In certain embodiments, substitutions, insertions, or deletions occur in regions outside the CDRs (i.e., in the FRs). In a particular embodiment, the nucleic acid encodes an antibody comprising a VH comprising one, two or three CDRs selected from the group consisting of: (a) a CDRH1 comprising the amino acid sequence of SEQ ID NO:11, (b) a CDRH2 comprising the amino acid sequence of SEQ ID NO:12, and (c) a CDRH3 comprising the amino acid sequence of SEQ ID NO:13.

In another aspect, a nucleic acid encoding an antibody that binds a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:10. In certain embodiments, the nucleic acid encodes an antibody comprising a VL sequence containing substitutions (e.g., conservative substitutions), insertions, or deletions relative to the amino acid sequence of SEQ ID NO:10, but retaining the ability to bind a peptide of an N-terminally ubiquitinated polypeptide as the antibody comprising SEQ ID NO:10. In certain embodiments, a total of 1 to 11 amino acids have been substituted, inserted and/or deleted in SEQ ID NO:10. In certain embodiments, the substitutions, insertions, or deletions occur in regions outside the CDRs (i.e., in the FRs). In a particular embodiment, the nucleic acid encodes an antibody comprising a VL comprising one, two or three CDRs selected from the group consisting of (a) a CDRL1 comprising the amino acid sequence of SEQ ID NO:14; (b) a CDRL2 comprising the amino acid sequence of SEQ ID NO:15; and (c) a CDRL3 comprising the amino acid sequence of SEQ ID NO:16.

In one embodiment, the nucleic acid encodes an antibody that binds to a peptide of an N-terminally ubiquitinated polypeptide, wherein the antibody comprises a VL comprising the amino acid sequence of SEQ ID NO:10 and a VH comprising the amino acid sequence of SEQ ID NO:9.

In another aspect, a nucleic acid encoding an antibody that binds to a peptide of an N-terminally ubiquitinated polypeptide is provided, wherein the antibody comprises a VH comprising a CDRH1 comprising the amino acid sequence of SEQ ID NO:11, a CDRH2 comprising the amino acid sequence of SEQ ID NO:12, and a CDRH3 comprising the amino acid sequence of SEQ ID NO:13; and a VL comprising a CDRL1 comprising the amino acid sequence of SEQ ID NO:14, a CDRL2 comprising the amino acid sequence of SEQ ID NO:15, and a CDRL3 comprising the amino acid sequence of SEQ ID NO:16.

In another aspect, a nucleic acid encoding an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:9; and a VL CDR1, a VL CDR2, and a VL CDR3, respectively comprising the amino acid sequences of a CDR1, a CDR2, and a CDR3 of a VL having the sequence set forth in SEQ ID NO:10.

In some embodiments, the nucleic acid encodes an antibody comprising a heavy chain having at least 85%, 90%, 91%, 92%, 93%, 94%, 95%, 96%, 97%, 98%, 99%, or 100% sequence identity to the amino acid sequence of SEQ ID NO:54. In certain embodiments, the heavy chain sequence contains substitutions (e.g., conservative substitutions), insertions, or deletions relative to the amino acid sequence of SEQ ID NO:54, but retains the ability to bind an N-terminally ubiquitinated polypeptide as the antibody comprising SEQ ID NO:54. In certain embodiments, a total of 1 to 20 amino acids have been substituted, inserted and/or deleted in SEQ ID NO:54. In some embodiments, the antibody comprises a heavy chain comprising the amino acid sequence set forth in SEQ ID NO: 54.

In some embodiments, the nucleic acid encodes an antibody comprising a light chain having at least 85%, 90%, 91%, 92%, 93%, 94%, 95%, 96%, 97%, 98%, 99%, or 100% sequence identity to the amino acid sequence of SEQ ID NO:55. In certain embodiments, the light chain sequence contains substitutions (e.g., conservative substitutions), insertions, or deletions relative to the amino acid sequence of SEQ ID NO:55, but retains the ability to bind an N-terminally ubiquitinated polypeptide as the antibody comprising SEQ ID NO:55. In certain embodiments, a total of 1 to 20 amino acids have been substituted, inserted and/or deleted in SEQ ID NO:55. In some embodiments, the antibody comprises a light chain comprising the amino acid sequence set forth in SEQ ID NO: 55.

In some embodiments, the nucleic acid encodes an antibody that binds to a peptide of an N-terminally ubiquitinated polypeptide, wherein the antibody comprises one, two, three, four, five, or six CDRs of antibody 2E9 as shown in Table 2A and Table 2B. In some embodiments, the nucleic acid encodes an antibody comprising the VH and/or the VL of antibody 2E9 as shown in Table 3. In some embodiments, the nucleic acid encodes an antibody comprising the heavy chain and/or the light chain of antibody 2E9 as shown in Table 4.

In some embodiments, the nucleic acid encodes an antibody that binds a peptide of an N-terminally ubiquitinated polypeptide, wherein the antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:17. In certain embodiments, the nucleic acid encodes an antibody comprising a VH sequence containing substitutions (e.g., conservative substitutions), insertions, or deletions relative to the amino acid sequence of SEQ ID NO:17, but retaining the ability to bind a peptide of an N-terminally ubiquitinated polypeptide as the antibody comprising SEQ ID NO:17. In certain embodiments, a total of 1 to 13 amino acids have been substituted, inserted and/or deleted in SEQ ID NO:17. In certain embodiments, substitutions, insertions, or deletions occur in regions outside the CDRs (i.e., in the FRs). In a particular embodiment, the nucleic acid encodes an antibody comprising a VH comprising one, two or three CDRs selected from the group consisting of: (a) a CDRH1 comprising the amino acid sequence of SEQ ID NO:19, (b) a CDRH2 comprising the amino acid sequence of SEQ ID NO:20, and (c) a CDRH3 comprising the amino acid sequence of SEQ ID NO:21.

In another aspect, a nucleic acid encoding an antibody that binds a peptide of a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:18. In certain embodiments, the nucleic acid encodes an antibody comprising a VL sequence containing substitutions (e.g., conservative substitutions), insertions, or deletions relative to the amino acid sequence of SEQ ID NO:18, but retaining the ability to bind an N-terminally ubiquitinated polypeptide as the antibody comprising SEQ ID NO:18. In certain embodiments, a total of 1 to 11 amino acids have been substituted, inserted and/or deleted in SEQ ID NO:18. In certain embodiments, the substitutions, insertions, or deletions occur in regions outside the CDRs (i.e., in the FRs). In a particular embodiment, the nucleic acid encodes an antibody comprising a VL comprising one, two or three CDRs selected from the group consisting of (a) a CDRL1 comprising the amino acid sequence of SEQ ID NO:22; (b) a CDRL2 comprising the amino acid sequence of SEQ ID NO:23; and (c) a CDRL3 comprising the amino acid sequence of SEQ ID NO:24.

In one embodiment, the nucleic acid encodes an antibody that binds to a peptide of an N-terminally ubiquitinated polypeptide, wherein the antibody comprises a VL comprising the amino acid sequence of SEQ ID NO:18 and a VH comprising the amino acid sequence of SEQ ID NO:17.

In another aspect, a nucleic acid encoding an antibody that binds to a peptide of an N-terminally ubiquitinated polypeptide is provided, wherein the antibody comprises a VH comprising a CDRH1 comprising the amino acid sequence of SEQ ID NO:19, a CDRH2 comprising the amino acid sequence of SEQ ID NO:20, and a CDRH3 comprising the amino acid sequence of SEQ ID NO:21; and a VL comprising a CDRL1 comprising the amino acid sequence of SEQ ID NO:22, a CDRL2 comprising the amino acid sequence of SEQ ID NO:23, and a CDRL3 comprising the amino acid sequence of SEQ ID NO:24.

In another aspect, a nucleic acid encoding an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:17; and a VL CDR1, a VL CDR2, and a VL CDR3, respectively comprising the amino acid sequences of a CDR1, a CDR2, and a CDR3 of a VL having the sequence set forth in SEQ ID NO:18.

In some embodiments, the nucleic acid encodes an antibody comprising a heavy chain having at least 85%, 90%, 91%, 92%, 93%, 94%, 95%, 96%, 97%, 98%, 99%, or 100% sequence identity to the amino acid sequence of SEQ ID NO:56. In certain embodiments, the heavy chain sequence contains substitutions (e.g., conservative substitutions), insertions, or deletions relative to the amino acid sequence of SEQ ID NO:56, but retains the ability to bind an N-terminally ubiquitinated polypeptide as the antibody comprising SEQ ID NO:52. In certain embodiments, a total of 1 to 20 amino acids have been substituted, inserted and/or deleted in SEQ ID NO:56. In some embodiments, the antibody comprises a heavy chain comprising the amino acid sequence set forth in SEQ ID NO: 56.

In some embodiments, the nucleic acid encodes an antibody comprising a light chain having at least 85%, 90%, 91%, 92%, 93%, 94%, 95%, 96%, 97%, 98%, 99%, or 100% sequence identity to the amino acid sequence of SEQ ID NO:57. In certain embodiments, the light chain sequence contains substitutions (e.g., conservative substitutions), insertions, or deletions relative to the amino acid sequence of SEQ ID NO:57, but retains the ability to bind an N-terminally ubiquitinated polypeptide as the antibody comprising SEQ ID NO:57. In certain embodiments, a total of 1 to 20 amino acids have been substituted, inserted and/or deleted in SEQ ID NO:57. In some embodiments, the antibody comprises a light chain comprising the amino acid sequence set forth in SEQ ID NO: 57.

In some embodiments, the nucleic acid encodes an antibody that binds to a peptide of an N-terminally ubiquitinated polypeptide, wherein the antibody comprises one, two, three, four, five, or six CDRs of antibody 2H2 as shown in Table 2A and Table 2B. In some embodiments, the nucleic acid encodes an antibody comprising the VH and/or the VL of antibody 2H2 as shown in Table 3. In some embodiments, the nucleic acid encodes an antibody comprising the heavy chain and/or the light chain of antibody 2H2 as shown in Table 4.

In some embodiments, the nucleic acid encodes an antibody that binds a peptide of a peptide of an N-terminally ubiquitinated polypeptide comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO:25. In certain embodiments, the nucleic acid encodes an antibody comprising a VH sequence containing substitutions (e.g., conservative substitutions), insertions, or deletions relative to the amino acid sequence of SEQ ID NO:25, but retaining the ability to bind a peptide of an N-terminally ubiquitinated polypeptide as the antibody comprising SEQ ID NO:25. In certain embodiments, a total of 1 to 13 amino acids have been substituted, inserted and/or deleted in SEQ ID NO:25. In certain embodiments, substitutions, insertions, or deletions occur in regions outside the CDRs (i.e., in the FRs). In a particular embodiment, the nucleic acid encodes an antibody comprising a VH comprising one, two or three CDRs selected from the group consisting of: (a) a CDRH1 comprising the amino acid sequence of SEQ ID NO:27, (b) a CDRH2 comprising the amino acid sequence of SEQ ID NO:28, and (c) a CDRH3 comprising the amino acid sequence of SEQ ID NO:29.

In another aspect, a nucleic acid encoding an antibody that binds a peptide of an N-terminally ubiquitinated polypeptide is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:26. In certain embodiments, the nucleic acid encodes an antibody comprising a VL sequence containing substitutions (e.g., conservative substitutions), insertions, or deletions relative to the amino acid sequence of SEQ ID NO:26, but retaining the ability to bind a peptide of an N-terminally ubiquitinated polypeptide as the antibody comprising SEQ ID NO:26. In certain embodiments, a total of 1 to 11 amino acids have been substituted, inserted and/or deleted in SEQ ID NO:26. In certain embodiments, the substitutions, insertions, or deletions occur in regions outside the CDRs (i.e., in the FRs). In a particular embodiment, the nucleic acid encodes an antibody comprising a VL comprising one, two or three CDRs selected from the group consisting of (a) a CDRL1 comprising the amino acid sequence of SEQ ID NO:30; (b) a CDRL2 comprising the amino acid sequence of SEQ ID NO:31; and (c) a CDRL3 comprising the amino acid sequence of SEQ ID NO:32.

In one embodiment, the nucleic acid encodes an antibody that binds to a peptide of an N-terminally ubiquitinated polypeptide, wherein the antibody comprises a VL comprising the amino acid sequence of SEQ ID NO:26, and a VH comprising the amino acid sequence of SEQ ID NO:25.

In another aspect, a nucleic acid encoding an antibody that binds to a peptide of an N-terminally ubiquitinated polypeptide is provided, wherein the antibody comprises a VH comprising a CDRH1 comprising the amino acid sequence of SEQ ID NO:27, a CDRH2 comprising the amino acid sequence of SEQ ID NO:28, and a CDRH3 comprising the amino acid sequence of SEQ ID NO:29; and a VL comprising a CDRL1 comprising the amino acid sequence of SEQ ID NO:30, a CDRL2 comprising the amino acid sequence of SEQ ID NO:31, and a CDRL3 comprising the amino acid sequence of SEQ ID NO:32.

In another aspect, a nucleic acid encoding an antibody that binds to a peptide of an N-terminally ubiquitinated polypeptide is provided, wherein the antibody comprises a VH CDR1, a VH CDR2, and a VH CDR3, respectively comprising the amino acid sequences of a CDR1, a CDR2, and a CDR3 of a VH having the sequence set forth in SEQ ID NO:25; and a VL CDR1, a VL CDR2, and a VL CDR3, respectively comprising the amino acid sequences of a CDR1, a CDR2, and a CDR3 of a VL having the sequence set forth in SEQ ID NO:26.

In some embodiments, the nucleic acid encodes an antibody comprising a heavy chain having at least 85%, 90%, 91%, 92%, 93%, 94%, 95%, 96%, 97%, 98%, 99%, or 100% sequence identity to the amino acid sequence of SEQ ID NO:58. In certain embodiments, the heavy chain sequence contains substitutions (e.g., conservative substitutions), insertions, or deletions relative to the amino acid sequence of SEQ ID NO:58, but retains the ability to bind an N-terminally ubiquitinated polypeptide as the antibody comprising SEQ ID NO:58. In certain embodiments, a total of 1 to 20 amino acids have been substituted, inserted and/or deleted in SEQ ID NO:58. In some embodiments, the antibody comprises a heavy chain comprising the amino acid sequence set forth in SEQ ID NO: 58.

In some embodiments, the nucleic acid encodes an antibody comprising a light chain having at least 85%, 90%, 91%, 92%, 93%, 94%, 95%, 96%, 97%, 98%, 99%, or 100% sequence identity to the amino acid sequence of SEQ ID NO:59. In certain embodiments, the light chain sequence contains substitutions (e.g., conservative substitutions), insertions, or deletions relative to the amino acid sequence of SEQ ID NO:59, but retains the ability to bind an N-terminally ubiquitinated polypeptide as the antibody comprising SEQ ID NO:59. In certain embodiments, a total of 1 to 20 amino acids have been substituted, inserted and/or deleted in SEQ ID NO:59. In some embodiments, the antibody comprises a light chain comprising the amino acid sequence set forth in SEQ ID NO: 59.

In another aspect, a nucleic acid encoding an antibody that binds to a peptide of an N-terminally ubiquitinated polypeptide is provided, wherein the antibody comprises a VH as in any of the embodiments provided above, and a VL as in any of the embodiments provided above.

Also provided herein is a vector comprising any one of the nucleic acids described herein. Also provided herein is a host cell comprising the vector, and/or any one of the nucleic acids described herein. In some embodiments, the host cell is isolated or purified. In some embodiments, the host cell is in a cell culture medium.

For antibody production, vectors comprising the nucleic acid described herein may be introduced into appropriate production cell lines know in the art such as, for example, NS0 cells. Introduction of the expression vectors may be accomplished by co-transfection via electroporation or any other suitable transformation technology available in the art. Antibody producing cell lines can then be selected and expanded and humanized antibodies purified. The purified antibodies can then be analyzed by standard techniques such as SDS-PAGE.

Also provided is a host cell comprising a nucleic acid encoding an antibody that binds to a peptide of an N-terminally ubiquitinated polypeptide. In some embodiments, the host cell comprises nucleic acid encoding any of the antibodies described herein. Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CVl line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CVl); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

Monoclonal antibodies (including the antibodies that bind to the amino acid sequence GGX at the N-terminus of the peptide, wherein the antibody does not bind to an amino acid sequence comprising a branched diglycine (K-ε-GG), as described herein) may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, California USA, and SP-2 or X63-Ag8-653 cells available from the American Type Culture Collection, Rockville, Maryland USA. Human myeloma and mouse-human heteromyeloma cell lines also have been described for the production of human monoclonal antibodies (Kozbor, J. Immunol., 133:3001 (1984); Brodeur et al., Monoclonal Antibody production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

DNA encoding the antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

The DNA also may be modified, for example, by substituting the coding sequence for human heavy- and light-chain constant domains in place of the homologous murine sequences (U.S. Pat. No. 4,816,567; Morrison, et al., proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

C. Methods of Screening

Also provided herein is a method of screening for an antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG). In some embodiments, X is any amino acid.

In some embodiments, the method comprises i) providing an antibody library; ii) positively selecting antibodies that bind to a peptide comprising the amino acid sequence GGX at the N-terminus, wherein X is any amino acid; and iii) negatively selecting antibodies that bind to a peptide comprising the amino acid sequence K-ε-GG, thereby producing an antibody that specifically binds to a peptide comprising the amino acid sequence GGX at the N-terminus, and does not bind to the amino acid sequence K-ε-GG. In some embodiments, in step ii) antibodies that bind to a peptide comprising the amino acid sequence GGM at the N-terminus are positively selected. In some embodiments, in step ii) antibodies that bind to a peptide comprising an amino acid sequence selected from the group consisting of GGA, GGE, GGF, GGG, GGH, GGI, GGL, GGM, GGN, GGQ, GGS, GGT, GGV, and GGW at the N-terminus are positively selected.

In some embodiments, the method comprises providing an antibody library and positively selecting antibodies that bind to the amino acid sequence GGX at the N-terminus of a peptide of an N-terminally ubiquitinated polypeptide. In some embodiments, a phage display library is provided. In some embodiments, a yeast display library is provided. In some embodiments, a bacterial display library is provided.

The antibody libraries provided herein may comprise antibodies from various sources. For example in some embodiments, a library of synthetic antibodies is provided. In some embodiments, a library of human naïve antibodies is provided. In some embodiments, a library of camel antibodies is provided. In some embodiments, a murine antibody library is provided. In some embodiments, a library of rabbit antibodies is provided. In some embodiments, a library of humanized antibodies is provided.

In some embodiments, the library is produced by immunizing a mammal with a peptide library comprising peptides comprising the amino acid sequence GGM at the N-terminus. In some embodiments, the library is produced by cloning antibodies from an immunized mammal. In some embodiments, the immunized mammal is a rodent (e.g., a mouse) or a rabbit. In some embodiments, the mammal is immunized with a peptide library. In some embodiments, the mammal is immunized with a library of N-terminally ubiquitinated polypeptides. In some embodiments, the mammal is immunized with N-terminally ubiquitinated polypeptides comprising peptides comprising an amino acid sequence GGX at the N-terminus of the peptide. In some embodiments, the mammal is immunized with N-terminally ubiquitinated polypeptides comprising peptides comprising an amino acid sequence GGM at the N-terminus of the peptide

Also provided herein is a peptide library which can be used for producing and/or screening for antibodies that bind to the amino acid sequence GGX at the N-terminus of a peptide of an N-terminally ubiquitinated polypeptide. In some embodiments, X is any amino acid.

In some embodiments, the antibody library is positively selected for antibodies that bind to a peptide comprising the amino acid sequence GGM at the N-terminus. In some embodiments, the antibody library is positively selected by phage panning. In some embodiments, the antibody library is incubated with one or more peptides comprising the amino acid sequence GGM at the N-terminus bound to a solid support. In some embodiments, the unbound antibodies are removed by washing and the bound antibodies are eluted with HCl. In some embodiments, the library is positively selected as least twice, at least three times, at least four times, or more than five times. In some embodiments, the antibody library is positively selected according to a method as described in Example 1 (see, e.g., Example 1, Materials and Methods, Phage library generation and selections).

In some embodiments, the antibody library is positively selected by incubating with one or more N-terminally ubiquitinated polypeptides. In some embodiments, the antibody library is positively selected by incubating with one or more peptides comprising the amino acid sequence GGM at the N-terminus.

In some embodiments, multiple rounds of positive selection are performed with different peptides in each round. In some embodiments, multiple rounds of positive selection are performed with the same peptides in each round.

In some embodiments, the antibody library is negatively selected for antibodies that bind to a peptide comprising the amino acid sequence K-ε-GG. In some embodiments, the negative selection comprises incubating the antibody library with peptides comprising the amino acid sequence K-ε-GG. In some embodiments, the negative selection comprises incubating the antibody library with peptides comprising the amino acid sequence K-ε-GG that are bound to a solid substrate and retaining the supernatant and discarding the bound antibodies. In some embodiments, the negative selection comprises incubating the antibody library with free peptides comprising the amino acid sequence K-ε-GG. In some embodiments, negative selection is performed according to a method as described in Example 1 (see, e.g., Example 1, Materials and Methods, Phage library generation and selections).

In some embodiments, the positive and negative selection are simultaneous. In some embodiments, the antibody library is incubated with one or more peptides comprising the amino acid sequence GGM at the N-terminus bound to a solid support, and incubated with one or more unbound peptides comprising the amino acid sequence K-ε-GG. In some embodiments, antibodies bound to the solid substrate are selected.

In some embodiments, the positive and negative selection are simultaneous. In some embodiments, the antibody library is incubated with one or more unbound peptides comprising the amino acid sequence GGM at the N-terminus, and incubated with one or more peptides comprising the amino acid sequence K-ε-GG bound to a solid support. In some embodiments, antibodies not bound to the solid substrate are selected.

In some embodiments, the positive and negative selection are sequential. For example, in some embodiments, the antibody library is first positively selected for antibodies that bind to peptides comprising the amino acid sequence GGM at the N-terminus, and then negatively selected for antibodies that bind to peptides comprising the amino acid sequence K-ε-GG. In some embodiments, the antibody library is first negatively selected for antibodies that bind to peptides comprising the amino acid sequence K-ε-GG, and then positively selected for antibodies that bind to peptides comprising the amino acid sequence GGM at the N-terminus.

In some embodiments, steps the positive and negative selection are repeated two or more times. For example, in some embodiments, the positive and negative selection are repeated at least two times, at least three times, at least four times, or at least five times.

In some embodiments, selected antibodies are assayed to confirm that they bind to peptides comprising the amino acid sequence GGX at the N-terminus of the peptide, but not an amino acid sequence comprising K-ε-GG. In some embodiments, the antibodies are assayed using ELISA or SPR. In some embodiments, the antibodies are assayed according to a method as described in Example 1 (see, e.g., Example 1, Materials and Methods, pAb ELISAs and Monoclonal antibody ELISAs). In some embodiments, the antibodies are assayed in a ubiquitination assay. An exemplary ubiquitination assay method is described in Example 5.

Also provided herein is an antibody produced by the methods of screening as described herein.

D. Methods of Enriching N-Terminally Ubiquitinated Peptides in a Sample

Also provided herein is method of enriching N-terminally ubiquitinated peptides in a sample comprising a mixture of peptides. Further, libraries of N-terminally ubiquitinated peptides are provided.

1. Methods of Enriching

Provided herein are methods of enriching N-terminally ubiquitinated peptides in a sample comprising a mixture of peptides. In some embodiments, the method comprises i) contacting the sample with an antibody that binds to a peptide of an N-terminally ubiquitinated protein; and ii) selecting antibody-bound peptides from the sample, wherein the antibody binds to the amino acid sequence GGX at the N-terminus, wherein the antibody does not bind to an amino acid sequence comprising a branched diglycine (K-ε-GG). In some embodiments, X is any amino acid. In some embodiments, the antibody is any one of the antibodies that bind to a peptide of an N-terminally ubiquitinated polypeptide, as described herein. In some embodiments, one or more antibodies are used, e.g., an equimolar mixture of antibodies. In some embodiments, an equimolar mixture of 1C7, 2B12, 2E9, and 2H2 is used.

In some embodiments, the sample is a cell lysate. In some embodiments, the sample is a human cell lysate. In some embodiments, the sample is a HEK293 cell lysate. In some embodiments, the sample is a cell lysate derived from a HEK293 cell with inducible ubiquitin-conjugating enzyme E2 (UBE2W) expression. In some embodiments, the sample is a cell lysate derived from a HEK293 cell with inducible UBE2W expression and inducible RNF4 expression.

In some embodiments, the method further comprises deleting a deubiquitinase in a cell and lysing the cell to produce the cell lysate (e.g., by knocking out a gene encoding a deubiquitinase). In some embodiments, the method further comprises downregulating a deubiquitinase in a cell and lysing the cell to produce the cell lysate. In some embodiments, the deubiquitinase is UCHL1 or UCHL5. Without wishing to be bound by theory, it is believed that deleting or downregulating a deubiquitinase increases the number of N-terminal Ub sites.

In some embodiments, the method further comprises overexpressing a ubiquitin ligase in a cell and lysing the cell to produce the cell lysate. In some embodiments, the ubiquitin ligase is an N-terminal ubiquitin ligase. In some embodiments, the ubiquitin ligase is ubiquitin-conjugating enzyme E2 (UBE2W). In some embodiments, overexpression of the ubiquitin ligase in the cell is achieved using a doxycycline (Dox)-inducible expression system. In some embodiments, overexpression of the ubiquitin ligase in the cell is achieved according to a method as described in Example 4 (see, e.g., Example 4, Materials and Methods).

In some embodiments, the cell lysate is incubated with a protease to generate the peptides. In some embodiments, the protease is trypsin. Trypsin is a serine protease that cleaves polypeptide chains at the carboxyl side of lysine or arginine amino acid residues, except when either residue is followed by a proline residue. Trypsin digestion produces peptides that are of an average size that is appropriate for detection by mass spectrometry (about 700-1500 daltons), and are charged due to the presence of the lysine or arginine residue (see, e.g., Lackay, U. A. et al., J proteome Res. 2013 Dec. 6; 12(12):5558-69). Therefore, trypsin digestion is typically performed before mass spectrometry-based proteomics experiments. In some embodiments in which the cell lysate is incubated with trypsin to generate the peptides, the selected antibody-bound peptides are detected using mass spectrometry.

In some embodiments, the cell lysate is incubated with a bacterial or viral protease to generate the peptides. In some embodiments, the cell lysate is incubated with a viral protease to generate the peptides. In some embodiments, the viral protease is a foot-and-mouth disease virus leader protease. In some embodiments, the viral protease is Lb^{pro}. Lb^{pro} is a foot-and-mouth disease virus leader protease. Use of Lb^{pro} for studying ubiquitination has been described, for example, in Swatek, K. N. et al., Nature 2019 Aug. 1; 572(7770): 533-537, and Swatek, K. N. et al., Protocol Exchange 2019 Aug. 22; 10.21203/rs.2.10850/v1, both of which are hereby incorporated by reference in their entirety. Lb^{pro} specifically cleaves peptide bonds preceding Gly-Gly motifs, such as the C-terminal glycine residues of an attached ubiquitin. Accordingly, digestion with Lb^{pro} incompletely removes ubiquitin from substrates, leaving the signature C-terminal Gly-Gly dipeptide attached to the ubiquitinated residue of the substrates. In some embodiments, the viral protease is an engineered viral protease, e.g., an engineered foot-and-mouth disease virus leader protease. In some embodiments, the engineered viral protease is Lb^{pro}*. As described in Swatek, K. N. et al., Nature 2019 Aug. 1; 572(7770): 533-537, Lb^{pro}* is a variant of Lb^{pro} with a L102W amino acid substitution that exhibits enhanced ubiquitin-cleavage activity. Use of Lb^{pro}/Lb^{pro}* to generate polypeptides with Gly-Gly motifs that indicate sites of ubiquitination has been termed “Ub-clipping.” In some embodiments, the peptides generated by protease cleavage (e.g., using Lb^{pro}/Lb^{pro}*) comprise Gly-Gly residues.

In some embodiments, the cell lysate is incubated with a protease to generate the peptides, wherein the protease specifically cleaves ubiquitinated polypeptides. In some embodiments, the cell lysate is incubated with a protease to generate the peptides, wherein the protease cleaves polypeptides at a peptide bond preceding a Gly-Gly motif. In some embodiments, the protease is Lb^{pro} or Lb^{pro}*. Compared to trypsin, which primarily cleaves peptide chains at the carboxyl side of lysine or arginine amino acid residues, Lb^{pro} and Lb^{pro}* selectively cleave proteins with a greater degree of sequence specificity. Accordingly, it is anticipated that the incubation of such a protease with the cell lysate results in a pool of peptides enriched for peptides from ubiquitinated substrates, relative to incubation with a less-specific protease such as trypsin. In some embodiments, incubation with a protease that specifically cleaves ubiquitinated polypeptides improves the level of enrichment of N-terminally ubiquitinated peptides.

In some embodiments, the method further comprises treating the cell with a proteasome inhibitor or an inhibitor of de-ubiquitination prior to lysate generation and to incubation with the protease (e.g., trypsin, Lb^{pro}, or Lb^{pro}*). In some embodiments, the proteasome inhibitor is selected from the group consisting of lactacystin, disulfiram, epigallocatechin-3-gallate, Marizomib (salinosporamide A), Oprozomib (ONX-0912), delanzomib (CEP-18770), epoxomicin, MG132, beta-hydroxy beta-methylbutyrate, and Bortezomib. In some embodiments, the proteasome inhibitor is Bortezomib.

In some embodiments, the method further comprises detecting the selected antibody-bound peptides. In some embodiments, the antibody-bound peptides are detected by mass spectrometry. Preparation of samples for mass spectrometric analysis can be conducted generally according to known techniques (See, e.g., “Modern protein Chemistry: Practical Aspects”, Howard, G. C. and Brown, W. E., Eds. (2002) CRC Press, Boca Raton, Florida). A variety of mass spectrometry systems capable of high mass accuracy, high sensitivity, and high resolution are known in the art and can be employed in the methods of the invention. The mass analyzers of such mass spectrometers include, but are not limited to, quadrupole (Q), time of flight (TOF), ion trap, magnetic sector or FT-ICR or combinations thereof. The ion source of the mass spectrometer should yield mainly sample molecular ions, or pseudo-molecular ions, and certain characterizable fragment ions. Examples of such ion sources include atmospheric pressure ionization sources, e.g. electrospray ionization (ESI) and atmospheric pressure chemical ionization (APCI) and Matrix Assisted Laser Desorption Ionization (MALDI). ESI and MALDI are the two most commonly employed methods to ionize proteins for mass spectrometric analysis. ESI and APCI are the most commonly used ion source techniques for analysis of small molecules by LC/MS (Lee, M. “LC/MS Applications in Drug Development” (2002) J. Wiley & Sons, New York). In some embodiments, the antibody-bound peptides are detected by liquid chromatography with tandem mass spectrometry (LC-MS/MS). An exemplary method for performing LC-MS/MS is described in Example 3. In some embodiments, antibody-bound peptides are separated by liquid chromatography using a nanoAcquity UPLC (Waters). In some embodiments, following liquid chromatography, the separated peptides are introduced to an Orbitrap Elite™ or Q Exactive™ HF mass spectrometer (ThermoFisher) by electrospray ionization. In some embodiments, the antibody-bound peptides are detected by label free quantitative (LFQ) mass spectrometry. In some embodiments, the antibody-bound peptides are detected by tandem mass tag (TMT) mass spectrometry. In some embodiments, the antibody-bound peptides are detected by protein sequencing.

In some embodiments, the antibody-bound peptides are detected using a secondary antibody that binds to the antibody that binds to a peptide of an N-terminally ubiquitinated protein. In some embodiments, the secondary antibody is an anti-rabbit, anti-rodent, or anti-goat secondary antibody. In some embodiments, the secondary antibody is conjugated to a detectable label.

2. Libraries of N-Terminally Ubiquitinated Peptides

Provided herein are libraries of N-terminally ubiquitinated peptides. In some embodiments, the library is produced by any one of the methods of enrichment described above. In some embodiments, the library comprises one or more of the polypeptides listed in Table 7 or Table 8. In some embodiments, the library comprises one or more peptides derived from the group of polypeptides consisting of human AAAT, human AES, human AIG1, human ARF1, human ARL5B, human BABA2, human BUB3, human C1TC, human C2AIL, human C9J470, human CD81, human CDC45, human DCTP1, human DHRSX, human DMKN, human E2AK1, human EF1B, human F13A, human FA60A, human FBRL, human FLOT1, human GCYB1, human GOT1B, human GPAA1, human HIKES, human HNRPK, human IMPA3, human LAT3, human LAT4, human LRWD1, human MED25, human MFS12, human MIP18, human MMGT1, human MOONR, human NARR, human NDUB6, human NENF, human NOL6, human NOP10, human NUDC, human P121A, human PIGC, human PLBL2, human PRDX1, human PRDX2, human PUR9, human QKI, human RAD21, human RCAS1, human REEP1, human RFA1, human RPB1, human RPB7, human RS29, human RS7, human S11IP, human SGMR1, human T179B, human TAF1, human TCPG, human TF3C4, human TM127, human TMM97, human TMX2, human TSN13, human TSN3, human TTC27, human UBAC1, human UBAC2, human UCHL1, human UCHL5, human VKOR1, human VRK3, human ZDH12, human ZN253, and human ZN672. In some embodiments, the library comprises human DCTP1, human F13A, human HNRPK, human PUR9, human RFA1, human RPB7, human SI1IP, and human UCHL5. In some embodiments, the library comprises a peptide derived from human UCHL1. In some embodiments, the library comprises a peptide derived from human UCHL5. In some embodiments, the library comprises one or more peptides derived from the group of polypeptides consisting of Uniprot Accession Nos. Q15758, Q08117, Q9NVV5, P84077, Q96KC2, Q9NXR7, 043684, P11586, Q96HQ2, C9J470, P60033, 075419, Q9H773, Q8N514, Q6E0U4-8, Q9BQI3, P24534, P00488, Q9NP50, P22087, 075955, Q02153, Q9Y3E0, 043292, Q53FT3, P61978, Q9NX62, 075387, Q8N370, Q9UFC0, Q71SY5, Q6NUT3, Q9Y3D0, Q8N4V1, Q2KHM9, P0DI83, 095139, Q9UMX5, Q9H6R4, Q9NPE3, Q9Y266, Q96HA1, Q92535, Q8NHP8, Q06830, P32119, P31939, Q96PU8, 060216, 000559, Q9H902, P27694, P24928, P62487, P62273, P62081, Q8N1F8, Q99720, Q7Z7N9, P21675, P49368, Q9UKN8, 075204, Q5BJF2, Q9Y320, 095857, 060637, Q6P3X3, Q9BSL1, Q8NBM4, P09936, Q9Y5K5, Q9BQB6, Q8IV63, Q96GR4, 075346, and Q499Z4.

E. Methods of Detecting an N-Terminally Ubiquitinated Peptide in a Sample

Also provided herein is a method of detecting an N-terminally ubiquitinated peptide in a sample comprising a mixture of peptides. In some embodiments, the method comprises i) incubating the sample with an enzyme to generate peptides; ii) contacting the peptides with an antibody that binds to a peptide of an N-terminally ubiquitinated protein, and iii) detecting the N-terminally ubiquitinated peptide, wherein the antibody binds to the amino acid sequence GGX at the N-terminus, wherein the antibody does not bind to an amino acid sequence comprising a branched diglycine (K-ε-GG). In some embodiments, X is any amino acid. In some embodiments, the antibody is any one of the antibodies that bind to a peptide of an N-terminally ubiquitinated polypeptide, as described herein.

In some embodiments, the N-terminally ubiquitinated peptide is detected in a blood sample, plasma sample, serum sample, urine sample, saliva sample, sputum sample, lung effusion sample, or a tissue sample. In some embodiments, the sample is a human sample. In some embodiments, the sample is a cell lysate. In some embodiments, the sample is a HEK293 cell lysate. In some embodiments, the sample is a cell lysate derived from a HEK293 cell with inducible ubiquitin-conjugating enzyme E2 (UBE2W) expression. In some embodiments, the sample is a cell lysate derived from a HEK293 cell with inducible UBE2W expression and inducible RNF4 expression.

In some embodiments, the N-terminally ubiquitinated peptide is detected using a secondary antibody that binds to the antibody that binds to a peptide of an N-terminally ubiquitinated protein. In some embodiments, the secondary antibody is an anti-rabbit, anti-rodent, or anti-goat secondary antibody. In some embodiments, the secondary antibody is conjugated to a detectable label.

In some embodiments, the N-terminally ubiquitinated peptide is detected in a cell lysate. In some embodiments, the method further comprises overexpressing a ubiquitin ligase in a cell and lysing the cell to produce the cell lysate. In some embodiments, the ubiquitin ligase is ubiquitin-conjugating enzyme E2 (UBE2W). In some embodiments, overexpression of the ubiquitin ligase in the cell is achieved using a doxycycline (Dox)-inducible expression system. In some embodiments, overexpression of the ubiquitin ligase in the cell is achieved according to a method as described in Example 4 (see, e.g., Example 4, Materials and Methods). In some embodiments, the method further comprises deleting a deubiquitinase in a cell and lysing the cell to produce the cell lysate (e.g., by knocking out a gene encoding a deubiquitinase). In some embodiments, the method further comprises downregulating a deubiquitinase in a cell and lysing the cell to produce the cell lysate. In some embodiments, the deubiquitinase is UCHL1 or UCHL5. Without wishing to be bound by theory, it is believed that deleting or downregulating a deubiquitinase increases the number of N-terminal Ub sites.

In some embodiments, the cell lysate is incubated with a viral protease to generate the peptides. In some embodiments, the viral protease is a foot-and-mouth disease virus leader protease. In some embodiments, the viral protease is Lb^{pro}. In some embodiments, the viral protease is an engineered viral protease, e.g., an engineered foot-and-mouth disease virus leader protease. In some embodiments, the engineered viral protease is Lb^{pro}*. In some embodiments, the viral protease cleaves polypeptides at a peptide bond preceding a Gly-Gly motif. In some embodiments, the peptides generated by protease cleavage (e.g., using Lb^{pro}/Lb^{pro}*) comprise Gly-Gly residues.

In some embodiments, the method further comprises treating the cell with a proteasome inhibitor or an inhibitor of de-ubiquitination prior lysate generation and to incubation with the viral protease (e.g., Lb^{pro}, or Lb^{pro}*). In some embodiments, the proteasome inhibitor is selected from the group consisting of lactacystin, disulfiram, epigallocatechin-3-gallate, Marizomib (salinosporamide A), Oprozomib (ONX-0912), delanzomib (CEP-18770), epoxomicin, MG132, beta-hydroxy beta-methylbutyrate, and Bortezomib. In some embodiments, the proteasome inhibitor is Bortezomib.

The detection can be carried out by any suitable method, for example, those based on mass spectrometry, immunofluorescent microscopy, flow cytometry, fiber-optic scanning cytometry, or laser scanning cytometry. In some embodiments, the detection is an immunoassay. In some embodiments, the detection is an enzyme linked immunosorbent assay (ELISA) or radioimmunoassay. In some embodiments, the immunoassay comprises immunoblotting, immunodiffusion, immunoelectrophoresis, or immunoprecipitation. In some embodiments, an N-terminally ubiquitinated polypeptide is detected by blotting with an antibody that binds to a peptide of an N-terminally ubiquitinated polypeptide.

F. Kits

The screening, enrichment, and detection methods of this invention can be provided in the form of a kit. In some embodiments, such a kit for screening, enrichment, or detection comprises an antibody that binds to a peptide of an N-terminally ubiquitinated polypeptide or a composition comprising an antibody binds to a peptide of an N-terminally ubiquitinated polypeptide as described herein. The antibody may be any one of the antibodies that bind to a peptide of an N-terminally ubiquitinated polypeptide as described herein. In some embodiments, the antibody comprises a variable heavy chain (VH) and a variable light chain (VL), wherein the antibody comprises a CDRH1 comprising the amino acid sequence XXXMN (SEQ ID NO: 35); a CDRH2 comprising the amino acid sequence XXXXXGXXYYATWA (SEQ ID NO:36); and a CDRH3 comprising the amino acid sequence DDXXXXNX (SEQ ID NO:37); wherein the antibody comprises a CDRL1 comprising the amino acid sequence QSXXSVYXXNXLX (SEQ ID NO:38); a CDRL2 comprising the amino acid sequence XASTLXS (SEQ ID NO: 39); and a CDRL3 comprising the amino acid sequence LGXXDCXSXDCXX (SEQ ID NO:40); wherein X is any amino acid. In some embodiments, the antibody comprises a VH comprising the amino acid set forth in SEQ ID NO: 33. In some embodiments, the antibody comprises a VL comprising the amino acid sequence set forth in SEQ ID NO:34. In some embodiments, the antibody comprises a VH comprising the amino acid set forth in SEQ ID NO: 33 and a VL comprising the amino acid sequence set forth in SEQ ID NO:34. In various embodiments, the antibody that binds to a peptide of an N-terminally ubiquitinated polypeptide is one or more of the antibodies described herein (e.g., 1C7, 2B12, 2E9, or 2H2).

In some embodiments, a kit for use in a method of screening for an antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG), as described herein, is provided. In some embodiments, X is any amino acid. In some embodiments, the kit for use in a method of screening for an antibody that binds to a peptide of an N-terminally ubiquitinated polypeptide comprises any of the antibodies that bind to a peptide of an N-terminally ubiquitinated polypeptide, as described herein (e.g., 1C7, 2B12, 2E9, or 2H2). In some embodiments, the kit for use in a method of screening for an antibody that binds to a peptide of an N-terminally ubiquitinated polypeptide provides instructions for performing positive selection (e.g., selection for binding an N-terminally ubiquitinated polypeptide) or negative selection (e.g., selection for not binding to the amino acid sequence K-ε-GG), as described herein. In some embodiments, the kit for use in a method of screening for an antibody that binds to a peptide of an N-terminally ubiquitinated polypeptide comprises a peptide library which can be used for producing and/or screening for antibodies that bind to a peptide of an N-terminally ubiquitinated polypeptide. In some embodiments, the peptide library comprises peptides comprising the amino acid sequence GGX at the N-terminus (e.g., the amino acid sequence GGM at the N-terminus). In some embodiments, the kit for use in a method of screening for an antibody that binds to a peptide of an N-terminally ubiquitinated polypeptide comprises a peptide library that can be used for negative selection. In some embodiments, the peptide library for negative selection comprises peptides comprising the amino acid sequence K-ε-GG. In some embodiments, the kit for use in a method of screening for an antibody that binds to a peptide of an N-terminally ubiquitinated polypeptide provides a reagent for detecting binding of the antibody to N-terminally ubiquitinated polypeptide. In some embodiments, the kit for use in a method of screening for an antibody that binds to a peptide of an N-terminally ubiquitinated polypeptide provides a reagent for detecting binding of the antibody to the peptide library (e.g., peptides comprising the amino acid sequence GGX at the N-terminus). In some embodiments, the kit for use in a method of screening for an antibody that binds to a peptide of an N-terminally ubiquitinated polypeptide provides a reagent for detecting binding of the antibody to the peptide library for negative selection. In some embodiments, binding of the antibody to the peptide library or the peptide library for negative selection is detected by ELISA. In some embodiments, the kit provides instructions or a reagent for an ELISA. In some embodiments, the kit for use in a method of screening for an antibody that binds to a peptide of an N-terminally ubiquitinated polypeptide comprises an N-terminally ubiquitinated peptide (e.g., UBE2W and/or LUBAC) as a standard.

In some embodiments, a kit for use in a method of enriching N-terminally ubiquitinated peptides, as described herein, is provided. In some embodiments, the kit for use in a method of enriching N-terminally ubiquitinated peptides comprises any of the antibodies that bind to a peptide of an N-terminally ubiquitinated polypeptide, as described herein (e.g., 1C7, 2B12, 2E9, or 2H2). In some embodiments, the kit for use in a method of enriching N-terminally ubiquitinated peptides comprises a reagent for contacting the sample with the antibody. In some embodiments, the reagent for contacting the sample with the antibody is a suitable buffer. In some embodiments, the kit for use in a method of enriching N-terminally ubiquitinated peptides comprises a reagent for selecting antibody-bound peptides from the sample. In some embodiments, the reagent for selecting antibody-bound peptides from the sample is a capture reagent, as described above. In some embodiments, the kit for use in a method of enriching N-terminally ubiquitinated peptides provides instructions for detecting the selected antibody-bound peptides. In some embodiments, the kit for use in a method of enriching N-terminally ubiquitinated peptides provides reagents for detecting the selected antibody-bound peptides. In some embodiments, the antibody-bound peptides are detected by protein sequencing. In some embodiments, the kit for use in a method of enriching N-terminally ubiquitinated peptides provides instructions for protein sequencing. In some embodiments, the kit for use in a method of enriching N-terminally ubiquitinated peptides comprises an N-terminally ubiquitinated peptide (e.g., UBE2W and/or LUBAC) as a standard.

In some embodiments, the kit for use in a method of enriching N-terminally ubiquitinated peptides comprises further comprises a protease (e.g., trypsin, a bacterial protease, or a viral protease). In some embodiments, the protease is Lb^{pro}, or Lb^{pro}*. In some embodiments, the protease cleaves polypeptides at a peptide bond preceding a Gly-Gly motif. In some embodiments, the kit for use in a method of enriching N-terminally ubiquitinated peptides provides reagents and instructions for incubating the protease with the cell lysate, for example, as described in Swatek, K. N. et al., Protocol Exchange 2019 Aug. 22; 10.21203/rs.2.10850/v1. In some embodiments, the kit for use in a method of enriching N-terminally ubiquitinated peptides provides reagents and instructions for “Ub-clipping” the cell lysate. In some embodiments, the kit for use in a method of enriching N-terminally ubiquitinated peptides further comprises reagents and instructions for detecting the N-terminally ubiquitinated peptides, e.g., according to the detection kits described below. In a particular embodiment, the enriched N-terminally ubiquitinated peptides are detected using a western blot. In some embodiments, the kit comprises a secondary antibody.

In one aspect, a kit for detecting N-terminally ubiquitinated peptides in a sample is provided. In some embodiments, the kit for detecting comprises an antibody that binds to a peptide of an N-terminally ubiquitinated polypeptide and instructions for use, wherein the antibody that binds to a peptide of an N-terminally ubiquitinated polypeptide, wherein the antibody binds to the amino acid sequence GGX at the N-terminus, wherein the antibody does not bind to an amino acid sequence comprising a branched diglycine (K-ε-GG). In some embodiments, X is any amino acid. In some embodiments, the kit for detecting N-terminally ubiquitinated peptides provides instructions for detecting an N-terminally ubiquitinated polypeptide with the antibody. In some embodiments, the kit for detecting N-terminally ubiquitinated peptides provides an antibody that binds to a peptide of an N-terminally ubiquitinated polypeptide and a method for detecting the antibody. For example, in some embodiments, the kit for detecting N-terminally ubiquitinated peptides provides an antibody that binds to a peptide of an N-terminally ubiquitinated polypeptide that is conjugated to a label. In some embodiments, the antibody is labeled with biotin, digoxigenin, or fluorescein. In some embodiments, the kit for detecting N-terminally ubiquitinated peptides provides reagents for detecting an N-terminally ubiquitinated polypeptide with the antibody. In some embodiments, the kit for detecting N-terminally ubiquitinated peptides provides reagents for an ELISA to detect an N-terminally ubiquitinated polypeptide with the antibody. In some embodiments, the kit for detecting N-terminally ubiquitinated peptides provides reagents for detecting an N-terminally ubiquitinated polypeptide in a western blot with the antibody. In some embodiments, the kit for detecting N-terminally ubiquitinated peptides provides reagents for an SPR assay to detect an N-terminally ubiquitinated polypeptide with the antibody. In some embodiments, the kit for detecting N-terminally ubiquitinated peptides provides reagents to an N-terminally ubiquitinated polypeptide with the antibody in an immunoprecipitation. In some embodiments, such a kit for detecting N-terminally ubiquitinated peptides is a packaged combination including the basic elements of: a capture reagent comprised of an antibody that binds to a peptide of an N-terminally ubiquitinated polypeptide; a detectable (labeled or unlabeled) antibody that binds to a peptide of an N-terminally ubiquitinated polypeptide as described herein; and instructions on how to perform the assay method using these reagents. These basic elements are defined hereinabove. The kit for detecting N-terminally ubiquitinated peptides may further comprise a solid support for the capture reagents, which may be provided as a separate element or on which the capture reagents are already immobilized. Hence, the capture antibodies in the kit may be immobilized on a solid support, or they may be immobilized on such support that is included with the kit or provided separately from the kit. In some embodiments, the capture reagents are coated on or attached to a solid material (for example, a microtiter plate, beads or a comb). The detectable antibodies may be labeled antibodies detected directly or unlabeled antibodies that are detected by labeled antibodies directed against the unlabeled antibodies raised in a different species. Where the label is an enzyme, the kit will ordinarily include substrates and cofactors required by the enzyme; where the label is a fluorophore, a dye precursor that provides the detectable chromophore; and where the label is biotin, an avidin such as avidin, streptavidin, or streptavidin conjugated to HRP or β-galactosidase with MUG. The kit for detecting N-terminally ubiquitinated peptides also typically contains an N-terminally ubiquitinated peptide (e.g., UBE2W and/or LUBAC) as a standard as well as other additives such as stabilizers, washing and incubation buffers, and the like.

In some embodiments, the antibody that binds to a peptide of an N-terminally ubiquitinated polypeptide is conjugated to a detectable label. In some embodiments, the detectable label is selected from the group consisting of biotin, digoxigenin, and fluorescein. In some embodiments, the antibody is immobilized on a solid support. In some embodiments, the antibody is immobilized on a bead. In some embodiments, the kit for detecting N-terminally ubiquitinated peptides further comprises a protease (e.g., trypsin, a bacterial protease, or a viral protease). In some embodiments, the protease is Lb^{pro} or Lb^{pro}*. In some embodiments, the protease cleaves polypeptides at a peptide bond preceding a Gly-Gly motif. In some embodiments, the kit for detecting N-terminally ubiquitinated peptides comprises instructions for using the protease, e.g., for the digestion of a sample prior to detection using an antibody that binds to a peptide of an N-terminally ubiquitinated polypeptide.

### Embodiments

1. An antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG).
2. The antibody of embodiment 1, wherein the antibody binds to a peptide comprising an N-terminal sequence selected from the group consisting of GGA, GGE, GGF, GGG, GGH, GGI, GGL, GGM, GGN, GGQ, GGS, GGT, GGV, and GGW.
3. The antibody of embodiment 1 or embodiment 2, wherein the antibody binds to a peptide comprising the N-terminal sequence of GGA, a peptide comprising the N-terminal sequence of GGE, a peptide comprising the N-terminal sequence of GGF, a peptide comprising the N-terminal sequence of GGG, a peptide comprising the N-terminal sequence of GGH, a peptide comprising the N-terminal sequence of GGI, a peptide comprising the N-terminal sequence of GGL, a peptide comprising the N-terminal sequence of GGM, a peptide comprising the N-terminal sequence of GGN, a peptide comprising the N-terminal sequence of GGQ, a peptide comprising the N-terminal sequence of GGS, a peptide comprising the N-terminal sequence of GGT, a peptide comprising the N-terminal sequence of GGV, and a peptide comprising the N-terminal sequence of GGW.
4. The antibody of any one of embodiments 1-3, wherein the antibody is a rabbit, rodent, or goat antibody.
5. The antibody of any one of embodiments 1-4, wherein the antibody is a full-length antibody or a Fab fragment.
6. The antibody of any one of embodiments 1-5, wherein the antibody is conjugated to a detectable label.
7. The antibody of embodiment 6, wherein the label is selected from the group consisting of biotin, digoxigenin, and fluorescein.
8. The antibody of any one of embodiments 1-7, wherein the antibody is immobilized on a solid support.
9. The antibody of embodiment 8, wherein the antibody is immobilized on a bead.
10. The antibody of any one of embodiments 1-9, wherein the antibody comprises a variable heavy chain (VH) comprising an Asn at position 35, Val at position 37, Thr at position 93, Asn at position 101, and Trp at position 103 on one side, and a variable light chain (VL) comprising an Ala at position 34, a Tyr at position 36, and a Tyr at position 49, numbering according to Kabat.
11. The antibody of any one of embodiments 1-9, wherein the antibody comprises a variable heavy chain (VH) and a variable light chain (VL), wherein the antibody comprise a CDRH1 comprising the amino acid sequence XXXMN (SEQ ID NO: 35); a CDRH2 comprising the amino acid sequence XXXXXGXXYYATWA (SEQ ID NO:36); and a CDRH3 comprising the amino acid sequence DDXXXXNX (SEQ ID NO:37); wherein the antibody comprises a CDRL1 comprising the amino acid sequence QSXXSVYXXNXLX (SEQ ID NO:38); a CDRL2 comprising the amino acid sequence XASTLXS (SEQ ID NO: 39); and a CDRL3 comprising the amino acid sequence LGXXDCXSXDCXX (SEQ ID NO:40); wherein X is any amino acid.
12. The antibody of embodiment 11, wherein the VH comprises the amino acid set forth in SEQ ID NO: 33 and the VL comprises the amino acid sequence set forth in SEQ ID NO:34.
13. The antibody of any one of embodiments 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 1 and a CDRL1, CDRL2, and CDRL3 of a VL comprising the amino acid sequence set forth in SEQ ID NO: 2.
14. The antibody of embodiment 13, wherein the antibody comprises the CDRH1 amino acid sequence set forth in SEQ ID NO: 3; the CDRH2 amino acid sequence set forth in SEQ ID NO: 4; the CDRH3 amino acid sequence set forth in SEQ ID NO:5; the CDRL1 amino acid sequence set forth in SEQ ID NO: 6; the CDRL2 amino acid sequence set forth in SEQ ID NO:7; and the CDRL3 amino acid sequence set forth in SEQ ID NO:8.
15. The antibody of embodiment 13, wherein the VH comprise the amino acid sequence set forth in SEQ ID NO: 1 and the VL comprises the amino acid sequence set forth in SEQ ID NO:2.
16. The antibody of any one of embodiments 13-15, wherein the antibody comprises a heavy chain and a light chain, wherein the heavy chain comprises the amino acid sequence set forth in SEQ ID NO: 52, and the light chain comprises the amino acid set forth in SEQ ID NO: 53.
17. The antibody of any one of embodiments 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 9 and a CDRL1, CDRL2, and CDRL3 of a VL comprising the amino acid sequences set forth in SEQ ID NO: 10.
18. The antibody of embodiment 17, wherein the antibody comprises the CDRH1 amino acid sequence set forth in SEQ ID NO: 11; the CDRH2 amino acid sequence set forth in SEQ ID NO: 12; the CDRH3 amino acid sequence set forth in SEQ ID NO:13; the CDRL1 amino acid sequence set forth in SEQ ID NO: 14; the CDRL2 amino acid sequence set forth in SEQ ID NO:15; and the CDRL3 amino acid sequence set forth in SEQ ID NO:16.
19. The antibody of embodiment 18, wherein the VH comprises the amino acid sequence set forth in SEQ ID NO: 9 the VL comprises the amino acid sequence set forth in SEQ ID NO:10.
20. The antibody of any one of embodiments 17-19, wherein the antibody comprises a heavy chain and a light chain, wherein the heavy chain comprises the amino acid sequence set forth in SEQ ID NO: 54, and the light chain comprises the amino acid set forth in SEQ ID NO: 55.
21. The antibody of any one of embodiments 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 17 and a CDRL1, CDRL2, and CDRL3 of a VL comprising the amino acid sequence set forth in SEQ ID NO: 18.
22. The antibody of embodiment 21, wherein the antibody comprises the CDRH1 amino acid sequence set forth in SEQ ID NO: 19; the CDRH2 amino acid sequence set forth in SEQ ID NO: 20; the CDRH3 amino acid sequence set forth in SEQ ID NO:21; the CDRL1 amino acid sequence set forth in SEQ ID NO: 22; the CDRL2 amino acid sequence set forth in SEQ ID NO:23; and the CDRL3 amino acid sequence set forth in SEQ ID NO:24.
23. The antibody of embodiment 22, wherein the VH comprises the amino acid set forth in SEQ ID NO: 17 and the VL comprises the amino acid set forth in SEQ ID NO:18.
24. The antibody of any one of embodiments 21-23, wherein the antibody comprises a heavy chain and a light chain, wherein the heavy chain comprises the amino acid sequence set forth in SEQ ID NO: 56, and the light chain comprises the amino acid set forth in SEQ ID NO: 57.
25. The antibody of any one of embodiments 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 25 and a CDRL1, CDRL2, and CDRL3 of a VL comprising the amino acid sequences set forth in SEQ ID NO: 26.
26. The antibody of embodiment 25, wherein the antibody comprises the CDRH1 amino acid sequence set forth in SEQ ID NO: 27; the CDRH2 amino acid sequence set forth in SEQ ID NO: 28; the CDRH3 amino acid sequence set forth in SEQ ID NO:29; a CDRL1 amino acid sequence set forth in SEQ ID NO: 30; the CDRL2 amino acid sequence set forth in SEQ ID NO:31; and the CDRL3 amino acid sequence set forth in SEQ ID NO:32.
27. The antibody of embodiment 26, wherein the VH comprises the amino acid sequence set forth in SEQ ID NO: 25 and the VL comprises the amino acid sequence set forth in SEQ ID NO: 26.
28. The antibody of any one of embodiment 25-27, wherein the antibody comprises a heavy chain and a light chain, wherein the heavy chain comprises the amino acid sequence set forth in SEQ ID NO: 58, and the light chain comprises the amino acid set forth in SEQ ID NO: 59.
29. Nucleic acid encoding the antibody of any one of embodiments 1-28.
30. A host cell comprising the nucleic acid of embodiment 29.
31. A method of screening for an antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG), the method comprising
   i) providing an antibody library;
   ii) positively selecting antibodies that bind to a peptide comprising the amino acid sequence GGX at the N-terminus, wherein X is any amino acid; and
   iii) negatively selecting antibodies that bind to a peptide comprising the amino acid sequence K-ε-GG,
   thereby producing an antibody that specifically binds to a peptide comprising the amino acid GGX at the N-terminus, and does not bind to the amino acid sequence K-ε-GG.
32. The method of embodiment 31, wherein in step ii) antibodies that bind to a peptide comprising the amino acid sequence GGM at the N-terminus are positively selected.
33. The method of claim 31 or 32, wherein negatively selecting antibodies that bind to a peptide comprising the amino acid sequence K-ε-GG is performed simultaneously with step ii).
34. The method of embodiment 31 or 32 wherein negatively selecting antibodies that bind to a peptide comprising the amino acid sequence K-ε-GG before or after step ii).
35. The method of any one of embodiments 31-34, wherein the library is a phage library or a yeast library.
36. The method of any one of embodiments 31-35, wherein the library is produced by immunizing a mammal with a peptide library comprising peptides comprising the amino acid sequence GGM at the N-terminus.
37. The method of embodiment 36 wherein the mammal is a rabbit or a mouse.
38. The method of any one of embodiments 31-37, wherein steps ii)-iii) are repeated two or more times.
39. An antibody produced by the method of any one of embodiments 31-38.
40. A method of enriching N-terminally ubiquitinated peptides in a sample comprising a mixture of peptides, comprising:
   i) contacting the sample with an antibody that binds to a peptide of an N-terminally ubiquitinated protein; and
   ii) selecting antibody-bound peptides from the sample, wherein the antibody binds to the amino acid sequence GGX at the N-terminus, wherein the antibody does not bind to an amino acid sequence comprising a branched diglycine (K-ε-GG).
41. The method of embodiment 40, wherein the sample is a cell lysate.
42. The method of embodiment 41, further comprising deleting a deubiquitinase in a cell and lysing the cell to produce the cell lysate.
43. The method of embodiment 41, further comprising overexpressing a ubiquitin ligase in a cell and lysing the cell to produce the cell lysate.
44. The method of any one of embodiments 41-43, wherein the cell lysate is incubated with trypsin to generate the peptides.
45. The method of any one of embodiments 41-43, wherein the cell lysate is incubated with a bacterial or viral protease to generate the peptides.
46. The method of any one of embodiments 42-45, further comprising treating the cell with a proteasome inhibitor or an inhibitor of de-ubiquitination prior to lysate generation and incubation with trypsin or prior to incubation with the bacterial or viral protease.
47. The method of any one of embodiments 40-46, further comprising detecting the selected antibody-bound peptides.
48. The method of embodiment 47, wherein the antibody-bound peptides are detected by mass spectrometry.
49. The method of embodiment 47, wherein the antibody-bound peptides are detected by protein sequencing.
50. The method of embodiment 47, wherein the antibody-bound peptides are detected using a secondary antibody that binds to the antibody that binds to a peptide of an N-terminally ubiquitinated protein.
51. A library of N-terminally ubiquitinated peptides produced by the method of any one of embodiments 40-50.
52. A method of detecting an N-terminally ubiquitinated peptide in a sample comprising a mixture of peptides comprising
   i) incubating the sample with an enzyme to generate peptides;
   ii) contacting the peptides with an antibody that binds to a peptide of an N-terminally ubiquitinated protein, and
   iii) detecting the N-terminally ubiquitinated peptide, wherein the antibody binds to the amino acid sequence GGX at the N-terminus, wherein the antibody does not bind to an amino acid sequence comprising a branched diglycine (K-ε-GG).
53. The method of embodiment 52, wherein the N-terminally ubiquitinated peptide is detected using a secondary antibody that binds to the antibody that binds to a peptide of an N-terminally ubiquitinated protein.
54. The method of embodiment 52 or 53, wherein the sample is a cell lysate.
55. The method of embodiment 54, further comprising deleting a deubiquitinase in a cell and lysing the cell to produce the cell lysate.
56. The method of 54, further comprising overexpressing a ubiquitin ligase in a cell and lysing the cell to produce the cell lysate.
57. The method of any one of embodiments 54-56, wherein the cell lysate is incubated with a bacterial or viral protease to generate the peptides.
58. The method of any one of embodiments 55-57, further comprising treating the cell with a proteasome inhibitor or an inhibitor of de-ubiquitination prior to lysate generation and incubation with the bacterial or viral protease.
59. A kit for detecting N-terminally ubiquitinated peptides in a sample comprising an antibody that binds to a peptide of an N-terminally ubiquitinated polypeptide and instructions for use, wherein the antibody that binds to a peptide of an N-terminally ubiquitinated polypeptide, wherein the antibody binds to the amino acid sequence GGX at the N-terminus, wherein the antibody does not bind to an amino acid sequence comprising a branched diglycine (K-ε-GG).
60. The kit of embodiment 59, wherein the antibody that binds to a peptide of an N-terminally ubiquitinated polypeptide is conjugated to a detectable label.
61. The kit of embodiment 60, wherein the detectable label is selected from the group consisting of biotin, digoxigenin, and fluorescein.
62. The kit of any one of embodiments 59-61, wherein the antibody is immobilized on a solid support.
63. The kit of embodiment 62, wherein the antibody is immobilized on a bead.
64. The kit of any one of embodiments 59-63, further comprising a protease.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Generation of Novel Anti-GGX Monoclonal Antibodies

The following example describes the generation of antibodies capable of selectively enriching for tryptic peptides containing a diglycine sequence at their N-termini.

### Materials and Methods

### Design of Antibody Selection

A selection was designed to identify antibodies capable of selectively enriching for tryptic peptides containing a diglycine sequence at their N-termini (see FIG. 1A). Without wishing to be bound by theory, it was hypothesized that a sizeable pool of potential substrates would be nascent polypeptides with non-acetylated, intact initiator methionines that, upon trypsin digestion, yield peptides with a diglycine modification prior to the start methionine (Waller, J.-P. J Mol Biol 7, 483-IN1 (1963)). Therefore, a Gly-Gly-Met (GGM) peptide was used as antigen for rabbit immunizations, since rabbits are known to generate high affinity antibodies to peptides and small haptens (Weber, J. et al., Exp Mol Medicine 49, e305-e305 (2017)) (see Rabbit immunizations methods below). Importantly, following the purification of polyclonal antibody (pAb) sera, a selection was performed to identify monoclonal antibodies (mAbs) that possessed minimal cross-reactivity to the conventional and more abundant K-ε-GG peptides, even though they share an identical diglycine sequence feature (see Phage library generation and selections methods below; see GGM and K-ε-GG peptide structures in FIG. 1B).

### Rabbit Immunizations

Eight New Zealand white rabbits were immunized with a Gly-Gly-Met peptide conjugated to either Keyhole limpet haemocyanin (KLH) or ovalbumin (OVA) carrier proteins in order to elicit an immune response in the animal. Rabbits were primed with 500 μg of KLH linked peptide mixed with CFA adjuvant and subsequently injected intradermally. Four biweekly boosts were done with 250 μg of the peptide antigen in IFA adjuvant. To ensure that the B cell response was directed towards the peptide and not the carrier protein, the carrier was alternated with each boost. After the last boost, 5-10 mL of blood was drawn from each rabbit and protein A purified pAb sera was generated to monitor the immune response by enzyme-linked immunosorbent assay (ELISA). The four rabbits with the best titers were euthanized and the spleen and gut associated lymphoid tissue (GALT) were harvested.

### Phage Library Generation and Selections

Total RNA extracted from the rabbit spleen and gut associated lymphoid tissue was used to amplify the variable heavy (VH) and variable light (VL) repertoires separately. Using standard Gibson cloning methods, the VH and VL repertoires were assembled into a single chain Fv (scFv) format and cloned into a phage display vector. The peptide antigens used for selections were either a BSA conjugated or C-terminally biotinylated GGM peptide and a biotinylated K-ε-GG peptide (AAA{K-ε-GG}AAA) for counter-selections. Three rounds of plate-based selections were done in which bound phage was eluted with 100 mM HCl, neutralized, and amplified in E. coli XL1-blue (Stratagene) with the addition of M13-K07 helper phage (New England Biolabs). After selections, individual phage clones were picked and grown in 96-well deep well blocks with 2×YT growth media in the presence of carbenicillin and M13-KO7. After pelleting, the culture supernatants were used in phage ELISAs to screen for specificity.

### pAb ELISAs

Biotinylated GGM or K-ε-GG peptides were coated at 10 μg/mL in PBS on neutravidin ELISA plates (Thermo Scientific) in triplicate overnight at 4° C. Plates were washed with PBS with Tween® 20 (PBST) solution prior to use. Serial dilutions of protein A-purified pAb starting at 100 μg/mL was incubated for 1-2 hours at 25° C. Plates were washed with PBST. After washing, an anti-rabbit Fc-specific HRP 2° antibody (vendor) was added for 1 hour at 25° C. Plates were washed, developed with 3,3′,5,5′-Tetramethylbenzidine (TMB) substrate for 5 minutes, and detected at 650 nm (see FIG. 1C).

### Monoclonal Antibody ELISAs

Biotinylated peptides (GGM and K-ε-GG) were coated at 1 μg/mL in PBS on neutravidin ELISA plates (Thermo Scientific) in triplicate overnight at 4° C. Plates were washed with PBST prior to use. Serial dilutions of GGX mAbs or K-ε-GG mAb (Cell Signaling Technology) at 1 μg/mL were added for 1-2 hours at 25° C. Plates were washed and further developed as described above (see FIG. 1E).

Biotinylated GGX peptides were synthesized and coated at 1 μg/mL in PBS on neutravidin ELISA plates (Thermo Scientific) in triplicate overnight at 4° C. Plates were washed and serial dilutions of GGX mAbs starting at 1 μg/mL were added for 1-2 hours at 25° C. ELISA plates were washed with PBST and developed as described above (see FIG. 1F).

### Fab and IgG Production

Constructs for bacterial expression of Fabs were generated by gene synthesis. Fabs were subsequently expressed and purified as previously described (Simmons, L. C. et al., J Immunol Methods 263, 133-147 (2002); Lombana, T. N. et al., Sci Rep (2015) doi:https://doi.org/10.1038/srep17488). Constructs for mammalian expression of rabbit IgGs were generated by gene synthesis. Plasmids encoding for the LC and HC were co-transfected into 293 cells and purified with affinity chromatography followed by SEC using standard methods (MabSelect SuRe™; GE Healthcare, Piscataway, NJ, USA).

### DNA Constructs

All DNA constructs were obtained by custom gene synthesis (GeneScript) and subcloned into the doxycycline-inducible piggyBac transposon plasmids (BH1.2, Genentech) using the NcoI and XhoI sites.

### Monoclonal Antibody Sequencing

The amino acid sequences of antibodies 1C7, 2B12, 2E9, and 2H2 were determined using techniques standard in the art (see FIG. 1D, and Tables 2-4).

### Results

ELISAs with purified polyclonal antibodies (pAb) confirmed a robust immune response against the GGM peptide, with surprisingly minimal cross reactivity to peptides bearing K-ε-GG (FIG. 1C). Based on the strong pAb signal, phage display was performed to directly select mAbs with the desired specificity. Several single chain Fv (scFv)-display libraries were constructed from individual rabbits and three rounds of plate-based biopanning were performed against the GGM peptide with counter-selections against the K-ε-GG peptide (FIG. 1A). After primary screening by phage ELISA, hits were sequenced, and the unique clones were reformatted into IgGs. Four unique antibody clones (termed 1C7, 2B12, 2E9, and 2H2) were identified. The four clones had high sequence similarity but diversity occurring in multiple complementarity-determining regions (CDRs) (see FIG. 1D, showing degenerate recognition at the X position). The mAbs were characterized by ELISA against the GGM and K-ε-GG peptides, and all four clones were found to selectively bind to GGM and not K-ε-GG peptides (FIG. 1E).

While the largest pool of potential mAb targets is nascent polypeptides that predominantly start with methionine in eukaryotes, several other sources of free N-termini are present. These sources result from Met-clipping via aminopeptidases, signal peptide removal, and internal proteolysis. In the case of clipping by Met aminopeptidases (MetAP), cleavage typically occurs before Ala, Cys, Gly, pro, Ser, Thr, or Val residues (Sherman, F. et al., Bioessays 3, 27-31 (1985)). To probe whether the mAbs would also recognize tryptic peptides from these potential sites of N-terminal ubiquitination, peptides containing diglycine proceeded by each of the twenty amino acids except cysteine were evaluated. Herein, these are termed “GGX” peptides, where X represents the initial amino acid in a polypeptide sequence that contains a GG sequence addition extending from the N-terminus. Notably, mAbs 1C7 and 2H2 recognized a similar set of GGX peptides, while 2E9 and 2B12 exhibited different specificities. Collectively, these four mAbs bound 14 of the 19 GGX peptides, showing strong preference for several amino acids that would be susceptible to MetAP clipping (Sherman, F. et al., Bioessays 3, 27-31 (1985)), GGG, GGA, GGS, GGT, and GGV (FIG. 1F).

The amino acid sequences of the CDRs, heavy and light chain variable regions, and full-length heavy and light chains of 1C7, 2B12, 2E9, and 2H2, as well as consensus sequences, are provided in FIG. 1D and Tables 2A, 2B, 3, and 4, below. For the consensus sequences shown in Tables 2A, 2B, and 3, X represents any amino acid.

TABLE 2A
1C7, 2B12, 2E9, and 2H2 heavy chain variable
region CDR amino acid sequences

| Antibody | CDRH1 | CDRH2 | CDRH3 |
|---|---|---|---|
| 1C7 | SYYMN | IMFPNGKIYYATWA | DDSGDVNI |
| | (SEQ ID | (SEQ ID NO: 4) | (SEQ ID NO: 5) |
| | NO: 3) | | |
| 2B12 | RHWMN | AINESGRTYYATWA | DDDVSNF |
| | (SEQ ID | (SEQ ID NO: 12) | (SEQ ID NO: 13) |
| | NO: 11) | | |
| 2E9 | SYYMN | IMFPNGKIYYATWA | DDSGDVNI |
| | (SEQ ID | (SEQ ID NO: 20) | (SEQ ID NO: 21) |
| | NO: 19) | | |
| 2H2 | SYYMN | IMFPNGKIYYATWA | DDSGDVNI |
| | (SEQ ID | (SEQ ID NO: 28) | (SEQ ID NO: 29) |
| | NO: 27) | | |
| Consensus | XXXMN | XXXXXGXXYYATWA | DDXXXXNX |
| | (SEQ ID | (SEQ ID NO: 36) | (SEQ ID NO:37) |
| | NO: 35) | | |

TABLE 2B
1C7, 2B12, 2E9, and 2H2 light chain variable
region CDR amino acid sequences

| Antibody | CDRL1 | CDRL2 | CDRL3 |
|---|---|---|---|
| 1C7 | QSSQSVYTNNRLA | GASTLPS | LGTYDCLSADCLA |
| | (SEQ ID NO: 6) | (SEQ ID | (SEQ ID NO: 8) |
| | | NO: 7) | |
| 2B12 | QSTKSVYKYNHLS | PASTLOS | LGLYDCRSGDCNV |
| | (SEQ ID NO: 14) | (SEQ ID | (SEQ ID NO: 16) |
| | | NO: 15) | |
| 2E9 | QSSQSVYTNNRLA | GASTLPS | LGEFDCTSADCFV |
| | (SEQ ID NO: 22) | (SEQ ID | (SEQ ID NO: 24) |
| | | NO: 23) | |
| 2H2 | QSSQSVYSNNRLA | GASTLPS | LGTYDCLSADCLA |
| | (SEQ ID NO: 30) | (SEQ ID | (SEQ ID NO: 32) |
| | | NO: 31) | |
| Consensus | QSXXSVYXXNXLX | XASTLXS | LGXXDCXSXDCXX |
| | (SEQ ID NO: 38) | (SEQ ID | (SEQ ID NO: 40) |
| | | NO: 39) | |

TABLE 3
1C7, 2B12, 2E9, and 2H2 VH and VL
amino acid sequences

| | Variable | |
|---|---|---|
| Antibody | region | Amino Acid Sequence |
| 1C7 | VH | QSVKESGGRLVTPGTPLTLTCKVSGFSLSSY |
| | | YMNWVRQAPGKGLEWIGIMFPNGKIYYATWA |
| | | KGRFTISKTSTTVDLKIISPTTEDTATYFCT |
| | | GDDSGDVNIWGPGTLVTVSS |
| | | (SEQ ID NO: 1) |
| 1C7 | VL | DIVLTQTASPVSAAVGGTVTINCQSSQSVYT |
| | | NNRLAWYQQKPGQPAKEMIYGASTLPSGVSS |
| | | RFKGSGSGTQFALTISDVQCDDAATYYCLGT |
| | | YDCLSADCLAFGGGTKLEIK |
| | | (SEQ ID NO: 2) |
| 2B12 | VH | QSVEESGGGLVTPGGALTLTCTASGFSLNRH |
| | | WMNWVRQAPGKGLEWIGAINESGRTYYATWA |
| | | KGRFFISKTTTTVDLKITSPTTADTATYFCV |
| | | RDDDVSNFWGPGTLVTVSS |
| | | (SEQ ID NO: 9) |
| 2B12 | VL | DPMLTQTPSSVSAAVGGTVSINCQSTKSVYK |
| | | YNHLSWYQQKPGQPPKQLIFPASTLQSGVPS |
| | | RFSGSGSGTQFTLTISDVQCDDAATYYCLGL |
| | | YDCRSGDCNVFGGGTKLEIK |
| | | (SEQ ID NO: 10) |
| 2E9 | VH | QKQLMESGGRLVTPGTPLTLTCKVSGFSLSS |
| | | YYMNWVRQAPGKGLEWIGIMFPNGKIYYATW |
| | | AKGRFTISKTSTTVDLKIISPTTEDTATYFC |
| | | TGDDSGDVNIWGPGTLVTVSS |
| | | (SEQ ID NO: 17) |
| 2E9 | VL | DIVLTQTASPVSAAVGGTVTINCQSSQSVYT |
| | | NNRLAWYQQKPGQPAKEMIYGASTLPSGVSS |
| | | RFKGSGSGTQFALTISDVQCDDAATYYCLGE |
| | | FDCTSADCFVFGGGTEVVVK |
| | | (SEQ ID NO: 18) |
| 2H2 | VH | QSVEESRGRLVTPGTPLTLTCKVSGFSLSSY |
| | | YMNWVRQAPGKGLEWIGIMFPNGKIYYATWA |
| | | KGRFTISKTSTTVDLKIISPTTEDTATYFCT |
| | | GDDSGDVNIWGPGTLVTVSS |
| | | (SEQ ID NO: 25) |
| 2H2 | VL | DPMLTQTASPVSAAVGGTVTINCQSSQSVYS |
| | | NNRLAWYQQKPGQPPKEMIYGASTLPSGVSS |
| | | RFKGSGSGTQFTLTISDVQCDDAATYYCLGT |
| | | YDCLSADCLAFGGGTEVVVK |
| | | (SEQ ID NO: 26) |
| Consensus | VH | XXXXXESXGXLVTPGXXLTLTCXXSGFSLXX |
| | | XXMNWVRQAPGKGLEWIGXXXXXGXXYYATW |
| | | AKGRFXISKTXTTVDLKIXSPTTXDTATYFC |
| | | XXDDXXXXNXWGPGTLVTVSS |
| | | (SEQ ID NO: 33) |
| Consensus | VL | DXXLTQTXSXVSAAVGGTVXINCQSXXSVYX |
| | | XNXLXWYQQKPGQPXKXXIXXASTLXSGVXS |
| | | RFXGSGSGTQFXLTISDVQCDDAATYYCLGX |
| | | XDCXSXDCXXFGGGTXXXXK |
| | | (SEQ ID NO: 34) |

TABLE 4
1C7, 2B12, 2E9, and 2H2 light chain and heavy chain amino acid sequences

| Antibody | Heavy Chain Amino Acid Sequence | Light Chain Amino Acid Sequence |
|---|---|---|
| 1C7 | QSVKESGGRLVTPGTPLTLTCKVSGFSLSSYY | DIVLTQTASPVSAAVGG |
| | MNWVRQAPGKGLEWIGIMFPNGKIYYATWA | TVTINCQSSQSVYTNNR |
| | KGRFTISKTSTTVDLKIISPTTEDTATYFCTGD | LAWYQQKPGQPAKEMI |
| | DSGDVNIWGPGTLVTVSSASTKGPSVFPLAPC | YGASTLPSGVSSRFKGS |
| | CGDTPSSTVTLGCLVKGYLPEPVTVTWNSGT | GSGTQFALTISDVQCDD |
| | LTNGVRTFPSVRQSSGLYSLSSVVSVTSSSQP | AATYYCLGTYDCLSAD |
| | VTCNVAHPATNTKVDKTVAPSTCSKPTCPPPE | CLAFGGGTKLEIKGDPV |
| | LLGGPSVFIFPPKPKDTLMISRTPEVTCVVVDV | APTVLIFPPAADQVATG |
| | SQDDPEVQFTWYINNEQVRTARPPLREQQFN | TVTIVCVANKYFPDVT |
| | STIRVVSTLPIAHQDWLRGKEFKCKVHNKAL | VTWEVDGTTQTTGIEN |
| | PAPIEKTISKARGQPLEPKVYTMGPPREELSSR | SKTPQNSADCTYNLSST |
| | SVSLTCMINGFYPSDISVEWEKNGKAEDNYK | LTLTSTQYNSHKEYTC |
| | TTPAVLDSDGSYFLYSKLSVPTSEWQRGDVF | KVTQGTTSVVQSFNRG |
| | TCSVMHEALHNHYTQKSISRSPGK | DC |
| | (SEQ ID NO: 52) | (SEQ ID NO: 53) |
| 2B12 | QSVEESGGGLVTPGGALTLTCTASGFSLNRH | DPMLTQTPSSVSAAVG |
| | WMNWVRQAPGKGLEWIGAINESGRTYYAT | GTVSINCQSTKSVYKY |
| | WAKGRFFISKTTTTVDLKITSPTTADTATYFC | NHLSWYQQKPGQPPKQ |
| | VRDDDVSNFWGPGTLVTVSSASTKGPSVFPL | LIFPASTLQSGVPSRFSG |
| | APCCGDTPSSTVTLGCLVKGYLPEPVTVTWN | SGSGTQFTLTISDVQCD |
| | SGTLTNGVRTFPSVRQSSGLYSLSSVVSVTSSS | DAATYYCLGLYDCRSG |
| | QPVTCNVAHPATNTKVDKTVAPSTCSKPTCP | DCNVFGGGTKLEIKGD |
| | PPELLGGPSVFIFPPKPKDTLMISRTPEVTCVV | PVAPTVLIFPPAADQVA |
| | VDVSQDDPEVQFTWYINNEQVRTARPPLREQ | TGTVTIVCVANKYFPD |
| | QFNSTIRVVSTLPIAHQDWLRGKEFKCKVHN | VTVTWEVDGTTQTTGI |
| | KALPAPIEKTISKARGQPLEPKVYTMGPPREE | ENSKTPQNSADCTYNL |
| | LSSRSVSLTCMINGFYPSDISVEWEKNGKAED | SSTLTLTSTQYNSHKEY |
| | NYKTTPAVLDSDGSYFLYSKLSVPTSEWQRG | TCKVTQGTTSVVQSFN |
| | DVFTCSVMHEALHNHYTQKSISRSPGK | RGDC |
| | (SEQ ID NO: 54) | (SEQ ID NO: 55) |
| 2E9 | QKQLMESGGRLVTPGTPLTLTCKVSGFSLSSY | DIVLTQTASPVSAAVGG |
| | YMNWVRQAPGKGLEWIGIMFPNGKIYYATW | TVTINCQSSQSVYTNNR |
| | AKGRFTISKTSTTVDLKIISPTTEDTATYFCTG | LAWYQQKPGQPAKEMI |
| | DDSGDVNIWGPGTLVTVSSASTKGPSVFPLAP | YGASTLPSGVSSRFKGS |
| | CCGDTPSSTVTLGCLVKGYLPEPVTVTWNSG | GSGTQFALTISDVQCDD |
| | TLTNGVRTFPSVRQSSGLYSLSSVVSVTSSSQP | AATYYCLGEFDCTSAD |
| | VTCNVAHPATNTKVDKTVAPSTCSKPTCPPPE | CFVFGGGTEVVVKGDP |
| | LLGGPSVFIFPPKPKDTLMISRTPEVTCVVVDV | VAPTVLIFPPAADQVAT |
| | SQDDPEVQFTWYINNEQVRTARPPLREQQFN | GTVTIVCVANKYFPDV |
| | STIRVVSTLPIAHQDWLRGKEFKCKVHNKAL | TVTWEVDGTTQTTGIE |
| | PAPIEKTISKARGQPLEPKVYTMGPPREELSSR | NSKTPQNSADCTYNLSS |
| | SVSLTCMINGFYPSDISVEWEKNGKAEDNYK | TLTLTSTQYNSHKEYTC |
| | TTPAVLDSDGSYFLYSKLSVPTSEWQRGDVF | KVTQGTTSVVQSFNRG |
| | TCSVMHEALHNHYTQKSISRSPGK | DC |
| | (SEQ ID NO: 56) | (SEQ ID NO: 57) |
| 2H2 | QSVEESRGRLVTPGTPLTLTCKVSGFSLSSYY | DPMLTQTASPVSAAVG |
| | MNWVRQAPGKGLEWIGIMFPNGKIYYATWA | GTVTINCQSSQSVYSNN |
| | KGRFTISKTSTTVDLKIISPTTEDTATYFCTGD | RLAWYQQKPGQPPKE |
| | DSGDVNIWGPGTLVTVSSASTKGPSVFPLAPC | MIYGASTLPSGVSSRFK |
| | CGDTPSSTVTLGCLVKGYLPEPVTVTWNSGT | GSGSGTQFTLTISDVQC |
| | LTNGVRTFPSVRQSSGLYSLSSVVSVTSSSQP | DDAATYYCLGTYDCLS |
| | VTCNVAHPATNTKVDKTVAPSTCSKPTCPPPE | ADCLAFGGGTEVVVKG |
| | LLGGPSVFIFPPKPKDTLMISRTPEVTCVVVDV | DPVAPTVLIFPPAADQV |
| | SQDDPEVQFTWYINNEQVRTARPPLREQQFN | ATGTVTIVCVANKYFP |
| | STIRVVSTLPIAHQDWLRGKEFKCKVHNKAL | DVTVTWEVDGTTQTTG |
| | PAPIEKTISKARGQPLEPKVYTMGPPREELSSR | IENSKTPQNSADCTYNL |
| | SVSLTCMINGFYPSDISVEWEKNGKAEDNYK | SSTLTLTSTQYNSHKEY |
| | TTPAVLDSDGSYFLYSKLSVPTSEWQRGDVF | TCKVTQGTTSVVQSFN |
| | TCSVMHEALHNHYTQKSISRSPGK | RGDC |
| | (SEQ ID NO: 58) | (SEQ ID NO: 59) |

Overall, these data revealed that four novel anti-GGX mAbs were generated that selectively recognized tryptic diglycine-containing linear peptides with broad specificity at the third position (GGX), and lacked cross-reactivity to isopeptide-linked diglycine-modified lysine containing peptides that correspond to canonical ubiquitination sites.

### Example 2: Structural Basis for GGX Peptide Recognition

The following example describes the determination of an x-ray crystal structure of the 1C7 anti-GGX Fab bound to a GGM peptide.

### Materials and Methods

### Crystallization Conditions and Structure Determination

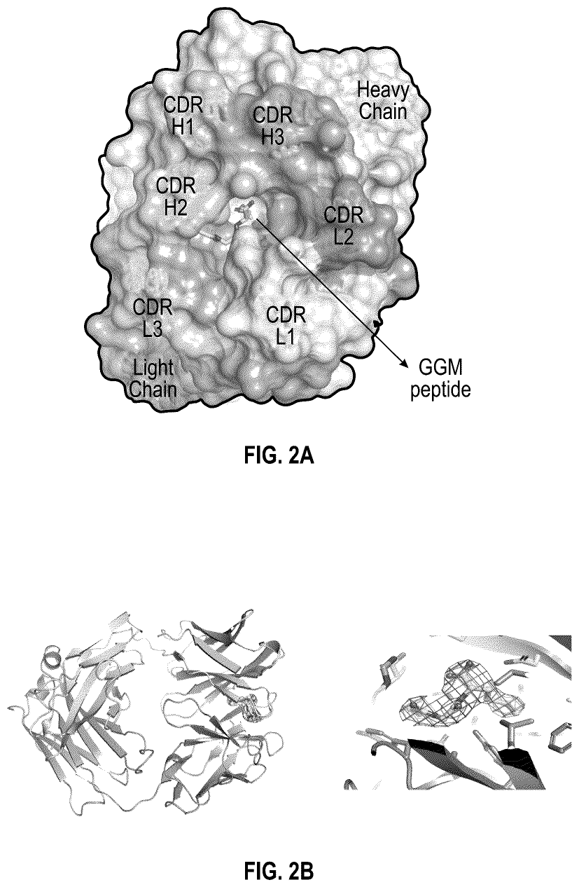

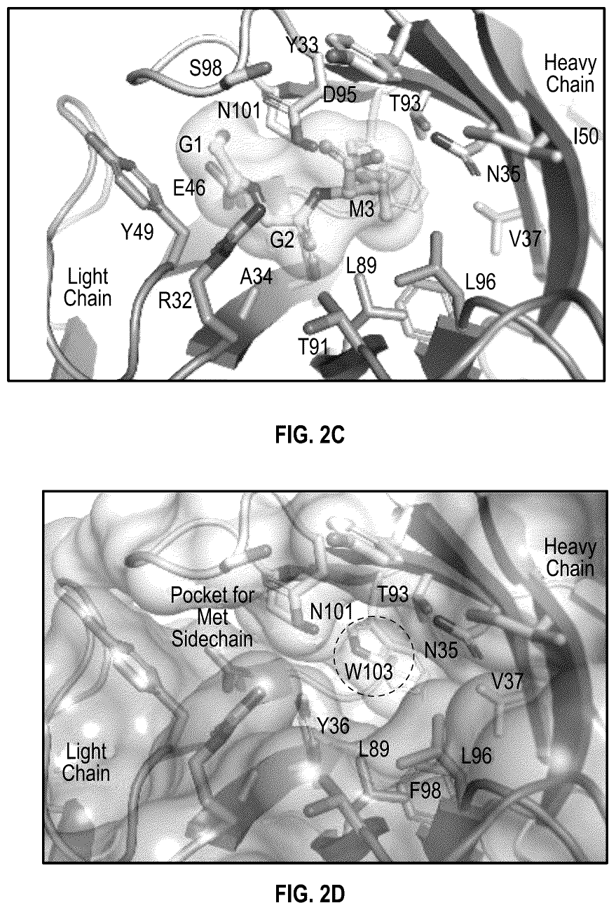

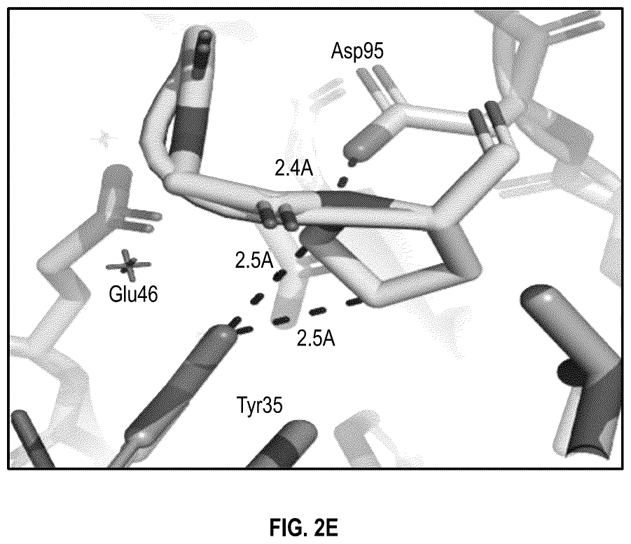

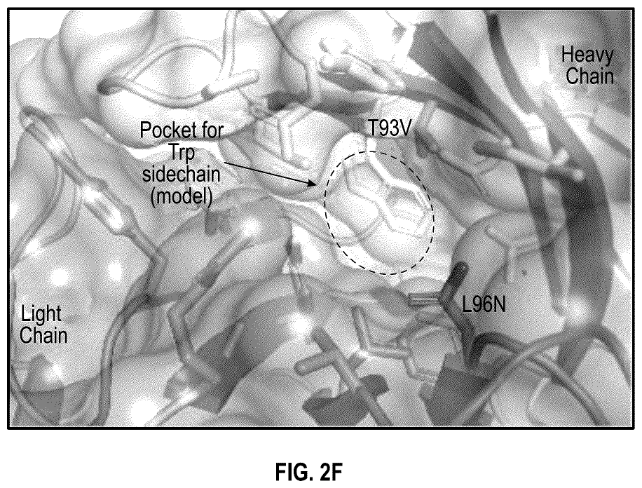

The 1C7 Fab-GGM complex was screened for crystallization using the hanging drop method with 1:1 ratio of protein:well-solution. Crystals were observed in multiple conditions, with the best condition being 2M ammonium sulfate and 0.1 M TRIS pH 7.5. Upon optimization, single crystals grew to −200 mm in 2M ammonium sulfate and 0.1M MES pH 6.5. The crystals matured over 2 weeks and were flash frozen with 20% (v/v) ethylene glycol in 2 M ammonium sulfate and 0.1 M MES pH 6.5. The diffraction data was collected at Advanced Light Source (ALS) beamline 5.0.2 at a temperature of 100 K. The data were processed to 2.85 Å resolution with HKL2000 (Otwinowski, Z. & Minor, W. Methods in Enzymology 276, (1997)), and the phases were obtained with PHENIX by molecular replacement with a model rabbit Fab (PDB: 4ZTP). The structure was built using COOT (Emsley, P. & Cowtan, K. Acta Crystallogr Sect D Biological Crystallogr 60, 2126-2132 (2004)), and refined using PHENIX (Adams, P. D. et al., Acta Crystallogr Sect D Biological Crystallogr 66, 213-221 (2010)). The final model was generated after addition of GGM peptide, water molecules and buffer molecules (see FIGS. 2A-2E, and Table 5).

### Results

To gain insight into the selectivity of the anti-GGX mAbs for linear diglycine-containing peptides, the x-ray crystal structure of the 1C7 Fab bound to a GGM peptide was determined at 2.85 Å resolution. Table 5, below, provides data collection and refinement statistics for the 1C7 Fab GGM peptide co-crystal structure, with values in parentheses for the highest-resolution shell.

TABLE 5
Data collection and refinement statistics for
the 1C7 Fab GGM peptide co-crystal structure.
Data collection

| Space group | P 4₃ 2₁ 2 | |
|---|---|---|
| Cell dimensions | | |
| a, b, c (Å) | 163.80, 163.80, 127.47 | |
| α, β, γ | 90, 90, 90 | |
| Resolution (Å) | 48.08-2.85 | (2.92-2.85) |
| Rₚᵢₘ | 0.039 | (0.477) |
| I/σ (I) | 22.40 | (1.46) |
| Completeness (%) | 99.5 | (98.7) |
| Redundancy | 6.6 | (6.2) |
| CC_{1/2} | 0.999 | (0.806) |
| CC* | 0.998 | (0.945) |

Refinement

| Resolution (Å) | 2.85 | |
|---|---|---|
| No. of reflections | 40874 | (2701) |

| | |
|---|---|
| R_{work}/R_{free} | 211/260 |
| No. of atoms | 6568 |
| Protein | 6479 |
| Ligand/ion | 50 |
| Water | 39 |
| Average B-factors | 78.0 |
| Protein | 78.20 |
| Ligand/ion | 106.3 |
| Water | 73.1 |
| R.m.s deviations | |
| Bond lengths (Å) | 0.009 |
| Bond angles (°) | 1.114 |

There were two Fab-GGM complexes in the asymmetric unit, with well-defined electron density for the GGM peptide that binds in a pocket at the interface of the heavy chain (HC) and light chain (LC) CDRs (FIG. 2A, FIG. 2B). The interaction of GGM peptide and Fab has a buried surface area of 247.5 Å². Interestingly, this pocket at the LC-HC interface is commonly used by antibodies to recognize haptens (Finlay, W. J. J. & Almagro, J. C. Front Immunol 3, 342 (2012)). Close inspection of the Fab-peptide complex revealed a series of hydrogen bonds that facilitate recognition of the diglycine portion of the peptide. The sidechains of HC Asp95 and LC Glu46 make five hydrogen bonds with the backbone of the diglycine, including with the amino terminus and the two amides (FIG. 2C). The negative charge of the two carboxylates appeared to neutralize the positive charge of the amino terminus, which was surrounded by the Fab residues and excluded from the solvent. In addition, tight packing of diglycine against LC Ala34, LC Tyr36, and LC Tyr49 likely sterically blocked recognition of a non-Gly residue at either of the first two positions in the peptide. This binding mode was further stabilized by a hydrogen bond between the HC Asp95 sidechain and backbone amine of Met (FIG. 2C).

Inspection of the Met-binding pocket revealed the structural basis for the desired degenerate amino acid specificity at this position. This pocket was lined with HC residues Asn35, Val37, Thr93, Asn101, and Trp103 on one side, and LC residues Tyr36, Leu89, Leu96, and Phe98 on the other side (FIG. 2D). The closest contact made with the methionine sidechain is a 3.1 Å hydrogen bond between the sulfur atom and HC Thr93 (FIG. 2C). This analysis indicated a loosely-packed pocket with both hydrophobic and hydrophilic character, which, without wishing to be bound by theory, is thought to enable the recognition of a broad set of amino acids. Lack of recognition of Trp, Lys, Tyr, and Arg was readily explained by steric clashes with multiple side chains that line this pocket. In the case of pro, multiple clashes between the HC Asp95 and LC Tyr35 side chains in the antibody and the pro side chain in the peptide would occur (FIG. 2E).

A key feature of this antibody was the lack of recognition of the highly similar K-ε-GG peptides. Therefore, how a K-ε-GG peptide would interact with the Fab was analyzed, assuming that the mode of GG recognition is the same as for the GGM peptide. It was hypothesized that the lysine side chain could follow a similar trajectory as the main chain of GGM, however, branching at the Lys Ca position (i.e., residues before and after Lys in the peptide) would sterically clash with CDRH3 and HC Tyr33 preventing binding to the mAb.

Since both 2E9 and 2B12 mAbs had sequence similarity to 1C7 but exhibited altered recognition profiles, the potential structural basis for this result was examined. Within the GGM-binding pocket, 2E9 had two differences (LC Thr91Glu and Leu96Phe) which would compact the Met pocket, and, without wishing to be bound by theory, likely preventing recognition of a broad set of residues (FIG. 2D). The six residue differences in 2B12 would even more dramatically reshape the Met pocket. For example, the LC Thr91Leu and HC Thr93Val increased the hydrophobicity of the pocket which may explain the unique ability to bind GGW compared to the other mAbs (FIG. 2D). A model of a pocket in 2B12 which may bind a Trp sidechain is provided in FIG. 2F, with the HC Thr93Val and LC Leu96Asn residues indicated.

Collectively, the structural studies described herein elucidated how these antibodies achieve degenerate recognition of GGX while avoiding recognition of the highly similar K-ε-GG.

### Example 3: Anti-GGX mAbs Selectively Enrich GGX Peptides from Cell Lysates

The following example describes experiments investigating immunoaffinity enrichment by the anti-GGX monoclonal antibodies of peptides from complex cell lysates. Specifically, immunoaffinity enrichment followed by mass spectrometry was performed to identify proteins bound by the anti-GGX antibodies in HEK293 cell lysates.

### Materials and Methods

### Pilot Mass Spectrometry Experiments Demonstrating Reagent Selectivity

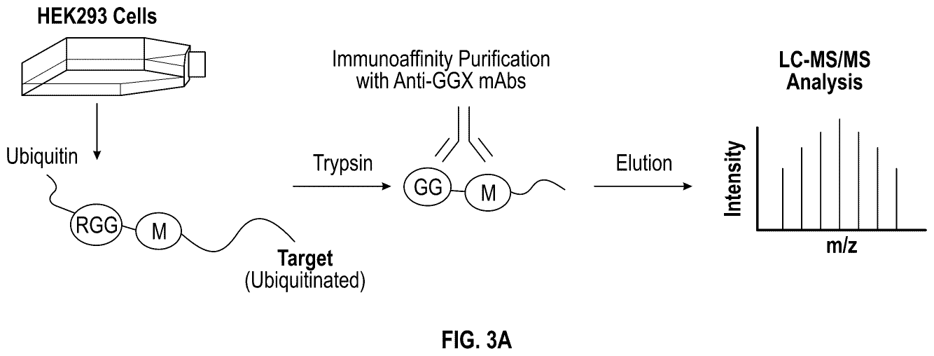

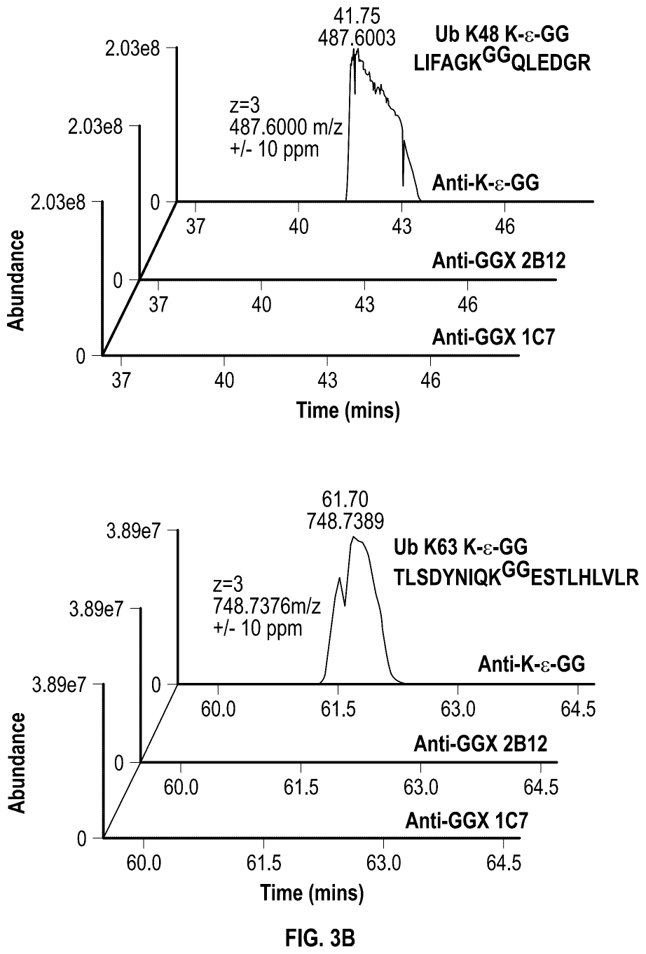

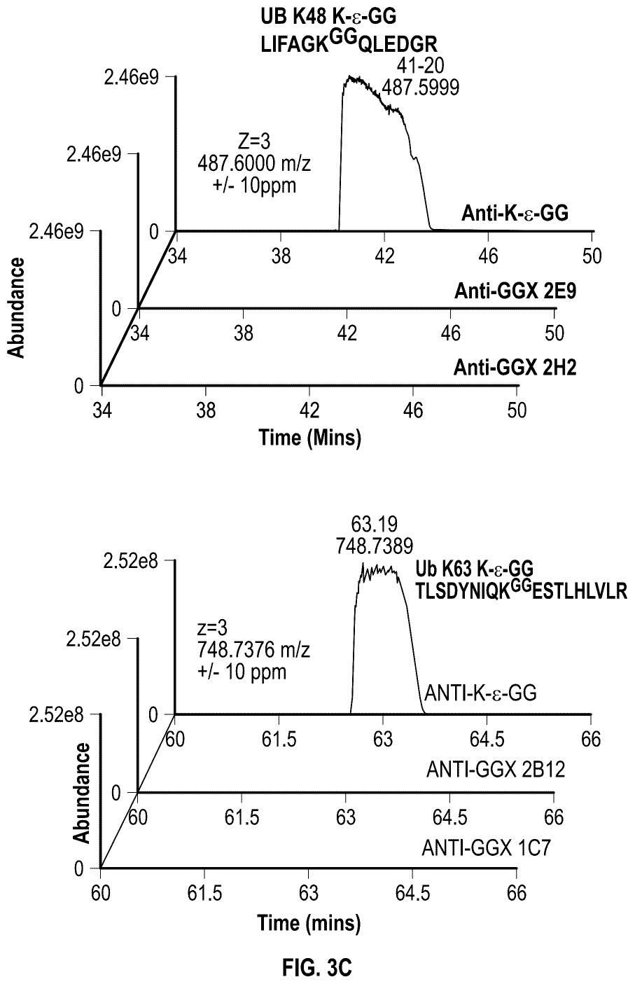

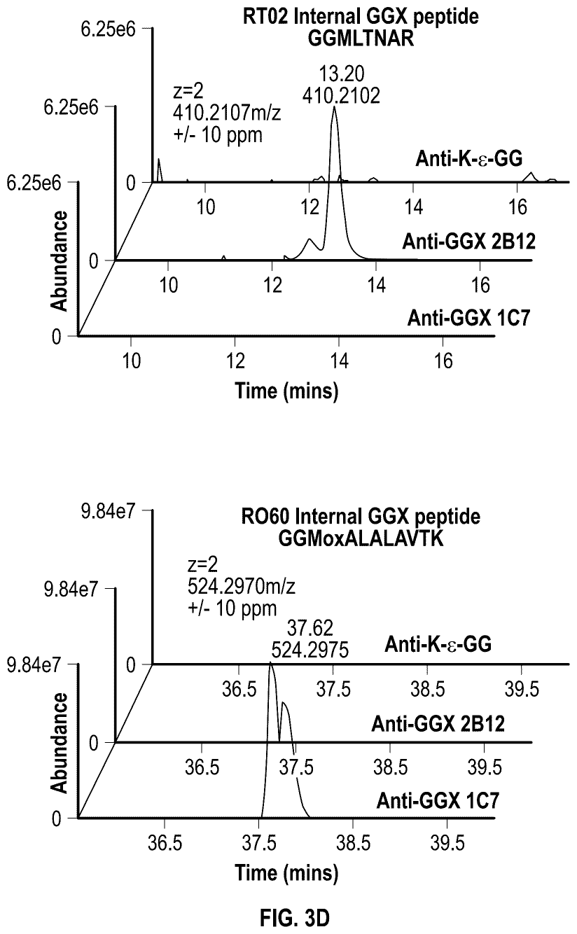

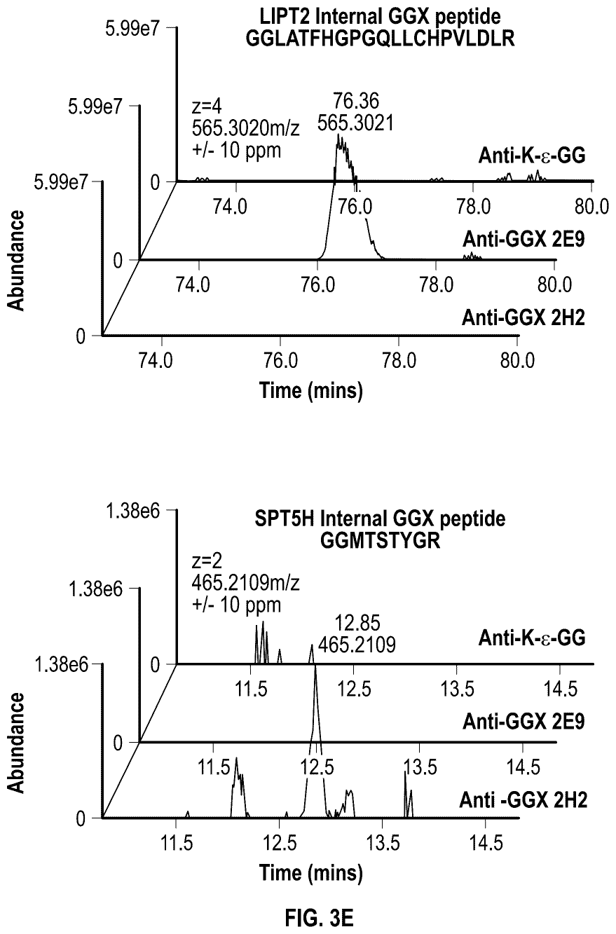

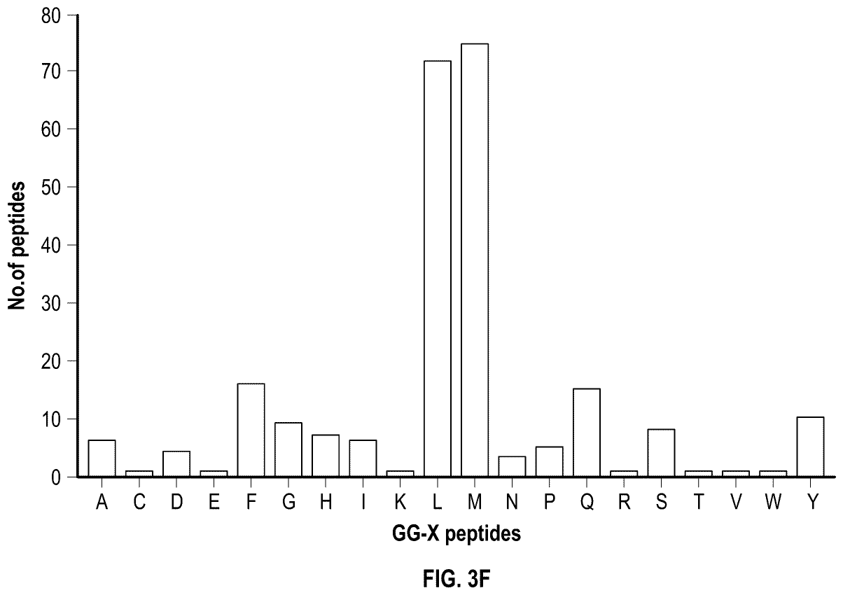

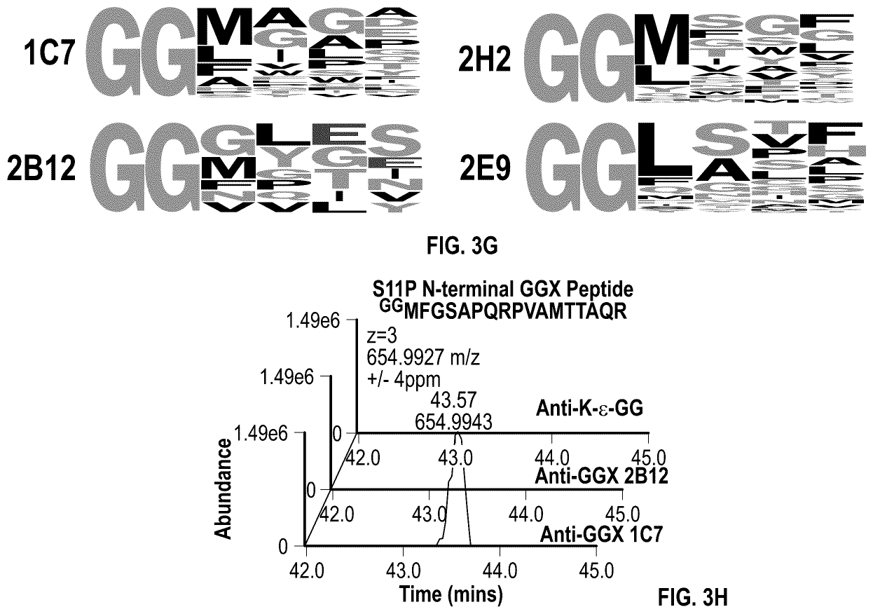

40 mgs of protein lysate was prepared from confluent HEK293 T cells and digested with Trypsin (promega). Tryptic peptides in PTMScan® IAP buffer (Cell Signaling Technologies) were incubated with 80 μg of anti-GGX or anti-K-ε-GG mAb (Cell Signaling Technology®) for 30 minutes at 4° C. (see FIG. 3A). Subsequently, 80 μL of protein G agarose slurry was added to the antibody-peptide mixture for an additional 30 minutes at 4° C. For the MS experiment in which the four GGX mAbs were pooled for use in peptide immunoaffinity enrichment, 50 μg of each mAb was mixed together before being put into contact with trypsin digested and desalted peptides.

### Mass Spectrometry

Resuspended samples were analyzed by LC-MS/MS in a 120 minute total run time method. Peptides were separated on a nanoAcquity UPLC (Waters) and introduced to either an Orbitrap Elite™ or Q Exactive™ HF mass spectrometer (ThermoFisher) by electrospray ionization. Thirty to forty percent of each sample was loaded onto a 100 m×100 mm Waters 1.7-μm BEH-130 C18 column and separated by low pH reversed phase chromatography (solvent A: 0.1% FA/98% water/2% ACN, solvent B: 0.1% FA/98% ACN/2% water) at a flowrate of 1 l/minute using a two-stage linear gradient applied over 90 minutes. In the first stage, solvent B increased from 2% to 25% over a span of 85 minutes, followed by the second stage with solvent B increasing from 25% to 40% over a span of 5 minutes. Both the Orbitrap Elite™ and Q Exactive™ HF mass spectrometers were operated in data dependent mode with the top 15 and top 10 most abundant ions selected for MS2 fragmentation, respectively. Mass spectrometer specific settings used for analysis, optimized for each instrument, were as follows.

Orbitrap Elite™ Fourier transform mass spectrometry (FTMS1) scans were collected at 60,000 resolution, an automatic gain control (AGC) target of 1×10⁶, and a maximum injection time of 200 ms. Ion trap mass spectrometry (ITMS2) was performed using collision-induced dissociation (CID) set at 35% normalized collision energy, an automatic gain control (AGC) target of 1×10³, and a maximum injection time of 100 ms.

Q Exactive™ HF FTMS1 scans were collected at 60,000 resolution, an AGC target of 3×10⁶, and a maximum injection time of 60 ms. FTMS2 was performed using higher-energy collisional dissociation (HCD) set at 30% normalized collision energy, collected at 15,000 resolution, an AGC target of 1×10⁵, and a maximum injection time of 75 ms.

### Data Analysis

Datafiles were searched using Mascot (Matrix Science) against a target-decoy database containing Uniprot human (downloaded August, 2017) and common contaminant sequences. A precursor ion mass tolerance of 25 ppm, fragment ion tolerance of 0.8 Da (ITMS2) or 0.02 Da (FTMS2), and semi-tryptic enzyme specificity were employed. Carbamidomethylated cysteine (+57.0215 Da) was set as a fixed modification and methionine oxidation (+15.9949 Da), K-ε-GG (+114.0429), and N-terminal GG (+114.0429) were considered as variable modifications. Peptide spectral matches were filtered at the peptide level using linear discriminate analysis to a false discovery rate of 5 percent with subsequent filtering based on sequence features relevant to the biology being interrogated.

### Results

Lysates from unstimulated HEK293 cells were digested with trypsin, and immunoaffinity enrichment was performed for GGX peptides using each of the four anti-GGX mAbs individually. The resulting peptide pools were analyzed by liquid chromatography-tandem mass spectrometry (LC-MS/MS) (FIG. 3A). In parallel, immunoaffinity enrichment was performed with the anti-K-ε-GG mAb as a control. Given the high abundance of K48 and K63-linked Ub chains present in these lysates, these target peptides were used to confirm the selectivity of the novel mAbs for immunoaffinity enrichment of GGX peptides over abundant K-ε-GG peptides. Extracted ion chromatograms (XIC) for representative peptide ions corresponding to K48 and K63-linked Ub chains were prepared from LC-MS data for the anti-GGX and anti-K-ε-GG enriched samples to compare their levels. Compared to the anti-K-ε-GG mAb, which showed strong enrichment of isopeptide linked K48 and K63 Ub peptides, no signal was detected when any of the four anti-GGX mAbs was used for enrichment (FIG. 3B, FIG. 3C).

Next, peptide sequences that were enriched by the anti-GGX mAbs were investigated. Given the frequency of glycine, lysine and arginine residues in the proteome, many GGX peptides are encoded in the proteome, stemming from proteins that contain naturally occurring internal GGX sequence motifs preceded by a trypsin cleavage site (R/KGGXXXX). As predicted, many such peptides were detected in this experiment. Notably, extracted ion chromatograms for representative internal GGX sequences applied across each enriched sample demonstrated specific signal in anti-GGX mAb enriched samples, but none following immunoaffinity enrichment with the anti-K-ε-GG mAb. Combined with the ELISA data, these results demonstrated the selectivity of the anti-GGX mAbs for the sequences being targeted (FIG. 3D, FIG. 3E).

Just as with N-terminally ubiquitinated proteins, internal GGX peptides only become exposed after trypsin cleavage and provide valuable insights into the sequence preferences of each mAb. Using these internal peptides, the amino acid preference at the third position was determined. Consistent with the panning strategy, ELISAs, and structural data, a strong preference for methionine and leucine at the third position was observed, with the next most prevalent amino acids being phenylalanine and glutamine (FIG. 3F). To further profile sequence specificity, sequence logos were generated for each anti-GGX mAb (FIG. 3G) (Schneider, T. D. & Stephens, R. M. Nucleic Acids Res 18, 6097-6100 (1990)). Again, the sequence logos show overarching preference for methionine and leucine at the third position, but importantly, a diversity of other amino acids at positions 3-6 (FIG. 3G). These data reconfirmed the ELISA results showing that each mAb enriched for a unique set of peptides with partial overlap between individual mAbs, especially when considering the differences in positions 4-6 (Table 6). Based on these data, a PTMscan® protocol was established using an equimolar mixture of the four anti-GGX mAbs for subsequent MS experiments to ensure the broadest coverage of potential peptides (see Example 4).

TABLE 6
Unique and shared internal GGX sequences from anti-GGX mAbs

| Antibody | Unique sequences | Shared sequences |
|---|---|---|
| 1C7 | 26 | 16 |
| 2E9 | 41 | 14 |
| 2H2 | 47 | 20 |
| 2B12 | 8 | 8 |

Focusing on sites of N-terminal ubiquitination, the data was manually inspected and peptide spectral matches (PSMs) were filtered for those bearing a diglycine remnant at the initiator methionine, or neo-N-terminus. Peptides bearing a 114.0429 Da mass addition, corresponding to the mass of diglycine, were identified, and then the genome encoded polypeptide sequence was confirmed to not contain a diglycine sequence immediately preceded by a trypsin sensitive R/K residue. Following rigorous filtering, more than a half dozen proteins with putative N-terminal ubiquitination sites were identified (Table 7). One example was a putative N-terminal ubiquitination site observed on Serine/threonine-protein kinase 11-interacting protein (STK11IP) (FIG. 3H, FIG. 3I), in addition to several sites that have been previously described (Akimov, V. et al., Nat Struct Mol Biol 25, 631-640 (2018)).

TABLE 7
Proteins with putative N-terminal ubiquitination sites identified
in pilot immunoaffinity enrichment and MS experiment

| | UniProt Protein |
|---|---|
| UniProt Entry Name | Accession No. |
| DCTP1_HUMAN | Q9H773 |
| F13A_HUMAN | P00488 |
| HNRPK_HUMAN | P61978 |
| PUR9_HUMAN | P31939 |
| RFA1_HUMAN | P27694 |
| RPB7_HUMAN | P62487 |
| S11IP_HUMAN | Q8N1F8 |
| UCHL5_HUMAN | Q9Y5K5 |

Overall, this study demonstrated the selective ability of these anti-GGX mAbs to enrich a broad panel of both internal, genome-encoded GGX peptide sequences exposed by trypsin digestion, and GGX peptides stemming from N-terminal ubiquitination. This pilot MS experiment validated the utility of the anti-GGX mAbs, but yielded fewer than a dozen putative N-terminally ubiquitinated substrates from endogenous HEK293 cells. This result was consistent with existing literature, confirming the low basal levels of the N-terminal ubiquitin modification (Akimov, V. et al., Nat Struct Mol Biol 25, 631-640 (2018)).

### Example 4: Proteomic Identification of Putative UBE2W Substrates

The following example describes the generation of a HEK293 cell line with doxycycline (Dox)-inducible expression of UBE2W, the gene encoding ubiquitin-conjugating enzyme E2. Further, the Dox-inducible UBE2W HEK293 cell line was used to identify peptides bound by the anti-GGX mAbs in an immunoaffinity enrichment MS experiment.

### Materials and Methods

Immunoaffinity Enrichment of GGX and K-ε-GG Peptides from UBE2W Expressing Cells for Label Free Quantitative (LFQ) Analysis by Mass Spectrometry

HEK293 cells inducibly expressing ubiquitin-conjugating enzyme E2 (UBE2W) and matched controls (i.e., cells expressing the E3 ubiquitin-protein ligase RNF4 or both UBE2W and RNF4) were lysed under fully denaturing conditions (8M urea, 20 mM HEPES pH 8.0, 1 mM sodium orthovanadate, 2.5 mM sodium pyrophosphate, 1 mM β-glycerophosphate). Lysates were microtip sonicated on ice (2×30 sec), and cleared by high-speed ultracentrifugation (18,000×g, 15 min). 40 mg of each lysate was taken forward for reduction (4.1 mM dithiothreitol, 60 minutes at 37° C.), alkylation (9.1 mM iodoacetamide, 15 minutes at room temperature), 4-fold dilution, and then subjected to overnight digestion with a combination of lysyl-endopeptidase (Wako) and sequencing grade trypsin (promega) both at an enzyme to protein ratio of 1:100, the latter which was added 4 hours following incubation with the former. Digested peptides were acidified with TFA to a final concentration of 1%, cleared by centrifugation (18,000×g, 15 min), desalted by Sep-Pak® C18 gravity flow solid-phase extraction (Waters), and lyophilized for 48 hours. Dry peptides were reconstituted in 1 mL 1×IAP buffer (Cell Signaling Technology) and clarified via high-speed centrifugation (18,000×g, 10 min) for subsequent immunoaffinity enrichment.

Peptides were subjected to two serial rounds of immunoaffinity enrichment, both performed at 4° C. on a PhyNexus MEA2 automated purification system using 1 mL Phytips (Phynexus) packed with 20 μL ProPlus resin coupled to either 200 μg of anti-GGX antibody cocktail (i.e., an equimolar mix of 1C7, 2B12, 2E9, and 2H2) or 200 μg of anti-K-ε-GG (Cell Signaling Technology) antibody. Enrichment was performed in the following order: anti-GGX IP for peptides containing a diglycine modified N-terminus (GGX), and anti-K-ε-GG IP for peptides containing diglycine modified lysine residues (K-ε-GG).

Immunoaffinity enrichment on the PhyNexus MEA2 was performed as previously described (Phu, L. et al., Mol Cell 77, 1107-1123.e10 (2020)). Briefly, Phytip columns were equilibrated for 2 cycles (1 cycle=aspiration and dispensing, 0.9 mL, 0.5 mL/min) with 1 mL 1×IAP buffer prior to contact with peptides, incubated with peptides for 16 cycles of capture, and washed for 6 cycles (2× with 1 mL 1×IAP buffer followed by 4× with 1 mL water). Captured peptides were eluted with 60 μL 0.15% TFA in 8 cycles where the volume aspirated/dispensed was adjusted to 0.06 mL. Eluted peptides were subsequently desalted using C18 stage-tips (Rappsilber, J. et al., Nat Protoc 2, 1896-1906 (2007)) and Speed-Vac (ThermoFisher) dried to completion.

Enriched GGX peptides were reconstituted in 2% acetonitrile (ACN)/0.1% formic acid (FA) and analyzed in duplicate (40% each injection) by LC-MS/MS on an Orbitrap Fusion™ Lumos™ mass spectrometer (ThermoFisher) coupled to a Dionex UltiMate 3000 RSLC (ThermoFisher) employing a 100 μm×250 mm PicoFrit (New Objective) column packed with 1.7-μm BEH-130 C18 resin (Waters). Low pH reversed-phase separation (solvent A: 0.1% FA/98% water/2% ACN, solvent B: 0.1% FA/98% ACN/2% water) was performed at 450 nL/minute on a 96 minute two step linear gradient where solvent B increased from 2% to 35% over 102 minutes and then from 35% to 50% over 2 minutes with a total run time of 120 minutes. The Orbitrap Fusion™ Lumos™ was operated in data dependent mode whereby FTMS1 scans were collected at 240,000 resolution with an AGC target of 1×10⁶ and a maximum injection time of 50 ms. MS2 scans on the top 15 most intense precursors with charge states of 2-4 were collected in the ion trap with HCD fragmentation at a normalized collision energy of 30%, an AGC target of 2.0×10⁴, and a max injection time of 11 ms.

For the duplicate injections, MS2 spectra were analyzed in the Orbitrap rather than the ion trap. OTMS2 AGC target was set to 2.0×10⁵ with a max injection time of 54 ms.

Immunoaffinity Enrichment of GGX and K-ε-GG Peptides from UBE2W and/or RNF4 Expressing Cells for LC-MS Analysis

Immunoaffinity enrichment of GGX and K-ε-GG peptides from 40 mg each of HEK293 cells either non-induced (N=3) or inducibly expressing the E3 ubiquitin-protein ligase RNF4 (N=2), UBE2W (N=3), or the combination (N=3) was performed as detailed above with the following modifications.

Peptides were subjected to three serial rounds of immunoaffinity enrichment, all performed as described above on an MEA2 automated purification system (Phynexus) with either 200 μg of anti-GGX antibody cocktail or 200 μg of anti-K-ε-GG (Cell Signaling Technology) antibody. Enrichment was performed in the following order: anti-GGX IP for peptides containing a diglycine modified N-terminus (GGX), anti-K-ε-GG IP for peptides containing diglycine modified lysine residues (K-ε-GG), followed by anti-GGX IP for GGX once more.

The enriched peptides from first (GGX) and second (K-ε-GG) round immunoprecipitations were subsequently prepared for tandem mass tagging (TMT-11) multiplexed quantitative analysis as previously described (Rose, C. M. et al., Cell Syst 3, 395-403.e4 (2016); Phu, L. et al., Mol Cell 77, 1107-1123.e10 (2020)) while the enriched GGX peptides from the third round were prepared for label free quantitative mass spectrometric analysis.

### TMT-11 Multiplexed Sample Prep

Eluates containing enriched GGX or K-ε-GG peptides were desalted using C18 stage-tips, SpeedVac dried to completion, and reconstituted in 25 μL 200 mM HEPES pH 8.0 for subsequent isobaric labeling with 11-plex tandem mass tagging (TMT) reagents (ThermoFisher). Each vial of TMT reagent was allowed to thaw for 5 minutes at room temperature, spun down using a benchtop centrifuge, and resuspended in 41 μL of anhydrous acetonitrile (ACN). To each eluate, 8 μL of TMT reagent was added along with 2 μL of ACN to reach an optimal labeling reaction final ACN concentration of 29%. After 1 hour incubation at room temperature, the reaction was quenched by addition of 4 μL of 5% hydroxylamine for 15 minutes. Labeled peptides were combined and dried by vacuum centrifugation.

The TMT labeled GGX peptides were resuspended in solvent A (2% acetonitrile (ACN)/0.1% formic acid (FA) and split into two portions, 40% and 60%, the former slated for LC-MS/MS analysis without further manipulation and the latter subjected to additional offline high pH reversed-phase fractionation using an RPS cartridge on the AssayMap (Agilent) employing a 0.1% triethylamine/acetonitrile based elution buffer. Six fractions were collected (F1: 12% ACN, F2: 17%% ACN, F3: 22% ACN, F4: 27% ACN, F5: 32% ACN, F6: 80% ACN). Fractionated GGX peptides were subsequently lyophilized and resuspended in solvent A for LC-MS/MS analysis.

For TMT labeled K-ε-GG peptides, high pH reversed-phase fractionation was performed using a commercially available kit (ThermoFisher). Upon resolubilization in 0.15% TFA, fractionation was performed according to the manufacturer's protocol with a modified elution scheme where 11 fractions were collected (F1: 13.5% ACN, F2: 15% ACN, F3: 16.25% ACN, F4: 17.5 ACN, F5: 20% ACN, F6: 21.5% ACN, F7: 22.5% ACN, F8: 23.75% ACN, F9: 25% ACN, F10: 27.5% ACN and F11: 30% ACN) and then combined into 6 fractions (F1+F6, F2+F7, F8, F3+F9, F4+F10, F5+F11). Peptides were lyophilized and resuspended in 10 μL solvent A for LC-MS/MS analysis.

LC-MS/MS analysis on the unfractionated GGX sample was performed on an Fusion™ Lumos™ mass spectrometer (ThermoFisher) coupled to a NanoAcquity® UPLC (Waters) system equipped with a 100 μm×250 mm PicoFrit® column (New Objective) packed with 1.7 uM BEH-130 C18 (Waters). Low pH reversed-phase separation (solvent A: 0.1% FA/98% water/2% ACN, solvent B: 0.1% FA/98% ACN/2% water) was performed at 500 nL/minutes on a 163 minute two step linear gradient where solvent B was ramped from 2% to 30% over 158 minutes and then from 30% to 75% over 5 minutes with a total run time of 180 minutes. The Fusion™ Lumos™ collected FTMS1 scans at 120,000 resolution with an AGC target of 1×10⁶ and a maximum injection time of 50 ms. FTMS2 scans on precursors with charge states of 2-6 were collected at 15,000 resolution with CID fragmentation at a normalized collision energy of 35%, an AGC target of 5.0×10⁴, and a max injection time of 200 ms. Synchronous-precursor-selection (SPS) MS3 scans were analyzed in the Orbitrap at 50,000 resolution with the top 8 most intense ions in the MS2 spectrum subjected to HCD fragmentation at a normalized collision energy of 55%, an AGC target of 1.5×10⁵, and a max injection time of 400 ms.

LC-MS/MS analysis on the fractionated GGX peptides was performed as described above with the following exceptions. Liquid chromatography was performed using a Dionex Ultimate 3000 RSLC (ThermoFisher) on an Aurora Series 25 cm×75 μm I.D. column (IonOpticks) running at a reduced flowrate of 300 nL/minute and a modified gradient whereby solvent B ramped from 2% to 30% over 135 minutes and 30% to 50% over 15 minutes.

LC-MS/MS analysis on the fractionated K-ε-GG peptides was performed exactly as described for the unfractionated GGX sample with a modification to the MS method restricting precursor ions selected for fragmentation to those with charge states 3-6.

In the TMT analysis, a series of large and unanticipated features were observed in the MS1 data that appeared to affect the performance of the data dependent method. These features included a series of intense peaks eluting across the chromatogram which were identified as the compounded signal of abundant, internal GGX peptides present in each of the 11 samples. It was hypothesized that this signal overshadowed lower intensity signals from N-terminal ubiquitinated GGX peptides of interest that are expected to be present in only a subset (UBE2W only, Combo) of the 11 samples. In an effort to minimize competition of high abundance internal GGX peptides for signal and recover additional identifications that may have been obscured, immunoaffinity enrichment using the flow-through peptides from the TMT labeling experiment and subjected those enriched peptides to LC-MS for label free quantitative (LFQ) analysis, as above. For TMT multiplexing data, raw MS data were searched using Mascot against a Uniprot human target-decoy database (downloaded August, 2017) containing common contaminant sequences with a ppm precursor ion mass tolerance of 25 ppm, fragment ion tolerance of 0.02 Da, and semi-tryptic enzyme specificity. Carbamidomethylated cysteine residues (+57.0215 Da) and TMT labeled n-terminus (+229.1629) were set as a fixed modifications with methionine oxidation (+15.9949 Da), K-ε-GG (+114.0429), and N-terminal GG (+114.0429) considered as variable modifications. Peptide spectral matches for each run were filtered using LDA to a FDR of 3% and a ppm mass tolerance between −5 and 4 from the theoretical precursor m/z. TMT-MS3 quantification was performed using Mojave (Zhuang, G. et al., Sci Signal 6, ra25-ra25 (2013)). Quantification and statistical testing of the TMT proteomics data was performed using MSstatsTMT v1.6.3, an open-source R/Bioconductor package (Huang, T. et al., Mol Cell Proteomics 19, mcp.RA120.002105 (2020)). Prior to MSstatsTMT analysis, PSMs were filtered from further analysis if they were (1) from decoy proteins; (2) from peptides with length less than 7; (3) with isolation specificity less than 50%; (4) with reporter ion intensity less than 256; or (5) with summed reporter ion intensity (across all eleven channels) lower than 30,000. Redundant PSMs (i.e., multiple PSMs in one MS run that map to the same peptide) were summarized by first taking the maximum reporter ion intensities per peptide and channel and then selecting the fraction with the maximum reporter ion intensity for each PSM. Next, MSstatsTMT summarized the peptides to the protein modification site level using Tukey median polish summarization (TMP). The differential abundance analysis between conditions was calculated by MSstatsTMT based on a linear mixed-effects model per protein. The inference procedure was adjusted by applying an empirical Bayes shrinkage, and resulting p-values adjusted for multiple hypothesis testing by the Benjamini-Hochberg procedure.

In the pooled TMT samples, it was observed that internal GGX peptides displayed disproportionately high signals relative to the N-terminally ubiquitinated GGX remnants. In an effort to overcome this effect and capture additional UBE2W substrates, additional immunoaffinity enrichment experiment and label-free MS analysis was performed on the Control (no dox), UBE2W only, RNF4 only, and RNF4/UBE2W (Combo) samples.

LC-MS/MS was performed similar to the two condition LFQ experiment with the following minor modifications to liquid chromatography and data acquisition. Low pH reversed phase separation was performed at a flowrate of 450 nL/minute on an Aurora Series 25 cm×75 μm I.D. column (IonOpticks). The dual-stage gradient was modified to ramp from 2 to 35 percent solvent B over 91 minutes and 35 to 75 percent over 5 minutes. For all injections, MS2 spectra were analyzed in the ion trap.

These MS data were searched using Mascot (Matrix Science) against a target-decoy database (downloaded August, 2017) containing Uniprot human and common contaminant sequences using a ppm precursor ion mass tolerance of 25 ppm, fragment ion tolerance of 0.8 Da, and semi-tryptic enzyme specificity. Carbamidomethylated cysteine (+57.0215 Da) was set as a fixed modification and methionine oxidation (+15.9949 Da), K-s-GG (+114.0429), and N-terminal GG (+114.0429) considered as variable modifications. Peptide spectral matches were filtered at the peptide level using linear discriminate analysis at a false discovery rate of 3 percent. Label-free quantitation of the N-terminal GG peptides across all data files was performed using XQuant, an algorithm guided by direct PSMs that utilizes accurate precursor ion masses and retention times to quantify peptides across runs (Kirkpatrick, D. S. et al. Proc National Acad Sci 110, 19426-19431 (2013)). Quantification and statistical testing of the label-free proteomics data was performed using MSstats v3.20.0, an open-source R/Bioconductor package (Choi, M. et al., Bioinformatics 30, 2524-2526 (2014)). Prior to MSstats analysis, PSMs were removed from further analysis if they were (1) from decoy proteins; (2) from peptides with length less than 7; (3) possessed VistaQuant confidence scores less than 71; or (4) with peak area less than 256. Redundant PSMs (i.e., multiple PSMs in one MS run that map to the same peptide) were summarized by taking the maximum intensities per run. Next, MSstats summarized the peptides to the protein modification site level using Tukey median polish summarization (TMP). The differential abundance analysis between conditions was calculated by MSstats based on a linear mixed-effects model per protein. P-values from the linear mixed-effects model were adjusted for multiple hypothesis testing by using the Benjamini-Hochberg procedure.

### Cell Culture

HEK293 cell lines were obtained from Genentech's cell line core facility gCell. Cells were maintained in Dulbecco's modified Eagle's medium (DMEM) supplemented with 10% FBS, 2 mM L-glutamine and 50 U/ml penicillin-streptomycin. All cell lines were cultured in a humidified incubator at 37° C./5% CO₂ and media was changed every other day. Where applicable, cells were treated with vehicle DMSO (Cat #D2650, Sigma-Aldrich), and 1 μg/ml doxycycline (Cat #D9891, Sigma-Aldrich), for the indicated times.

### DNA Constructs, Transfection, and Western Blotting

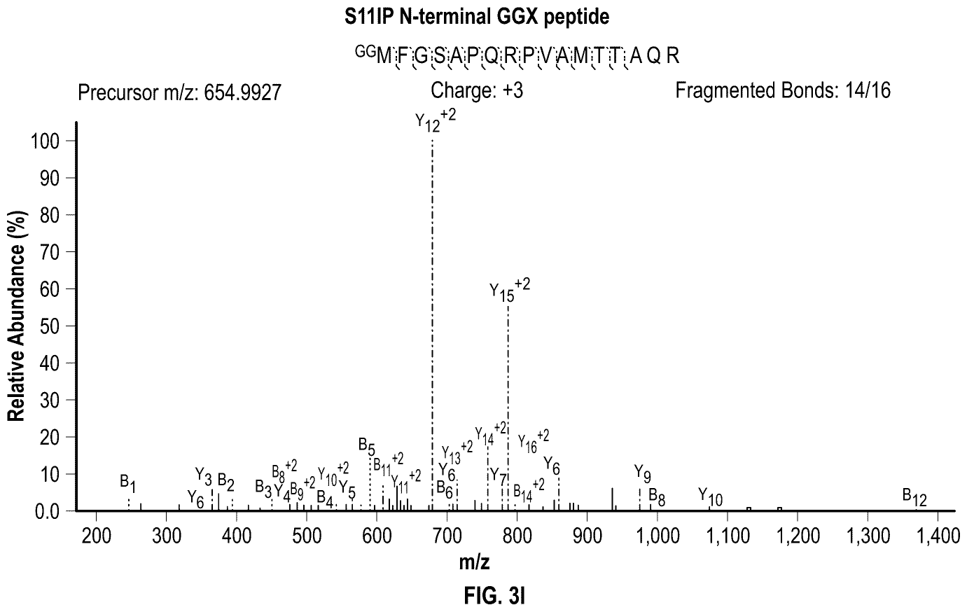

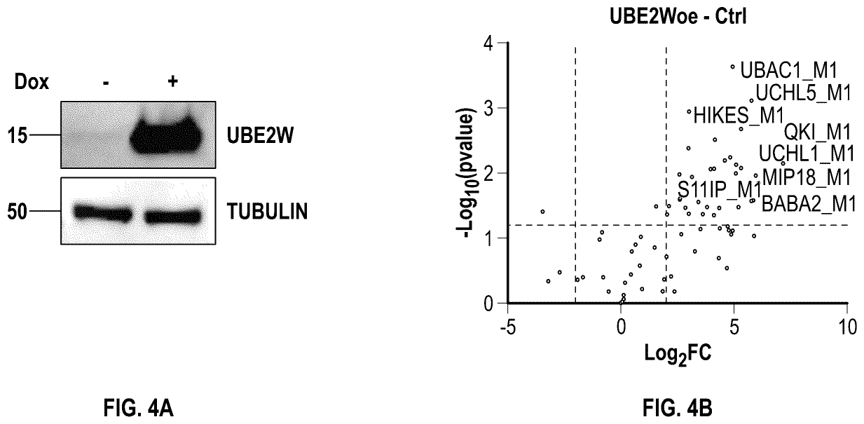

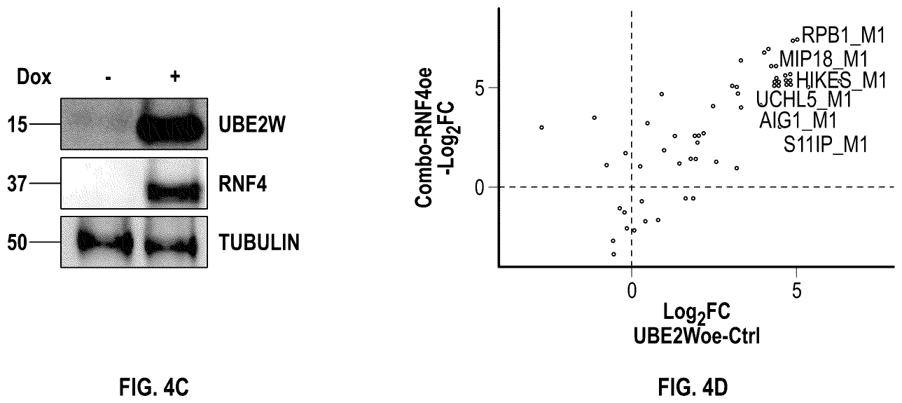

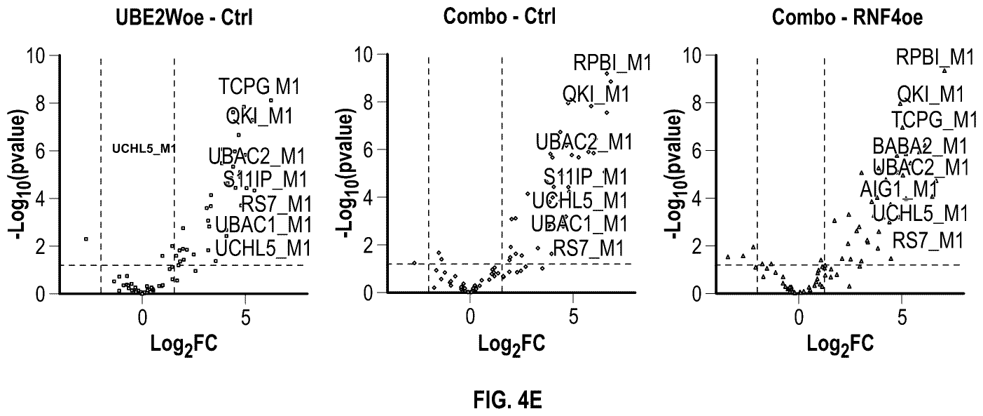

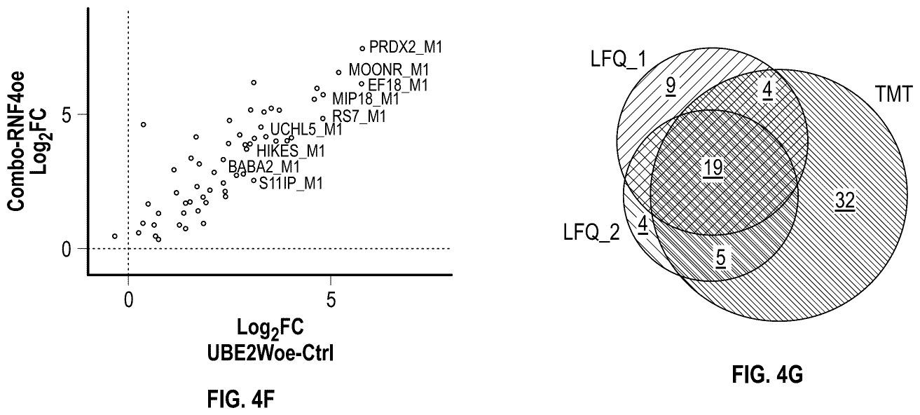

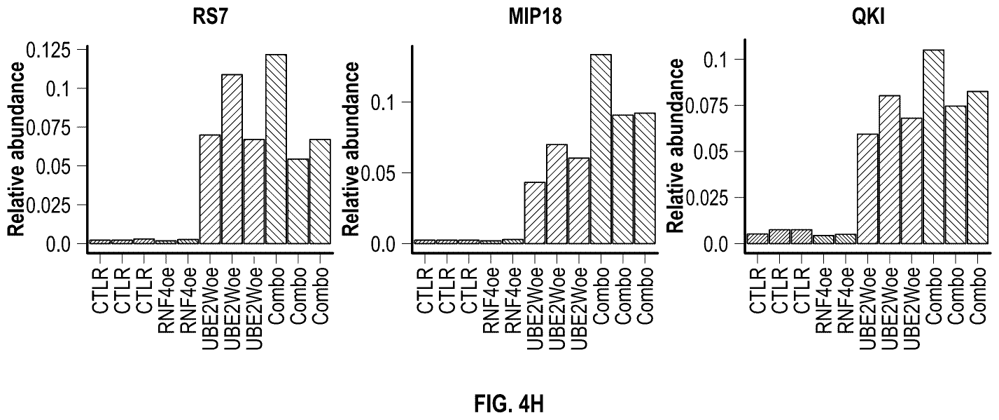

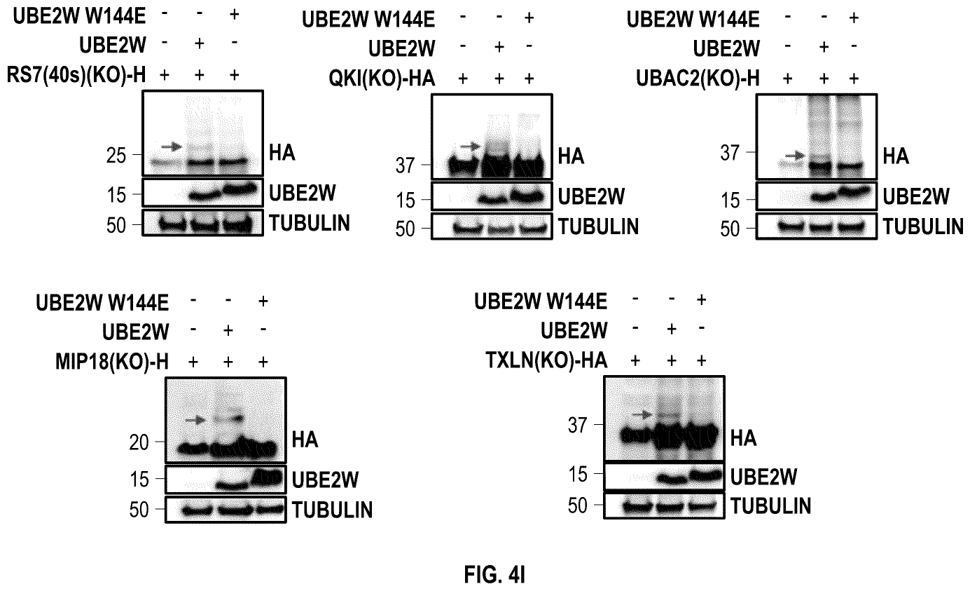

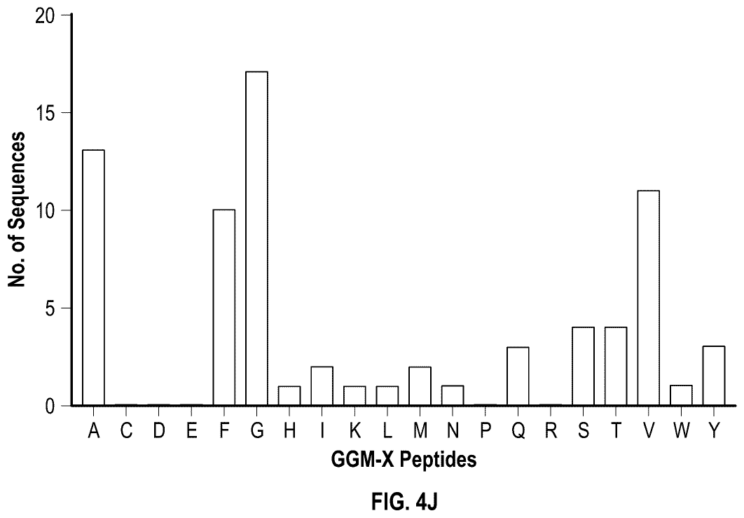

All DNA constructs were obtained by custom gene synthesis (GeneScript) and subcloned into the doxycycline-inducible piggyBac transposon plasmids (BH1.2, Genentech) using the NcoI and XhoI sites. For transient expression, HEK293 cells were seeded in 6-well plates and grown to ˜50% confluence in DMEM media. The cells were then transfected with 1 μg of piggyBac transposon plasmid by using 10 μl Fugene (promega) according to the manufacturer's instructions. For stable cell line generation, cells were cotransfected with 250 ng of piggyBac transposase plasmid (pBO, Transposagen) and 750 ng of the piggyBac transposon plasmid, using 10 μl of Fugene (promega). Three days after transfection, cells were split into selection media containing 1 μg/mL puromycin, and selected for 10 days. Stable or transient transfected cells were then assayed for protein expression by western blot analysis (see, e.g., FIG. 4A, FIG. 4C, FIG. 4I). Two days after treatment with 1 μg/mL Dox, cells were lysed with denaturing lysis buffer (9M Urea, RIPA buffer), sonicated, and centrifuged at 13,000 rpm for 10 minutes at 4° C. 15-50 μg of protein was prepared in 1×SDS loading buffer (ThermoFisher) and 1× reducing agent (ThermoFisher), heated to 90° C. for 5 min, and ran in 12% Tris-glycine gels (Bio-Rad). Gels were transferred to a nitrocellulose membrane using a Trans-Blot Turbo System (Bio-Rad) for 7 minutes at 23 V. Membranes were blocked with 5% nonfat milk diluted in PBS with 0.1% Tween® 20 (PBS-T) for 30 minutes and were rinsed briefly three times with PBS-T and incubated overnight at 4 C with primary antibodies in PBS-T with 5% BSA. Blots were washed three times for 5 minutes in PBS-T and then incubated for 1 hour at room temperature with secondary antibodies in PBS-T with 5% BSA. Blots were washed as previously described, and detection was performed with Supersignal Femto (Pierce). The antibodies were 1:5000 rabbit anti-beta Tubulin (Cat #ab6046; Abcam), 1:1000 rabbit anti-UBE2W (Cat #PA5-67547; Thermo Fisher), 1:2,000 rabbit anti-UCHL1 (Cat #HPA005993; Thermo Fisher), 1:500 mouse anti-Ubiquitin (Cat #VU-1; LifeSensors), and 1:10,000 goat anti-mouse and rabbit IgG HRP (Cat #31460 and 31430, Thermo Fisher).

### Results

Since ubiquitin-conjugating enzyme E2 (UBE2W) is the only E2 Ub-conjugating enzyme known to mediate N-terminal ubiquitination and since UBE2W expression levels are low in HEK293 cells, without wishing to be bound by theory, it was reasoned that exogenous expression of UBE2W might stimulate N-terminal ubiquitination of endogenous substrates. Therefore, a doxycycline (Dox)-inducible UBE2W HEK293 cell line was generated and used to perform a similar immunoaffinity enrichment and MS workflow as in the pilot MS experiment, as described in Example 3.

Label-free quantification (LFQ) of MS1 peak intensities was used to compare DOX+UBE2W expression to control DOX-conditions with the aim of identifying UBE2W substrates as those proteins from which GGX peptides at their N-termini increased in abundance upon UBE2W expression (FIG. 4A). Applying a similar approach as described in Example 3, peptide spectral matches (PSMs) were filtered for protein N-terminal sequences corresponding to diglycine attachment at either the initiator methionine or neo-N-terminus. In total, 152 unique GGX PSMs derived from 109 proteins were identified at peptide and protein false discovery rates of 0.80% and 3.67%, respectively. Using a criterion of log₂-fold change (log₂ FC) >1 and p<0.05 for PSMs, this experiment identified 33 UBE2W substrates (FIG. 4B, Table 8).

Most E2 Ub-conjugating enzymes work cooperatively with E3 ligases (e.g., RNF4), and previous work reported that UBE2W exhibited RNF4-dependent ubiquitination of some substrates (Tatham, M. H. et al., Biochem J 453, 137-145 (2013)). Therefore, next Dox-inducible RNF4 and bicistronic (RNF4/UBE2W, “combo”) expression vectors were generated and used to prepare stable HEK293 cell lines (FIG. 4C). An anti-GGX mAb immunoaffinity enrichment was performed, this time in concert with isobaric multiplexing via tandem mass tagging (TMT) as has been described for anti-K-ε-GG mAbs (Rose, C. M. et al., Cell Syst 3, 395-403.e4 (2016)), in addition to the LFQ approach taken above. In the TMT analysis, it was possible to compare several replicates of each condition against each other in a single multiplexed experiment: Control (no dox), UBE2W only, RNF4 only, and RNF4/UBE2W (Combo). This set of samples made it possible to evaluate potential E2/E3 synergy between UBE2W and RNF4 in the N-terminal ubiquitination of substrates. In this paradigm, UBE2W substrates are represented in two of the contrasts: UBE2W-Control and Combo-RNF4. A contrast refers to a pair of conditions being compared across the list of the identified and quantified features. In the TMT analysis, 141 unique N-terminal ubiquitinated GGX PSMs derived from 99 proteins were identified at peptide and protein false discovery rates of 0.80% and 2.02%, respectively. A cursory examination of the data revealed that RNF4 overexpression did not significantly affect N-terminal ubiquitination levels either in the RNF4 only samples or synergistically when co-expressed with UBE2W (i.e. in the Combo samples). Each of these conditions yielded similar quantitative data as the control and UBE2W only conditions, respectively. To look for hits emerging from multiple conditions, the log₂ FC of the Combo-RNF4 contrast was compared to that of UBE2W-Control, yielding a high confidence set of 60 UBE2W substrates with log₂ FC>1 and p<0.05 across multiple conditions. (FIG. 4D, Table 8).

In the corresponding LFQ analysis, 186 unique N-terminally ubiquitinated GGX PSMs derived from 120 proteins were identified at a peptide false discovery rate of 1.38%. The protein false discovery rate of this dataset was abnormally high at 13.33%, owing to the frequency of repeat identifications at the peptide level. Focusing on proteins whose N-terminal ubiquitination levels increased (log₂ FC>1 and p<0.05) in UBE2W only versus Control, Combo versus RNF4 only, and Combo vs Control, this data yielded 38 UBE2W substrates that partially overlap with the TMT analysis (FIG. 4E). Filtering for the highest confidence hits by requiring log₂ FC>1 and p<0.05 in both UBE2W-Control and Combo-RNF4 only contrasts yielded 28 high confidence UBE2W substrate protein hits (FIG. 4F).

Integrating all of the MS experimental data described herein revealed significant overlap in the identified substrates, with a subset of unique substrates identified in each of the individual experiments (FIG. 4G). Further inspection revealed that the majority (˜53%) were shared between the UBE2W alone and the UBE2W/RNF4 Combo conditions, confirming that exogenous expression of RNF4 did not enhance the activity of UBE2W in vivo (FIG. 4E, Table 8).

Collectively from three quantitative immunoaffinity enrichment experiments: LFQ interrogating UBE2W overexpression versus control, TMT analysis comparing UBE2W and RNF4 overexpression individually and in combination versus Control, and the follow up LFQ experiment, 74 UBE2W substrates are reported as reaching statistical significance (see summary in Table 8, below). The quantitative data arising from the TMT experiment consistently produced an increased signal for several proteins from samples expressing UBE2W compared to the controls, thus indicating that these are indeed substrates of UBE2W (see results for RS7, MIP18, and QKI in FIG. 4H).

Next, the newly identified substrates were validated by ectopically expressing lysine-less C-terminally HA-tagged proteins with UBE2W or UBE2W^{W144E}, a mutant that reduces ubiquitin binding, in cells (Vittal, V. et al., Nat Chem Biol 11, 83-89 (2015)). Using C-terminally tagged substrates is critical given the previous observation that an N-terminal HA-tag represents an intrinsically disordered sequences that can be recognized and modified by UBE2W (Vittal, V. et al., Nat Chem Biol 11, 83-89 (2015)). Most of the identified UBE2W substrates showed enrichment of the monoubiquitinated form; however, in some instances, high molecular weight bands could be detected consistent with polyubiquitination that, without wishing to be bound by theory, is posited to occur subsequent to N-terminal ubiquitination through the actions of other enzymes (FIG. 4I). Importantly, monoubiquitinated species of these substrates did not accumulate in cells expressing the mutant UBE2W^{W144E}, confirming that these substrates depend upon UBE2W ubiquitin binding and subsequent transfer to the target protein amino terminus (FIG. 4I). Inspection of the identified substrates across experiments revealed that N-terminal ubiquitination occurred exclusively on the translation-initiating methionine. This was surprising because many of these same proteins display small, hydrophobic amino acids in the second position which tends to trigger removal of the N-terminal Met by MetAP39 (see FIG. 4J, in which an analysis of the second position of immunoaffinity enriched UBE2W substrates indicates a preferential enrichment for peptides that contain glycine, alanine, valine, or phenylalanine after the initiator methionine). Since UBE2W is known to preferentially ubiquitinate proteins with disordered N-termini (Vittal, V. et al., Nat Chem Biol 11, 83-89 (2015)), protein prediction software was used to assess if these putative substrates indeed had disordered N-termini. Using protein DisOrder prediction System (PrDOS) (Ishida, T. & Kinoshita, K. Nucleic Acids Res 35, W460-W464 (2007)), 62 of these proteins were found to have predicted disordered N-termini (Table 8, right column).

Taken together, 74 cellular substrates of UBE2W were identified. A summary of the UBE2W substrates, as well as the proteins with putative N-terminal ubiquitination sites as identified in the pilot immunoaffinity enrichment and MS experiment described in Example 3, is provided in Table 8, below. In Table 8, an “X” indicates that the protein was identified in the corresponding MS experiment, or was predicted to have a disordered N-termini.

TABLE 8
Summary of proteins identified in immunoaffinity enrichment and MS experiments

| | | Identified in MS Experiment? | | | | Disordered |
|---|---|---|---|---|---|---|
| UniProt Entry | UniProt | Pilot (see | | | | N-termini |
| Name | Accession No. | Example 3) | LFQ_1 | LFQ_2 | TMT | predicted? |
| AAAT_HUMAN | Q15758 | | | X | | X |
| AES_HUMAN | Q08117 | | X | | | X |
| AIG1_HUMAN | Q9NVV5 | | X | X | X | X |
| ARF1_HUMAN | P84077 | | | X | X | X |
| ARL5B_HUMAN | Q96KC2 | | | X | X | — |
| BABA2_HUMAN | Q9NXR7 | | X | X | X | X |
| BUB3_HUMAN | O43684 | | X | | X | X |
| C1TC_HUMAN | P11586 | | | | X | X |
| C2AIL_HUMAN | Q96HQ2 | | X | X | X | X |
| C9J470_HUMAN | C9J470 | | | | X | X |
| CD81_HUMAN | P60033 | | X | | X | — |
| CDC45_HUMAN | O75419 | | X | X | X | X |
| DCTP1_HUMAN | Q9H773 | X | | | | X |
| DHRSX_HUMAN | Q8N5I4 | | | | X | X |
| DMKN_HUMAN | Q6E0U4-8 | | X | | X | X |
| E2AK1_HUMAN | Q9BQI3 | | X | | | X |
| EF1B_HUMAN | P24534 | | | | X | X |
| F13A_HUMAN | P00488 | X | | | | X |
| FA60A_HUMAN | Q9NP50 | | X | | | X |
| FBRL_HUMAN | P22087 | | X | | | X |
| FLOT1_HUMAN | O75955 | | | | X | — |
| GCYB1_HUMAN | Q02153 | | X | | | — |
| GOT1B_HUMAN | Q9Y3E0 | | | | X | X |
| GPAA1_HUMAN | O43292 | | X | X | X | X |
| HIKES_HUMAN | Q53FT3 | | X | X | X | — |
| HNRPK_HUMAN | P61978 | X | | | | X |
| IMPA3_HUMAN | Q9NX62 | | | | X | X |
| LAT3_HUMAN | O75387 | | | | X | X |
| LAT4_HUMAN | Q8N370 | | | | X | X |
| LRWD1_HUMAN | Q9UFC0 | | | | X | X |
| MED25_HUMAN | Q71SY5 | | X | | X | X |
| MFS12_HUMAN | Q6NUT3 | | X | X | X | X |
| MIP18_HUMAN | Q9Y3D0 | | X | X | X | X |
| MMGT1_HUMAN | Q8N4V1 | | X | X | X | X |
| MOONR_HUMAN | Q2KHM9 | | | | X | X |
| NARR_HUMAN | PODI83 | | | | X | X |
| NDUB6_HUMAN | O95139 | | | | X | X |
| NENF_HUMAN | Q9UMX5 | | | | X | X |
| NOL6_HUMAN | Q9H6R4 | | | X | X | X |
| NOP10_HUMAN | Q9NPE3 | | | | X | — |
| NUDC_HUMAN | Q9Y266 | | | | X | X |
| P121A_HUMAN | Q96HA1 | | | | X | X |
| PIGC_HUMAN | Q92535 | | X | X | X | X |
| PLBL2_HUMAN | Q8NHP8 | | | | X | X |
| PRDX1_HUMAN | Q06830 | | | | X | X |
| PRDX2_HUMAN | P32119 | | | | X | X |
| PUR9_HUMAN | P31939 | X | X | X | X | X |
| QKI_HUMAN | Q96PU8 | | X | X | X | X |
| RAD21_HUMAN | O60216 | | | | X | X |
| RCAS1_HUMAN | O00559 | | | | X | X |
| REEP1_HUMAN | Q9H902 | | X | | | — |
| RFA1_HUMAN | P27694 | X | X | X | X | X |
| RPB1_HUMAN | P24928 | | X | X | X | X |
| RPB7_HUMAN | P62487 | X | | | | — |
| RS29_HUMAN | P62273 | | | | X | X |
| RS7_HUMAN | P62081 | | X | X | X | X |
| S11IP_HUMAN | Q8N1F8 | X | X | X | X | X |
| SGMR1_HUMAN | Q99720 | | X | X | X | X |
| T179B_HUMAN | Q7Z7N9 | | | | X | X |
| TAF1_HUMAN | P21675 | | | | X | X |
| TCPG_HUMAN | P49368 | | | X | X | X |
| TF3C4_HUMAN | Q9UKN8 | | | | X | X |
| TM127_HUMAN | O75204 | | | | X | X |
| TMM97_HUMAN | Q5BJF2 | | | | X | X |
| TMX2_HUMAN | Q9Y320 | | | X | | X |
| TSN13_HUMAN | O95857 | | X | X | X | — |
| TSN3_HUMAN | O60637 | | | X | | — |
| TTC27_HUMAN | Q6P3X3 | | X | | | X |
| UBAC1_HUMAN | Q9BSL1 | | X | | | X |
| UBAC2_HUMAN | Q8NBM4 | | | X | | X |
| UCHL1_HUMAN | P09936 | | X | | | — |
| UCHL5_HUMAN | Q9Y5K5 | X | X | X | X | X |
| VKOR1_HUMAN | Q9BQB6 | | | | X | X |
| VRK3_HUMAN | Q8IV63 | | | | X | — |
| ZDH12_HUMAN | Q96GR4 | | | | X | X |
| ZN253_HUMAN | O75346 | | | X | X | — |
| ZN672_HUMAN | Q499Z4 | | | | X | X |

### Example 5: UCHL1 and UCHL5 are Substrates of UBE2W, and N-Terminal Ubiquitination Regulates UCHL1 and UCHL5 Deubiquitinase Activity

The following example describes experiments characterizing the UBE2W substrates UCHL1 and UCHL5, two members of the ubiquitin C-terminal hydrolase (UCH) family of deubiquitinases. Specifically, it was demonstrated that UCHL1 and UCHL5 are N-terminally ubiquitinated by UBE2W in in vitro ubiquitination assays. Further, N-terminal ubiquitination was shown to modulate the deubiquitinase activity of UCHL1 and UCHL5.

### Materials and Methods

### Cell Culture

HEK293 and COS-7 cell lines were obtained from Genentech's cell line core facility gCell. Cells were maintained in Dulbecco's modified Eagle's medium (DMEM) supplemented with 10% FBS, 2 mM L-glutamine and 50 U/ml penicillin-streptomycin. All cell lines were cultured in a humidified incubator at 37° C./5% CO₂ and media was changed every other day. Where applicable, cells were treated with vehicle DMSO (Cat #D2650, Sigma-Aldrich), 1 μg/ml doxycycline (Cat #D9891, Sigma-Aldrich), 1 μM Bortezomib (Cat #2204, CST), and 10 μg/ml cycloheximide (Cat #2112, CST) for the indicated times.

### Ubiquitination Assays

For ubiquitination assays a mix of 100 nM E1 (Cat #E-305, Boston Biochem), 4 M UBE2W (Cat #E2-740, Boston Biochem), 1 M UCHL1-KO and UCHL5-KO (made in house), 1 μM RNF4 (Cat #E3-210, Boston Biochem), and 250 μM Ub (Cat #U-100H, Boston Biochem) was used. All reactions were performed in 40 μl ubiquitination buffer (50 mM tris pH 7.5, 5 mM MgCl₂, 50 mM KCL, and 0.2 mM DTT) at 37° C. for 2 hours. Reactions were started with 3 mM ATP and stopped by addition of Laemmli buffer and heated to 90° C., followed by separation of proteins by SDS-PAGE and visualization by immunoblotting with the appropriate antibodies.

### Biolayer Interferometry Assay

BLI assays were run on Octet Red384 (Forte Bio) platform using 384 tilted-well plates. All experiments were conducted at 25° C., 1000 RPM shaking, 60 μL well volume, and used buffer containing: 150 mM NaCl, 20 mM Tris 7.5, 1 mg/mL BSA, 0.01% Tween®-20, and 1 mM TCEP. 60 second baseline or wash steps preceded each loading, association, or dissociation step in blank buffer. Immobilization of biotinylated ubiquitin (Cat #UB-570, Boston Biochem) was optimized on Streptavidin biosensors (Cat #18-5019, Forte Bio) to 18 nM (0.156 μg/L) for a 1 nM response over a 300 second loading step. Association of the wildtype or ubiquitin-fused proteins was carried out a 4-fold dilution of protein starting at 5

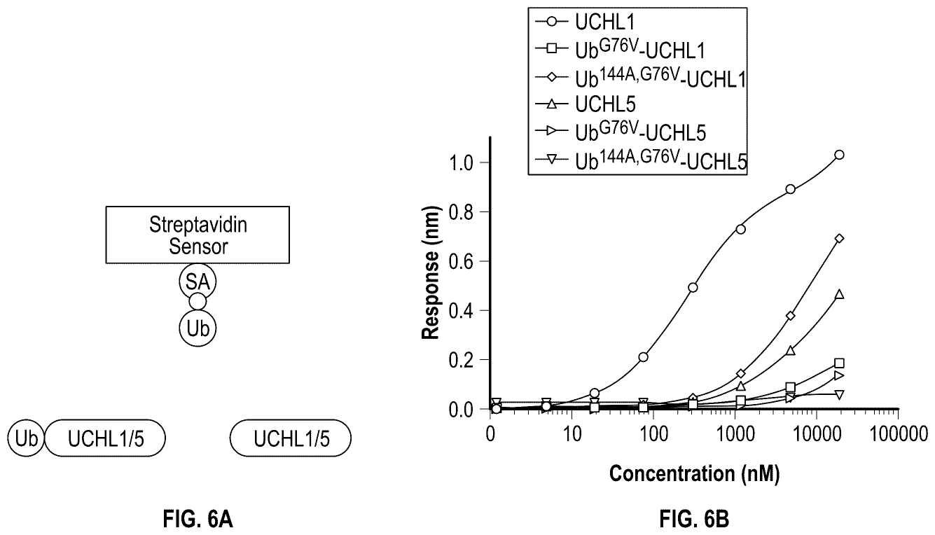

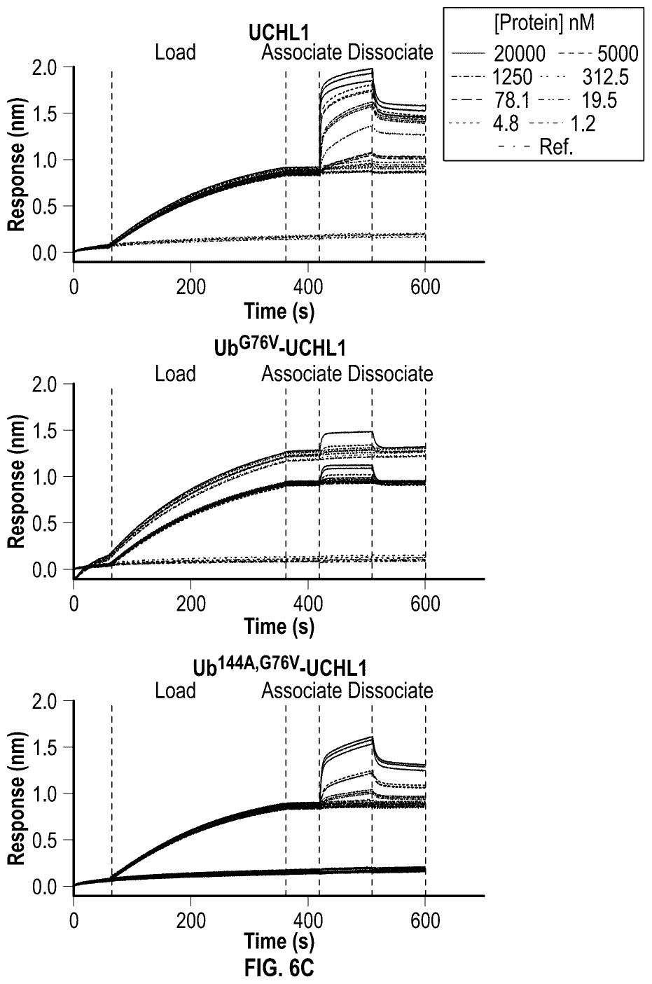

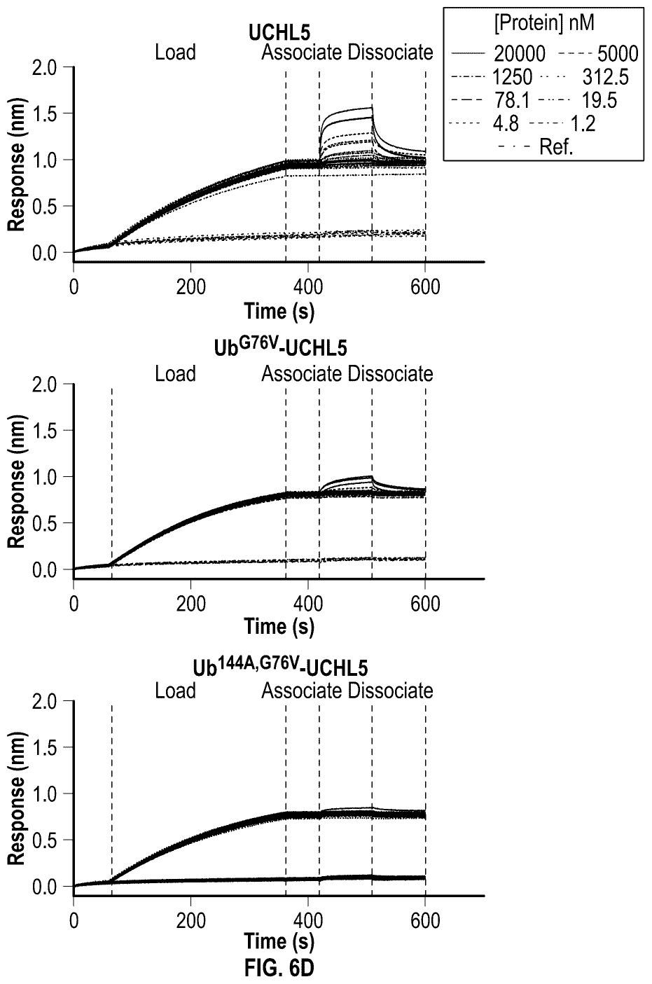

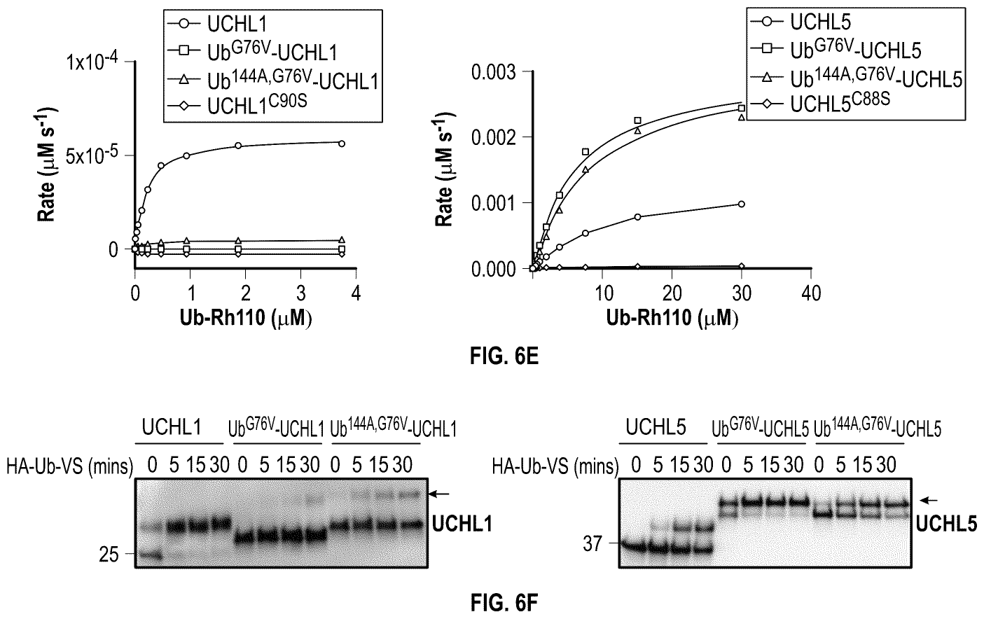

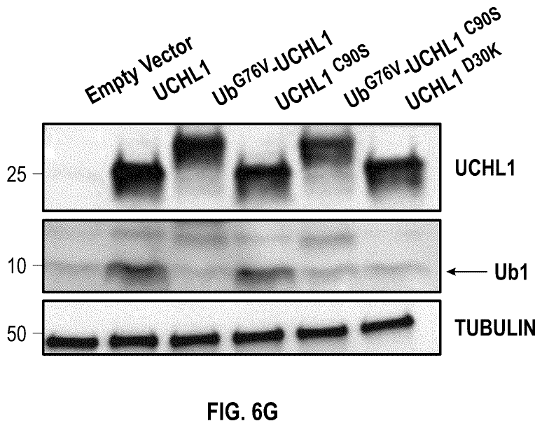

M. The association step was measured for 180 seconds, and the dissociation step was measured for 300 seconds. Streptavidin tips with no biotin-ubiquitin loaded were used to measure the non-specific binding of each protein dilution with the tip surface, and the data was subtracted from the raw data measurements before curve fitting. Association and dissociation curves were fitted using a 1:1 binding model in prism, with K_{D} being determined from kinetic constants as well as steady state measurements (see FIG. 6A, FIG. 6B, FIG. 6C, and FIG. 6D).

### Ub-Rhodamine 110 Enzymatic Assays

Ub-Rhodamine 110 (Cat #U-555, Boston Biochem) was dissolved in DMSO, and activity assays were determined using 1 nM of purified enzyme with 0.5 μM of substrate (Ub-Rho110) in 10 μl reaction buffer (50 mM HEPES pH 7.5, 50 mM KCl, 5% glycerol, 5 mM MgCl₂, 5 mM DTT, 0.1 mg/ml BSA, and 0.005% Tween-20). Experiments were performed at 37° C. in black 384-well non-binding surface low flange plates (Corning) and monitored in a EnVision® 2105 Multimode Plate Reader (PerkinElmer) using 350 nm and 450 nm excitation and emission wavelengths respectively. Measurements were taken every 60 seconds for 120 minutes (see FIG. 6E).

### Ubiquitin Vinyl Sulfone Assays

Purified proteins (50 nM) were subjected to enzymatic reactions with 1 μM Ub vinyl sulfone HA-tagged probe (Ub-VS-HA) (Cat #U-212, Boston Biochem). All reactions were done in 40 μl of deubiquitinase (DUB) buffer (50 mM HEPES pH 7.5, 50 mM KCl, 5% glycerol, 5 mM MgCl₂, 5 mM DTT, 0.1 mg/ml BSA, and 0.005% Tween®-20) at 37° C. for 30 minutes. Modification of enzymes by site-directed HA-Ub-VS probes were detected by immunoblotting with the appropriate antibodies (see FIG. 6F).

### Protein Expression and Purification

All full-length wild type and mutant proteins, as well as N-terminally fused Ub, were obtained by custom gene synthesis (GeneScript) and subcloned into single protein expression vectors for expression in E. coli. All sequences were tagged at their C-termini with 6-His tags. Proteins were expressed in BL21-Gold(DE3) cells for 18 hours at 18° C., then harvested by centrifugation in lysis buffer containing 500 mM NaCl, 50 mM Tris 7.5, 5% Glycerol, and 1 mM TCEP. Proteins were purified by affinity chromatography (Ni-NTA Agarose, Thermofisher) followed by size exclusion chromatography (16/600 Superdex200, GE Healthcare). Protein samples were concentrated and frozen down in GF Buffer (150 mM NaCl, 20 mM Tris 7.5, 1 mM TCEP).

### Results

### UCHL1 and UCHL5 are Substrates of UBE2W

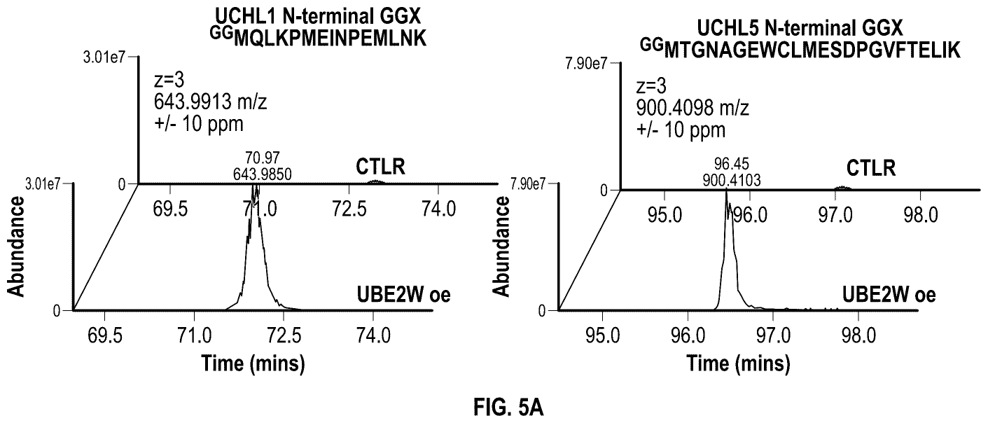

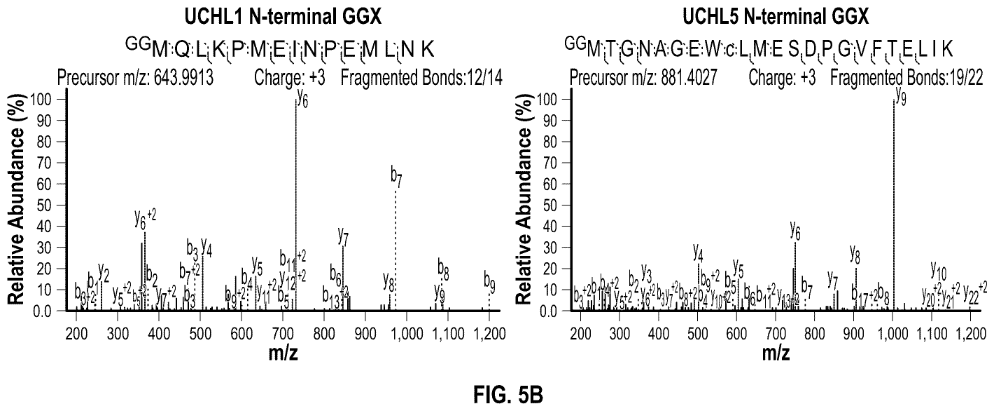

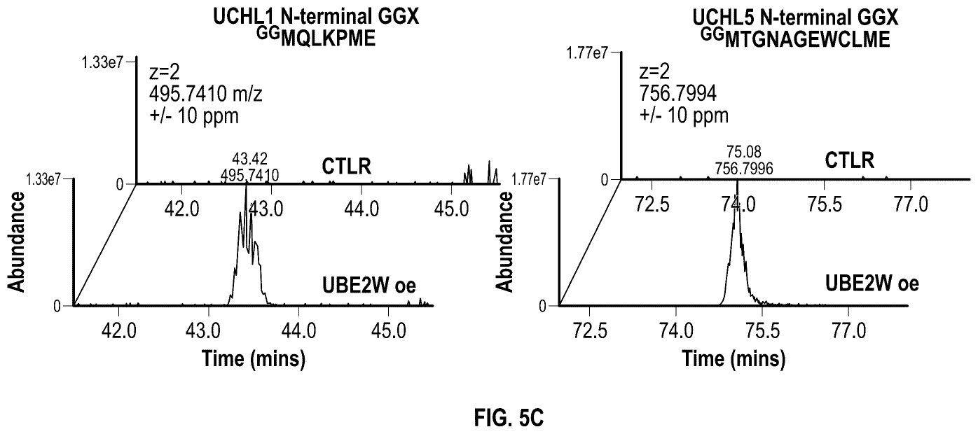

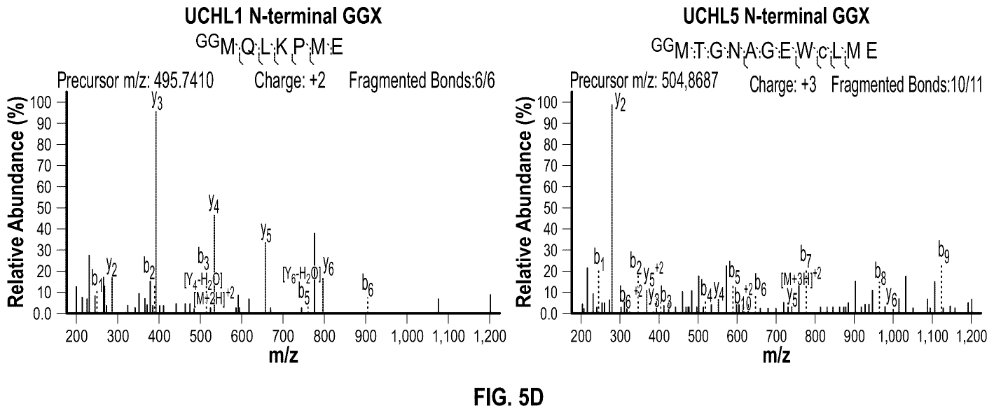

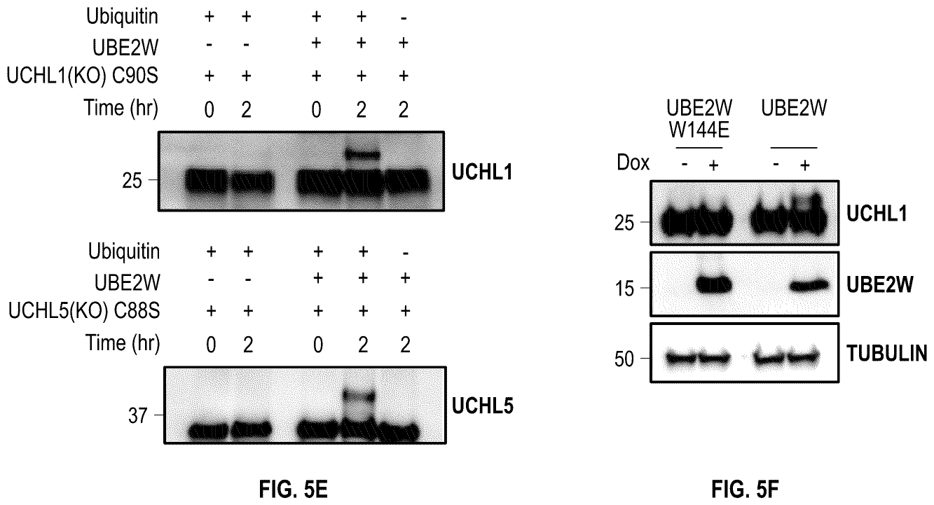

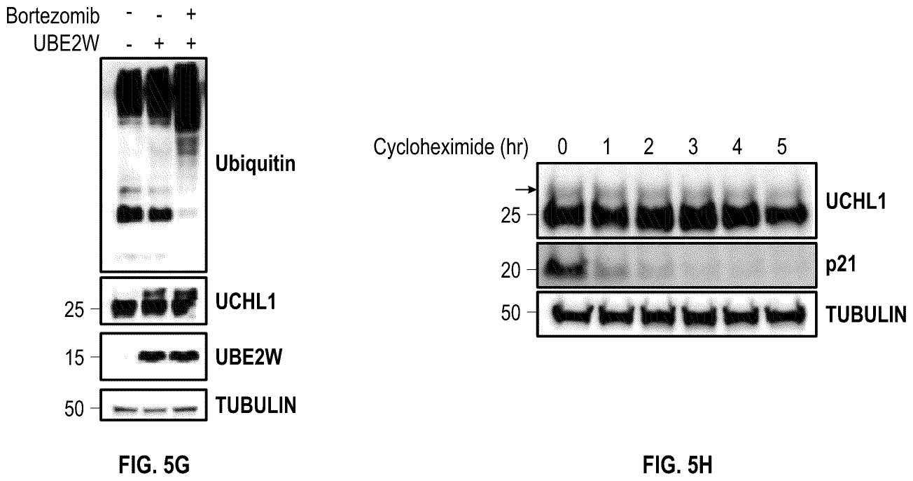

Notable amongst the UBE2W substrate list were two members of the Ubiquitin C-terminal hydrolase (UCH) family of deubiquitinases, UCHL1 and UCHL5 (FIG. 5A, FIG. 5B, FIG. 5C, FIG. 5D, Table 8). UCHL1 was identified as a putative substrate in one of three LC-MS experiments, while UCHL5 was identified in all three. Due to the idiosyncratic nature of data dependent shotgun sequencing, these experiments did not produce data to demonstrate N-terminal ubiquitination of UCHL1 in TMT sample. Two distinct forms of N-terminally ubiquitinated UCHL1 and UCHL5 peptides were identified, representing the semi-tryptic and fully tryptic forms of each, further increasing confidence in their respective identifications. Previously, it had been suggested that UCHL1 is N-terminally ubiquitinated, however the enzyme responsible for this modification was unknown (Meray, R. K. & Lansbury, P. T. J Biol Chem 282, 10567-10575 (2007)). To validate that these two deubiquitinases were indeed N-terminally ubiquitinated, in vitro ubiquitination assays were performed with purified proteins, and it was observed that UBE2W can monoubiquitinate lysine-less versions of both UCHL1 and UCHL5 (FIG. 5E). Supporting these data, endogenous UCHL1 was monoubiquitinated upon expression of UBE2W in cells (FIG. 5F). Importantly, UBE2W^{W144E} expression did not support the formation of Ub-UCHL1 (FIG. 5F). Unfortunately, the modification of UCHL5 was not detected in western blot experiments (data not shown).

N-terminal ubiquitination has been proposed to be a signal for protein degradation (Ciechanover, A. & Ben-Saadon, R. Trends Cell Biol 14, 103-106 (2004); Breitschopf, K. et al., Embo J 17, 5964-5973 (1998); Bloom, J. et al., Cell 115, 71-82 (2003); Coulombe, P. et al., Mol Cell Biol 24, 6140-6150 (2004)). However, it was recently shown that N-terminally ubiquitinated proteins accumulate only modestly in the presence of proteasome inhibitors, suggesting that N-terminal ubiquitination might have roles aside from protein degradation (Akimov, V. et al., Nat Struct Mol Biol 25, 631-640 (2018)). Therefore, whether N-terminal ubiquitination promotes the degradation of UCHL1 in cellular assays was evaluated. To test this, UBE2W was expressed and HEK293 cells were treated with the proteasome inhibitor Bortezomib. Although high molecular weight ubiquitinated proteins accumulated in cells treated with Bortezomib, an accumulation of Ub-UCHL1 was not observed (FIG. 5G). To reconfirm that the N-terminal monoubiquitination of UCHL1 does not trigger its degradation, cycloheximide chase experiments were performed. While the labile protein p21 was quickly degraded in cells expressing UBE2W and treated with cycloheximide, Ub-UCHL1 remained stable. Only at a later time point (5 hours) did UCHL1 protein levels start to decline (FIG. 5H). Altogether, these results suggested that N-terminal monoubiquitination by UBE2W in cells did not trigger degradation of UCHL1.

### N-Terminal Ubiquitination Regulates UCHL1 and UCHL5 Deubiquitinase Activity

Since N-terminal ubiquitination did not promote UCHL1 degradation in the cell-based assays, whether N-terminal ubiquitination modulated UCHL1 and UCHL5 deubiquitinase function was next evaluated. To assess this, a number of UCHL1 and UCHL5 variants were generated, including wild-type (UCHL1^{WT} and UCHL5^{WT}), catalytically inactive mutants (UCHL1^{C90S} and UCHL5^{C88S}), N-terminally ubiquitinated mimetics (Ub^{G76V}_UCHL1 and Ub^{G76V}_UCHL5), and a ubiquitin mutant (Ub^{I44A,G76V}_UCHL1 and Ub^{I44A,G76V}_UCHL5). For the mimetics, the C-terminus of ubiquitin was fused to the initial methionine of the deubiquitinase, and the last glycine in ubiquitin was mutated to valine to prevent removal of ubiquitin via UCH autocatalytic activity.

Previous structural work showed that Ub binding causes UCHL1 active site residues to rearrange into a catalytically competent configuration (Boudreaux, D. A. et al., Proc National Acad Sci 107, 9117-9122 (2010)). However, monoubiquitination of internal lysines near the active site of UCHL1 has been shown to block the binding of its substrates (Meray, R. K. & Lansbury, P. T. J Biol Chem 282, 10567-10575 (2007)). Moreover, the activity of UCHL5 is tuned at the level of substrate affinity (Yao, T. et al., Nat Cell Biol 8, 994-1002 (2006)). Therefore, whether N-terminal ubiquitination would modulate the ubiquitin binding affinities of UCHL1 and UCHL5 was tested. Using Bio-Layer interferometry (BLI), the monoubiquitin binding capabilities of UCHL1^{WT}, UCHL5^{WT}, Ub^{G76V}_UCHL1, Ub^{G76V}_UCHL5, Ub^{I44A,G76V}_UCHL1, and Ub^{I44A,G76V}-UCHL5 was examined (FIG. 6A). Consistent with previous reports (Larsen, C. N. et al., Biochemistry-us 37, 3358-3368 (1998); Osaka, H. et al., Hum Mol Genet 12, 1945-1958 (2003)), a strong interaction was observed between monoubiquitin and UCHL1^{WT}. However, binding was only observed between monoubiquitin and Ub^{G76V}_UCHL1 at high monoubiquitin concentrations (5 μM) (FIG. 6B, FIG. 6C). Similar trends were seen for UCHL5, however, the affinity for monoubiquitin was much reduced compared to UCHL1 (FIG. 6B, FIG. 6D). Interestingly, Ub^{I44A,G76V}_UCHL1 showed a ˜3-fold increase in binding compared to Ub^{G76V}_UCHL1, suggesting that UCHL1 is able to interact in cis with its N-terminal Ub modification. In contrast, the addition of I44A had no impact on Ub binding for Ub^{G76V}_UCHL1. Thus, it was concluded that N-terminal ubiquitination prevents UCHL1 and UCHL5 from binding monoubiquitin.

Next, whether the deubiquitinase activity of UCHL1 and UCHL5 was altered upon N-terminal ubiquitination was examined by performing deubiquitinase activity assays using Ubiquitin-Rhodamine 110 (Ub-Rho110). The kinetics of UCHL1^{WT} and UCHL5^{WT} agreed with previous reports (Boudreaux, D. A. et al., Proc National Acad Sci 107, 9117-9122 (2010); Yao, T. et al., Nat Cell Biol 8, 994-1002 (2006)), and, as expected, no activity was detectable from catalytically dead UCHL1^{C90S} and UCHL5^{C88S} (FIG. 6E and Table 9). Strikingly, N-terminal ubiquitination of UCHL1 and UCHL5 conferred opposite effects on their respective deubiquitinase activities. Ub^{G76V}_UCHL1 and Ub^{I44A,G76V}_UCHL1 showed significantly reduced activity compared to UCHL1^{WT}, whereas Ub^{G76V}_UCHL5 and Ub^{I44A,G76V}_UCHL5 had a significantly enhanced activity compared to UCHL5^{WT} (FIG. 6E and Table 9). To corroborate the Ub-Rho110 assays, the suicide probe Ubiquitin-Vinyl Sulfone (Ub-VS) was used (Borodovsky, A. et al., Embo J 20, 5187-5196 (2001)). UCHL1^{WT} readily reacted with Ub-VS, nearing completion after 30 minutes, whereas Ub^{G76V}_UCHL1 remained largely unmodified (FIG. 6F). Conversely, UCHL5^{WT} was only partially modified at 30 minutes, whereas Ub^{G76V}_UCHL5 rapidly reacted with Ub-VS (FIG. 6F). Altogether, these data demonstrate that N-terminal ubiquitination modulated the deubiquitinase activities of both UCHL1 and UCHL5, but in opposite directions.

TABLE 9
UCHL1 and UCHL5 kinetics table for
Ub Rho-110-monoubiquitin experiment

| Protein | Kₘ (μM) | k_{cat} (s⁻¹) | k_{cat}/Kₘ (s⁻¹ μM⁻¹) |
|---|---|---|---|
| UCHL1 | 0.2009 | 0.093 | 0.4 |
| Ub^{G67V}-UCHL1 | 0.13 | 0.000267 | 0.002 |
| Ub^{I44A, G76V}-UCHL1 | 0.198 | 0.005356 | 0.027 |
| UCHL1^{C90S} | ND | ND | ND |
| UCHL5 | 12.38 | 2.792 | 0.22 |
| Ub^{G67V}-UCHL5 | 6.395 | 6.112 | 0.95 |
| Ub^{I44A, G76V}-UCHL5 | 8.875 | 6.268 | 0.70 |
| UCHL5^{C88S} | ND | ND | ND |

(“ND” indicates no data)

Finally, previous reports have indicated that UCHL1 deubiquitinase activity regulates the free monoubiquitin pool in cells (Osaka, H. et al., Hum Mol Genet 12, 1945-1958 (2003)). Since N-terminal ubiquitination negatively regulated UCHL1 activity in vitro, the physiological consequences of this modification in cells was explored. Consistent with previous work in COS-7 cells (Meray, R. K. & Lansbury, P. T. J Biol Chem 282, 10567-10575 (2007)), exogenous expression of UCHL1^{WT} significantly increased the levels of free monoubiquitin (FIG. 6G, lane 2 compared to lane 1). However, expression of Ub^{G76V}-UCHL1 reduced the accumulation of free monoubiquitin to background levels (FIG. 6G, lane 3). This result supports the in vitro biochemical observations showing that Ub^{G76V}-UCHL1 is unable to bind to monoubiquitin (FIG. 6B, FIG. 6C). UCHL1^{C90S} expression triggered accumulation of free monoubiquitin similarly to UCHL1^{WT} (FIG. 6G, compare lane 4 to lane 2). However, cells expressing Ub^{G76V}_UCHL1^{C90S} and a non-Ub binding UCHL1 mutant (UCHL1^{D30K}) showed basal levels of free monoubiquitin (FIG. 6G, lanes 5 and 6).

Collectively, the data presented herein indicated that the Ub-binding activity and not the catalytic activity of UCHL1 regulated the pool of free monoubiquitin in cells. Moreover, these results demonstrated that N-terminal ubiquitination of UCHL1 blocked Ub binding and inhibited its cellular function.

### Conclusion

In summary, the work described herein establishes a new antibody toolkit for globally profiling N-terminally ubiquitinated polypeptides and identifies a new role for this non-canonical form of ubiquitination. The key enzyme responsible for synthesizing this post-translational modification has been characterized, and insight has been provided into and how substrates are modified at their N-termini.

### Example 6: Demonstration that N-Terminal Ubiquitination Functions Independently of Proteasomal Degradation

The following example describes experiments testing whether N-terminal ubiquitination results in proteasomal degradation of a large set of substrates. Although the data demonstrated that UCHL1 was not targeted for proteasomal degradation upon N-terminal ubiquitination, the existing data did not rule out a broader link between N-terminal ubiquitination and proteasomal degradation. To systematically evaluate this connection, the UBE2W Dox-inducible overexpression model was used, this time in the presence and absence of proteasome inhibitor Bortezomib (Btz).

### Materials and Methods

Experiments were performed as described in Example 4 using label free quantification. Cells were additionally treated with the proteasome inhibitor Bortezomib (Btz) at 10 μM for 2 hours before cell harvest.

### Results

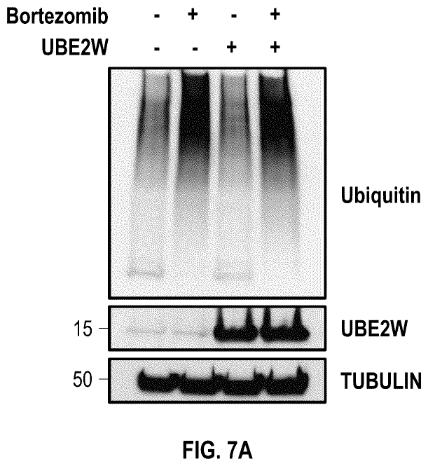

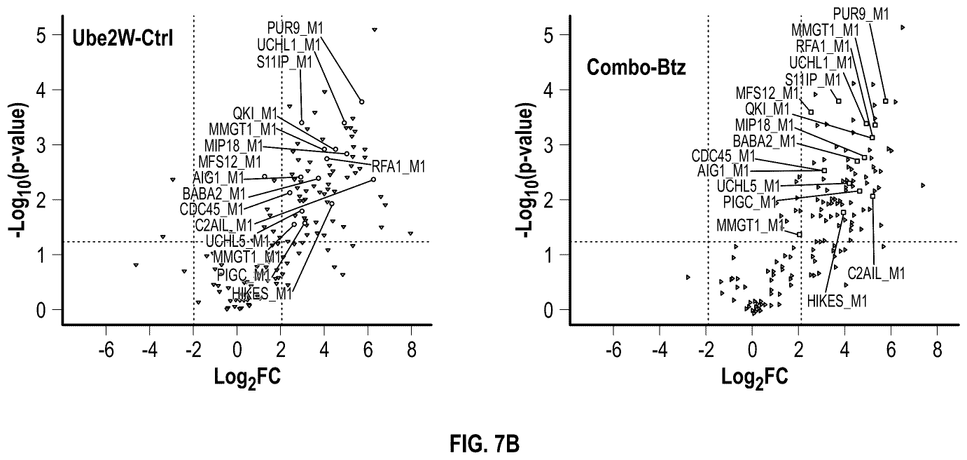

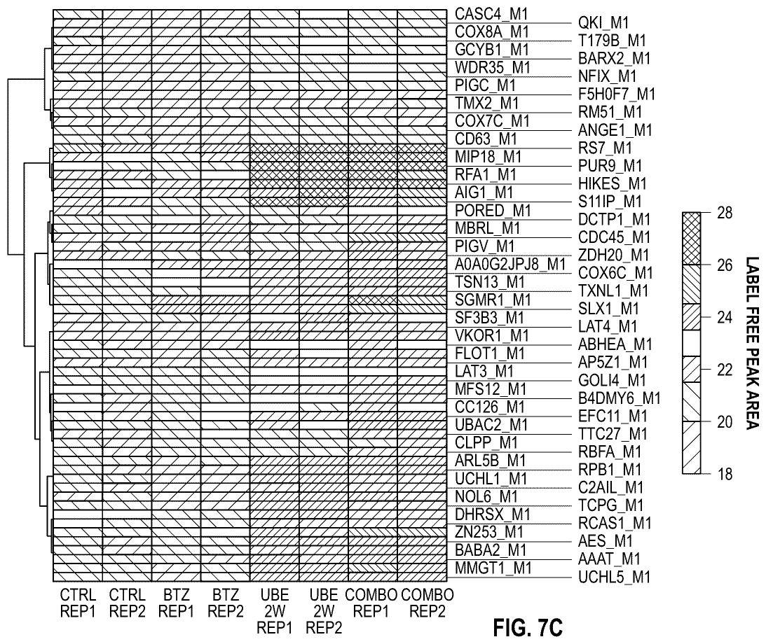

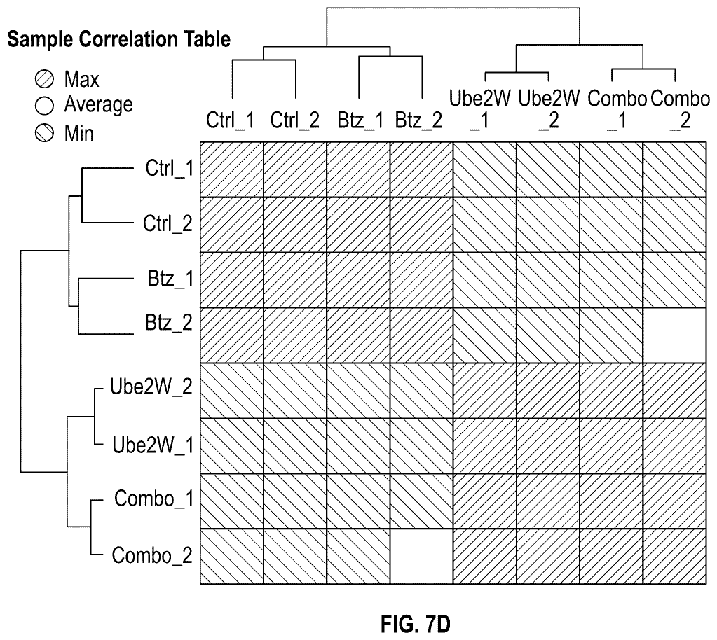

Four individual samples were generated in biological duplicates: the Control (Dox (−)/Btz (−)), Btz proteasome inhibition alone (Dox (−)/Btz (+)), UBE2W overexpression alone (Dox (+)/Btz (−)), and Combo (Dox (+)/Btz (+)) (FIG. 7A). Using the same filtering and cutoffs as for the previous MS experiments, label free quantitative data for GGX enriched peptides were interrogated to ask whether N-terminal ubiquitination increased upon proteasome inhibition. As in the earlier RNF4 experiments, UBE2W-dependent substrates were identified both in the absence and presence Btz treatment, as represented by the UBE2W-Ctrl (left, FIG. 7B) and Combo-Btz (right, FIG. 7B) contrasts. Interestingly, GGX enriched peptide abundances were systematically unaltered by proteasome inhibition across a wide range of substrates (FIG. 7C), confirming the hypothesis that proteasomal degradation was not a major consequence of N-terminal ubiquitination. Consistent with this observation, there was a strong correlation between UBE2W overexpressing conditions (i.e. UBE2W and Combo), whereas Btz-treated samples closely mirrored the controls (FIG. 7D). However, 12 out of the 236 GGX peptides (˜5%) showed a coordinate increase in abundance in the Combo sample relative to either the Btz treatment alone or UBE2W overexpression, as represented by a Log₂FC>2 in both the Combo-UBE2W and Combo-Btz contrasts. Taken together, the label-free proteomic analysis described herein confirmed that N-terminal ubiquitination by UBE2W was not sufficient to trigger proteasomal degradation on the vast majority of substrates.

### Example 7: Use of Anti-GGX Antibodies to Detect Ubiquitinated Polypeptides

The following example describes the digestion of cell lysates with the protease Lb^{pro}*, and the use of anti-GGX antibodies to detect peptides with a Gly-Gly motif in the digested cell lysate. Lb^{pro}* cleaves peptide bonds preceding Gly-Gly amino acid residues. Accordingly, compared to trypsin, which primarily cleaves peptide chains at the carboxyl side of lysine or arginine amino acid residues, Lb^{pro}* selectively cleaves proteins with a greater degree of sequence specificity. Because proteins lacking a Gly-Gly motif will not be cleaved by Lb^{pro}*, it is believed that the use of Lb^{pro}* to digest a cell lysate will result in a pool of digested peptides or modified proteins enriched for peptides from ubiquitinated substrates.

### Preparation of Lb^{pro}*, and Ub-clipping

Lb^{pro}* is expressed and purified according to the protocol described in Swatek, K. N. et al., Protocol Exchange 2019 Aug. 22; 10.21203/rs.2.10850/v1. Further, “Ub-clipping” is performed using whole cell lysates (ibid.). Specifically, cell lysates are incubated with Lb^{pro} to produce modified proteins containing a GG addition at sites of ubiquitination.

### Detection of Ubiquitinated Polypeptides.

Ubiquitinated polypeptides in the cell lysate are then detected by western blot using an anti-GGX antibody provided herein. Specifically, whole cell lysates that have been digested with Lb^{pro}* are loaded and separated on an SDS-PAGE gel, and transferred to a membrane using techniques standard in the art. Alternatively, whole cell lysates that have been digested with Lbpro* can be subjected to an immunoprecipitation with an anti-GGX antibody prior to loading onto the SDS-PAGE gel. The membrane is incubated with one or more anti-GGX antibodies. The GGX antibody can be labeled or detected using a secondary antibody, for example an anti-rabbit antibody.

What is claimed is:

1. An antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG).

2. The antibody of claim 1, wherein the antibody binds to a peptide comprising an N-terminal sequence selected from the group consisting of GGA, GGE, GGF, GGG, GGH, GGI, GGL, GGM, GGN, GGQ, GGS, GGT, GGV, and GGW.

3. The antibody of claim 1 or claim 2, wherein the antibody binds to a peptide comprising the N-terminal sequence of GGA, a peptide comprising the N-terminal sequence of GGE, a peptide comprising the N-terminal sequence of GGF, a peptide comprising the N-terminal sequence of GGG, a peptide comprising the N-terminal sequence of GGH, a peptide comprising the N-terminal sequence of GGI, a peptide comprising the N-terminal sequence of GGL, a peptide comprising the N-terminal sequence of GGM, a peptide comprising the N-terminal sequence of GGN, a peptide comprising the N-terminal sequence of GGQ, a peptide comprising the N-terminal sequence of GGS, a peptide comprising the N-terminal sequence of GGT, a peptide comprising the N-terminal sequence of GGV, and a peptide comprising the N-terminal sequence of GGW.

4. The antibody of any one of claims 1-3, wherein the antibody is a rabbit, rodent, or goat antibody.

5. The antibody of any one of claims 1-4, wherein the antibody is a full-length antibody or a Fab fragment.

6. The antibody of any one of claims 1-5, wherein the antibody is conjugated to a detectable label.

7. The antibody of claim 6, wherein the label is selected from the group consisting of biotin, digoxigenin, and fluorescein.

8. The antibody of any one of claims 1-7, wherein the antibody is immobilized on a solid support.

9. The antibody of claim 8, wherein the antibody is immobilized on a bead.

10. The antibody of any one of claims 1-9, wherein the antibody comprises a variable heavy chain (VH) comprising an Asn at position 35, Val at position 37, Thr at position 93, Asn at position 101, and Trp at position 103 on one side, and a variable light chain (VL) comprising an Ala at position 34, a Tyr at position 36, and a Tyr at position 49, numbering according to Kabat.

11. The antibody of any one of claims 1-9, wherein the antibody comprises a variable heavy chain (VH) and a variable light chain (VL), wherein the antibody comprise a CDRH1 comprising the amino acid sequence XXXMN (SEQ ID NO: 35); a CDRH2 comprising the amino acid sequence XXXXXGXXYYATWA (SEQ ID NO:36); and a CDRH3 comprising the amino acid sequence DDXXXXNX (SEQ ID NO:37); wherein the antibody comprises a CDRL1 comprising the amino acid sequence QSXXSVYXXNXLX (SEQ ID NO:38); a CDRL2 comprising the amino acid sequence XASTLXS (SEQ ID NO: 39); and a CDRL3 comprising the amino acid sequence LGXXDCXSXDCXX (SEQ ID NO:40); wherein X is any amino acid.

12. The antibody of claim 11, wherein the VH comprises the amino acid set forth in SEQ ID NO: 33 and the VL comprises the amino acid sequence set forth in SEQ ID NO:34.

13. The antibody of any one of claims 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 1 and a CDRL1, CDRL2, and CDRL3 of a VL comprising the amino acid sequence set forth in SEQ ID NO: 2.

14. The antibody of claim 13, wherein the antibody comprises the CDRH1 amino acid sequence set forth in SEQ ID NO: 3; the CDRH2 amino acid sequence set forth in SEQ ID NO: 4; the CDRH3 amino acid sequence set forth in SEQ ID NO:5; the CDRL1 amino acid sequence set forth in SEQ ID NO: 6; the CDRL2 amino acid sequence set forth in SEQ ID NO:7; and the CDRL3 amino acid sequence set forth in SEQ ID NO:8.

15. The antibody of claim 13, wherein the VH comprise the amino acid sequence set forth in SEQ ID NO: 1 and the VL comprises the amino acid sequence set forth in SEQ ID NO:2.

16. The antibody of any one of claims 13-15, wherein the antibody comprises a heavy chain and a light chain, wherein the heavy chain comprises the amino acid sequence set forth in SEQ ID NO: 52, and the light chain comprises the amino acid set forth in SEQ ID NO: 53.

17. The antibody of any one of claims 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 9 and a CDRL1, CDRL2, and CDRL3 of a VL comprising the amino acid sequences set forth in SEQ ID NO: 10.

18. The antibody of claim 17, wherein the antibody comprises the CDRH1 amino acid sequence set forth in SEQ ID NO: 11; the CDRH2 amino acid sequence set forth in SEQ ID NO: 12; the CDRH3 amino acid sequence set forth in SEQ ID NO:13; the CDRL1 amino acid sequence set forth in SEQ ID NO: 14; the CDRL2 amino acid sequence set forth in SEQ ID NO:15; and the CDRL3 amino acid sequence set forth in SEQ ID NO:16.

19. The antibody of claim 18, wherein the VH comprises the amino acid sequence set forth in SEQ ID NO: 9 the VL comprises the amino acid sequence set forth in SEQ ID NO:10.

20. The antibody of any one of claims 17-19, wherein the antibody comprises a heavy chain and a light chain, wherein the heavy chain comprises the amino acid sequence set forth in SEQ ID NO: 54, and the light chain comprises the amino acid set forth in SEQ ID NO: 55.

21. The antibody of any one of claims 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 17 and a CDRL1, CDRL2, and CDRL3 of a VL comprising the amino acid sequence set forth in SEQ ID NO: 18.

22. The antibody of claim 21, wherein the antibody comprises the CDRH1 amino acid sequence set forth in SEQ ID NO: 19; the CDRH2 amino acid sequence set forth in SEQ ID NO: 20; the CDRH3 amino acid sequence set forth in SEQ ID NO:21; the CDRL1 amino acid sequence set forth in SEQ ID NO: 22; the CDRL2 amino acid sequence set forth in SEQ ID NO:23; and the CDRL3 amino acid sequence set forth in SEQ ID NO:24.

23. The antibody of claim 22, wherein the VH comprises the amino acid set forth in SEQ ID NO: 17 and the VL comprises the amino acid set forth in SEQ ID NO:18.

24. The antibody of any one of claims 21-23, wherein the antibody comprises a heavy chain and a light chain, wherein the heavy chain comprises the amino acid sequence set forth in SEQ ID NO: 56, and the light chain comprises the amino acid set forth in SEQ ID NO: 57.

25. The antibody of any one of claims 1-9, wherein the antibody comprises a variable heavy chain (VH) and a variable light chain (VL), wherein the antibody comprises a CDRH1, a CDRH2, and a CDRH3 of a VH comprising the amino acid sequence set forth in SEQ ID NO: 25 and a CDRL1, CDRL2, and CDRL3 of a VL comprising the amino acid sequences set forth in SEQ ID NO: 26.

26. The antibody of claim 25, wherein the antibody comprises the CDRH1 amino acid sequence set forth in SEQ ID NO: 27; the CDRH2 amino acid sequence set forth in SEQ ID NO: 28; the CDRH3 amino acid sequence set forth in SEQ ID NO:29; a CDRL1 amino acid sequence set forth in SEQ ID NO: 30; the CDRL2 amino acid sequence set forth in SEQ ID NO:31; and the CDRL3 amino acid sequence set forth in SEQ ID NO:32.

27. The antibody of claim 26, wherein the VH comprises the amino acid sequence set forth in SEQ ID NO: 25 and the VL comprises the amino acid sequence set forth in SEQ ID NO: 26.

28. The antibody of any one of claims 25-27, wherein the antibody comprises a heavy chain and a light chain, wherein the heavy chain comprises the amino acid sequence set forth in SEQ ID NO: 58, and the light chain comprises the amino acid set forth in SEQ ID NO: 59.

29. Nucleic acid encoding the antibody of any one of claims 1-28.

30. A host cell comprising the nucleic acid of claim 29.

31. A method of screening for an antibody that binds to a peptide of an N-terminally ubiquitinated polypeptide, wherein the antibody binds to the amino acid sequence GGX at the N-terminus of the peptide, wherein the antibody does not bind to an amino acid sequence comprising a branched diglycine (K-ε-GG), the method comprising
i) providing an antibody library;
ii) positively selecting antibodies that bind to a peptide comprising the amino acid sequence GGX at the N-terminus, wherein X is any amino acid; and
iii) negatively selecting antibodies that bind to a peptide comprising the amino acid sequence K-ε-GG, thereby producing an antibody that specifically binds to a peptide comprising the amino acid GGX at the N-terminus, and does not bind to the amino acid sequence K-ε-GG.

32. The method of claim 31, wherein in step ii) antibodies that bind to a peptide comprising the amino acid sequence GGM at the N-terminus are positively selected.

33. The method of claim 31 or 32, wherein negatively selecting antibodies that bind to a peptide comprising the amino acid sequence K-ε-GG is performed simultaneously with step ii).

34. The method of claim 31 or 32 wherein negatively selecting antibodies that bind to a peptide comprising the amino acid sequence K-ε-GG before or after step ii).

35. The method of any one of claims 31-34, wherein the library is a phage library or a yeast library.

36. The method of any one of claims 31-35, wherein the library is produced by immunizing a mammal with a peptide library comprising peptides comprising the amino acid sequence GGM at the N-terminus.

37. The method of claim 36 wherein the mammal is a rabbit or a mouse.

38. The method of any one of claims 31-37, wherein steps ii)-iii) are repeated two or more times.

39. An antibody produced by the method of any one of claims 31-38.

40. A method of enriching peptides of N-terminally ubiquitinated proteins in a sample comprising a mixture of peptides, comprising:
i) contacting the sample with an antibody that binds to a peptide of an N-terminally ubiquitinated protein; and
ii) selecting antibody-bound peptides from the sample, wherein the antibody binds to the amino acid sequence GGX at the N-terminus, wherein the antibody does not bind to an amino acid sequence comprising a branched diglycine (K-ε-GG).

41. The method of claim 40, wherein the sample is a cell lysate.

42. The method of claim 41, further comprising deleting a deubiquitinase in a cell and lysing the cell to produce the cell lysate.

43. The method of claim 41, further comprising overexpressing a ubiquitin ligase in a cell and lysing the cell to produce the cell lysate.

44. The method of any one of claims 41-43, wherein the cell lysate is incubated with trypsin to generate the peptides.

45. The method of any one of claims 41-43, wherein the cell lysate is incubated with a bacterial or viral protease to generate the peptides.

46. The method of any one of claims 42-45, further comprising treating the cell with a proteasome inhibitor or an inhibitor of de-ubiquitination prior to lysate generation and incubation with trypsin or prior to incubation with the bacterial or viral protease.

47. The method of any one of claims 40-46, further comprising detecting the selected antibody-bound peptides.

48. The method of claim 47, wherein the antibody-bound peptides are detected by mass spectrometry.

49. The method of claim 47, wherein the antibody-bound peptides are detected by protein sequencing.

50. The method of claim 47, wherein the antibody-bound peptides are detected using a secondary antibody that binds to the antibody that binds to a peptide of an N-terminally ubiquitinated protein.

51. A library of peptides of N-terminally ubiquitinated proteins produced by the method of any one of claims 40-50.

52. A method of detecting a peptide of an N-terminally ubiquitinated protein in a sample comprising a mixture of peptides comprising
i) incubating the sample with an enzyme to generate peptides;
ii) contacting the peptides with an antibody that binds to a peptide of an N-terminally ubiquitinated protein, and
iii) detecting the peptide, wherein the antibody binds to the amino acid sequence GGX at the N-terminus, wherein the antibody does not bind to an amino acid sequence comprising a branched diglycine (K-ε-GG).

53. The method of claim 52, wherein the peptide is detected using a secondary antibody that binds to the antibody that binds to a peptide of an N-terminally ubiquitinated protein.

54. The method of claim 52 or 53, wherein the sample is a cell lysate.

55. The method of claim 54, further comprising deleting a deubiquitinase in a cell and lysing the cell to produce the cell lysate.

56. The method of 54, further comprising overexpressing a ubiquitin ligase in a cell and lysing the cell to produce the cell lysate.

57. The method of any one of claims 54-56, wherein the cell lysate is incubated with a bacterial or viral protease to generate the peptides.

58. The method of any one of claims 55-57, further comprising treating the cell with a proteasome inhibitor or an inhibitor of de-ubiquitination prior to lysate generation and incubation with the bacterial or viral protease.

59. A kit for detecting peptide of an N-terminally ubiquitinated protein in a sample comprising an antibody that binds to a peptide of an N-terminally ubiquitinated polypeptide and instructions for use, wherein the antibody that binds to a peptide of an N-terminally ubiquitinated polypeptide, wherein the antibody binds to the amino acid sequence GGX at the N-terminus, wherein the antibody does not bind to an amino acid sequence comprising a branched diglycine (K-ε-GG).

60. The kit of claim 59, wherein the antibody is conjugated to a detectable label.

61. The kit of claim 60, wherein the detectable label is selected from the group consisting of biotin, digoxigenin, and fluorescein.

62. The kit of any one of claims 59-61, wherein the antibody is immobilized on a solid support.

63. The kit of claim 62, wherein the antibody is immobilized on a bead.

64. The kit of any one of claims 59-63, further comprising a protease.
